(19) United States
(12) Patent Application Publication
(10) Pub. No.: US 2024/0140983 A1
(43) Pub. Date: May 2, 2024

(54) SINGLE STRANDED DNA ENZYME INHIBITORS

(71) Applicant: MASSEY UNIVERSITY, Palmerston North (NZ)

(72) Inventors: Fareeda Maged Yahya BARZAK, Palmerston North (NZ); Vyacheslav Vladimirovich FILICHEV, Palmerston North (NZ); Elena HARJES, Palmerston North (NZ); Stefan HARJES, Palmerston North (NZ); Geoffrey Brind JAMESON, Palmerston North (NZ); Harikrishnan Mohana KURUP, Palmerston North (NZ); Maksim V. KVACH, Minsk (BY); Yongdong SU, Palmerston North (NZ)

(21) Appl. No.: 18/262,896

(22) PCT Filed: Jan. 26, 2022
(86) PCT No.: PCT/IB2022/050656

§ 371 Date: Jul. 25, 2023

(30) Foreign Application Priority Data
Jan. 26, 2021 (AU) 2021900164

(51) Int. Cl. C07H 21/04

(52) U.S. Cl. CPC C07H 21/04

(57) ABSTRACT

The invention relates to single stranded DNA inhibitors of APOBEC3 enzymes and uses thereof. The compounds of the invention comprise β-D-5-O-phosphato-2-deoxyribofuranosyl analogues of nucleoside-like inhibitors of cytidine deaminase, flanked by oligonucleotide sequences which may be cross-linked. The compounds of the invention may have therapeutic application against diseases and conditions in which inhibition of a human cytidine deaminase of the APOBEC3 family would be beneficial.

### 1. FIELD OF THE INVENTION

The invention relates to inhibitors of APOBEC3 enzymes and uses thereof.

### 2. BACKGROUND OF THE INVENTION

Nucleic acid editing by APOBEC3 (apolipoprotein B mRNA editing enzyme catalytic polypeptide-like 3; termed “A3” herein) family members is an important part of the innate immune system involved in combating pathogens but often contributing to viral evolution and having detrimental consequences in cancers. Unlike single nucleotide cytidine deaminases (CDA) which act on individual cytidines, APOBEC enzymes deaminate cytosine to uracil (C-U) (FIG. 1A) on single-stranded DNA (ssDNA) only.

A3 catalytic activity is dependent on zinc (Zn²⁺)-mediated hydrolysis of the 4-NH₂ group on cytosine residues in ssDNA. The A3 enzymes contain a consensus Zn²⁺-binding motif of histidine(His)-X-glutamic acid(Glu)-X₂₃₋₂₈-proline(Pro)-cysteine(Cys)-X₂₋₄-cysteine(Cys), where X represents any amino acid and the His and Cys residues coordinate Zn²⁺. The active site also contains a water molecule, yielding a tetrahedrally coordinated Zn²⁺.

A3 enzymes cannot turn over a single cytidine nucleoside. The substrate to be deaminated must include at least a 4-mer DNA oligomer, where the targeted cytosine residue is proceeded by two nucleotides on its 5′ end and one nucleotide on its 3′ end.

There are seven human A3 enzymes (A3A-A3H, excluding A3E). Although A3 enzymes are a part of the human immune system, they can also mutate human genomic DNA and thus contribute to cancer genesis, cancer mutagenesis, and the development of drug resistance. For example, single-domain A3A and double-domain A3B have been identified as the major contributors to cancer mutagenesis; their mutational signatures have been found in different cancers including bladder, breast, cervix, head/neck, and lung cancers. A3B is also associated with poor survival prognosis in some cancers. Since A3A and A3B are not essential to primary metabolism and A3B deletion is prevalent in some populations, their inhibition (especially of nucleus-targeted A3B) offers a potent strategy to suppress cancer evolution, thereby making existing anti-cancer therapies more efficient and longer lasting. A3 enzymes are known contributors to viral evolution (human immunodeficiency virus (HIV), Rubella and SARS-CoV-2) leading to formation of mutated viral strains. As A3 enzymes deaminate predominantly ssDNA, the inventors have previously theorized that ssDNA-based inhibitors of A3 enzymes may be useful as conjuvants to existing cancer therapies.

To date, no potent small-molecule inhibitors of A3A and A3B have been described in the literature. Current approaches to inhibiting A3 enzymes include the use of ssDNAs into which the cytidine nucleoside analogues 2′-deoxyzebularine (dZ) or 5-flouro-2′-deoxyzebularine (FdZ) have been incorporated.¹⁻² However, the low in vitro inhibitory potential of such dZ or FdZ-modified ssDNAs (with Kᵢ values in the low μM range) indicates that these compounds will not be effective in vivo and so are not likely to be clinically useful. Currently, A3 enzymes are down-regulated in cells and in an organism using RNA interference.³ However, this approach involves repetitive administration of long short-hairpin RNAs in the presence of suitable transfection reagents, which is not a desirable therapeutic option due to potential toxicity and difficulties of administration over long time.

It is therefore an object of the invention to provide an APOBEC3 inhibitor that overcomes at least some of the disadvantages set out above and/or that at least provides the public with a useful choice.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### 3. SUMMARY OF THE INVENTION

In a first aspect the present invention relates to a compound of formula I

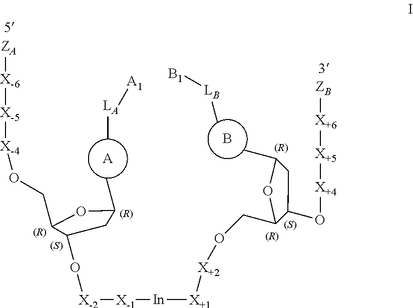

wherein
A₁ and B₁ are selected from —N₃ and —C≡CH wherein A₁ and B₁ are different;
L_{A} and L_{B} are independently absent or are independently selected from the group consisting of —(C₁-C₆)alkylene, —(C₁-C₆)alkenylene and —(C₁-C₆)alkynylene, wherein each of —(C₁-C₆)alkylene, —(C₁-C₆)alkenylene and —(C₁-C₆)alkynylene may incorporate one or two heteroatoms and/or an aryl group in the carbon chain;
the A and B rings are independently selected from a substituted or non-substituted 5-14 membered N-containing monocyclic or polycyclic heteroaryl group, wherein each A and B ring is attached to the β-D-2′-deoxyribofuranosyl unit via a N atom present in each of the A and B rings;
each of X₋₆, X₋₅, X₋₄, X₋₂, X₋₁, X₊₁, X₊₂, X₊₄, X₊₅ and X₊₆ is absent or is independently selected from a nucleotide or a nucleotide derivative;
Z_{A} and Z_{B} are each independently absent or selected from an oligonucleotide consisting of 1 to 10 nucleotides and/or nucleotide derivatives;
and In is a β-D-5-O-phosphato-2-deoxyribofuranosyl analogue of a nucleoside-like inhibitor of cytidine deaminase.

In one embodiment, In is selected from the group consisting of

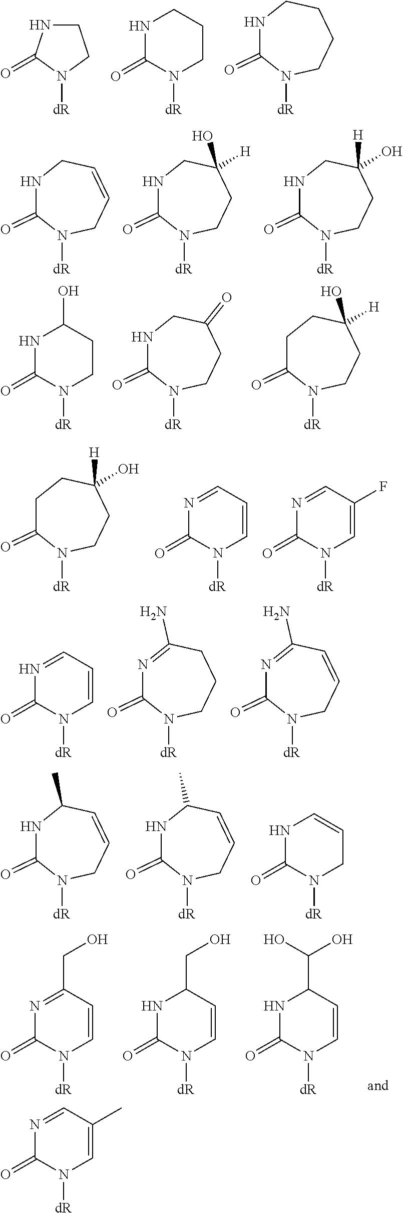

wherein dR is β-D-5-O-phosphato-2-deoxyribofuranosyl as depicted in formula I′ wherein * denotes the point of attachment to X₋₂ or X₋₁, ** denotes the point of attachment to X₊₁ or X₊₂, and *** denotes the point of attachment to the N atom of the In group

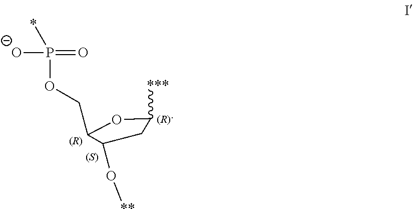

In a second aspect the invention relates to a compound of formula IIa or IIb

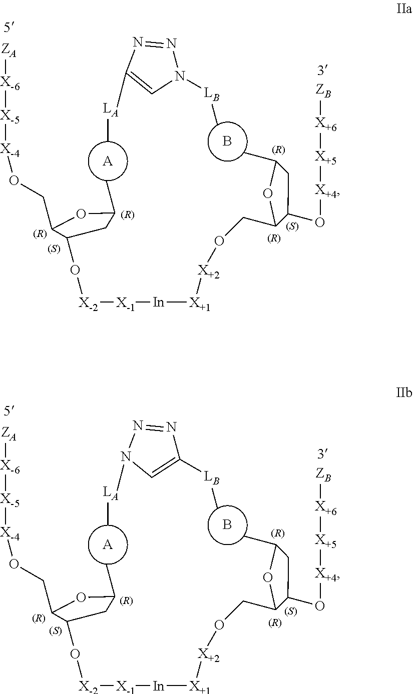

wherein L_{A}, L_{B}, A, B, X₋₆, X₋₅, X₋₄, X₋₂, X₋₁, X₊₁, X₊₂, X₊₄, X₊₅, X₊₆ and In are defined as for formula I.

In the First and Second Aspects:

In one embodiment one or both of L_{A} and L_{B} are —(C₁-C₆)alkylene or absent.

In one embodiment one or both of L_{A} and L_{B} are meta-ethynylbenzen-1-yl or 5-hexynyl-1-yl or absent.

In one embodiment each of X₋₆, X₋₅, X₋₄, X₋₂, X₋₁, X₊₁, X₊₂, X₊₄, X₊₅ and X₊₆ is a nucleotide, preferably a native nucleotide.

In one embodiment the A and B rings are independently selected from the group consisting of (a) purinyl, (b) 7-deazapurinyl, (c) 8-aza-7-deazapurinyl, (d) pyrimidinyl and (e) tricyclic or dicyclic nitrogen-containing heteroaryl.

In one embodiment the A and B rings are independently selected from the group consisting of (a) substituted purinyl, (b) substituted 7-deazapurinyl, (c) substituted 8-aza-7-deazapurinyl, (d) substituted pyrimidinyl and (e) substituted tricyclic or dicyclic nitrogen-containing heteroaryl.

In one embodiment the A and B rings are independently substituted with one or more substituents selected from the group consisting of halo, nitro, cyano, alkyl, alkoxy, amino, substituted amino, —COH, —COOH, and —CONH₂.

In one embodiment the A and B rings are independently selected from the group consisting of (a) a modified purinyl selected from the group consisting of:

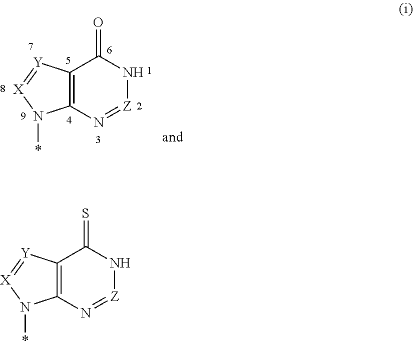

wherein X═C—R¹, Y═N and

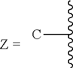

### or X═N, Y═C—R¹ and

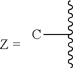

### or X═C—R¹,

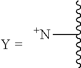 [SMILES: *[NH3+]]

### and Z═C—R² or X═N,

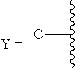

### and Z═C—R¹ or

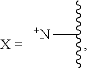 [SMILES: *[NH3+]]

Y═C—R¹ and Z═C—R² or

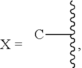

### Y═N and Z═C—R¹;

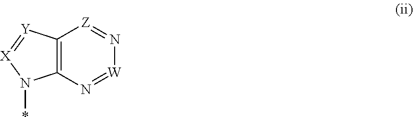

wherein X═C—R¹, Y═N,

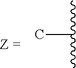

and W═C—R² or X═N, Y═C—R¹,

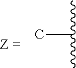

and W═C—R² or X═C—R¹,

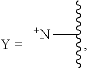 [SMILES: *[NH3+]]

Z═C—R² and W═C—R³ or X═N,

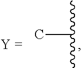

Z═C—R¹ and W═C—R² or

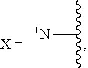 [SMILES: *[NH3+]]

Y═C—R¹, Z═C—R² and W═C—R³ or

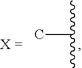

Y═N, Z═C—R¹ and W═C—R² or X═C—R¹, Y═N, Z═C—R² and

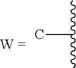

or X═N, Y═C—R¹, Z ═C—R² and

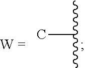

### X═C—R¹, Y═N,

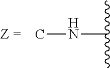 [SMILES: *NC]

and W═C—R² or X═C—R¹, Y═N, Z ═C—R² and

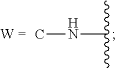 [SMILES: *NC]

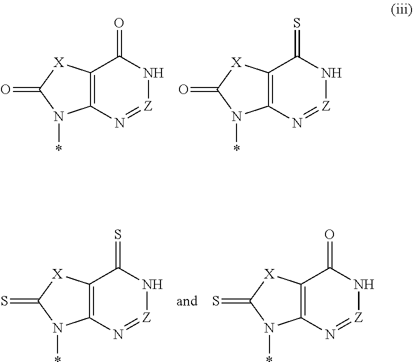

wherein X═C—R¹ and

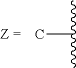

### or X═N and

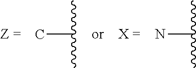

### and Z═C—R¹ or

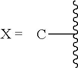

### and Z═C—R¹;

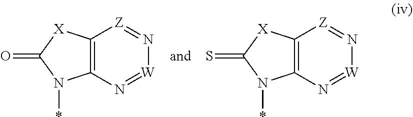

wherein X═C—R¹,

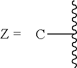

### and W═C—R² or X═N,

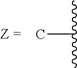

### and W═C—R¹ or

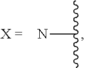 [SMILES: *N]

Z ═C—R¹ and W═C—R² or

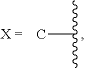

Z═C—R¹ and W═C—R² or X═C—R¹, Z═C—R² and

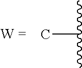

### or X═N, Z═C—R¹ and

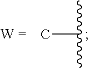

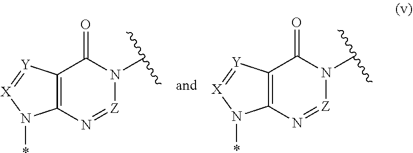

wherein X═N, Y═C—R¹ and Z═C—R² or X═C—R¹, Y═N and Z═C—R²;

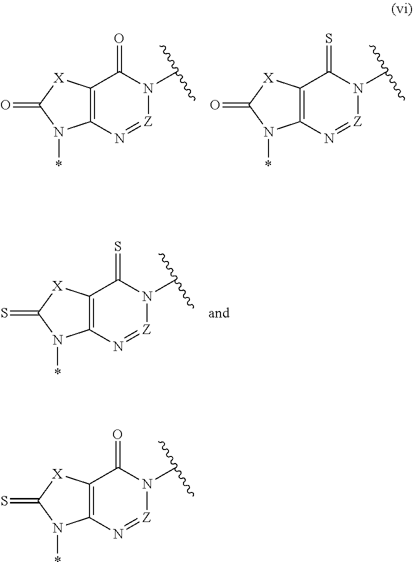

wherein X═C—R¹ and Z═C—R² or X═N and Z═C—R¹;

and wherein R¹, R² and R³ are independently selected from the group consisting of H, halo, nitro, cyano, alkyl, alkoxy, amino, mono or disubstituted amino, —COH, —COOH, and —CONH₂.

(b) a modified pyrimidine selected from the group consisting of:

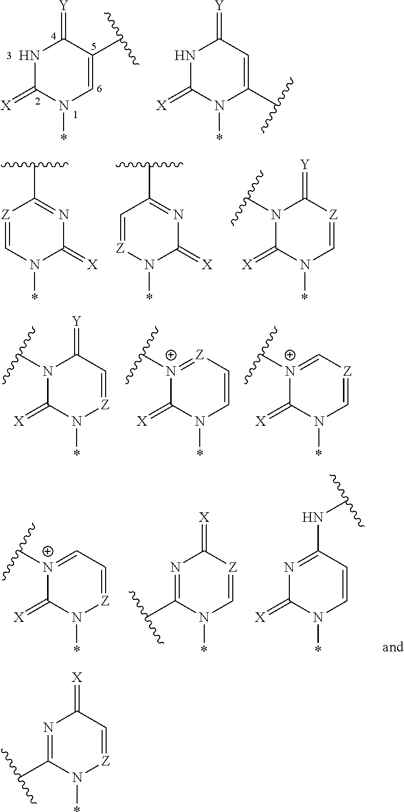

wherein X and Y are independently selected from a or S and Z is independently selected from N or C—R¹ wherein R is independently selected from the group consisting of H, halo, nitro, cyano, alkyl, alkoxy, amino, mono or disubstituted amino, —COH, —COOH, and —CONH₂;

(c) a substituted tricyclic or dicyclic or monocyclic nitrogen-containing heteroaryl selected from the group consisting of:

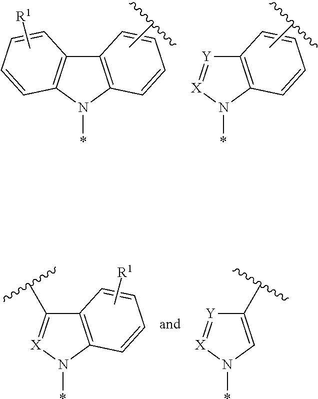

wherein X and Y are independently selected from N, O, S or C—R¹ wherein R¹ is independently selected from the group consisting of H, halo, nitro, cyano, alkyl, alkoxy, amino, mono or disubstituted amino, —COH, —COOH, and —CONH₂;

wherein for each of (a), (b) and (c)

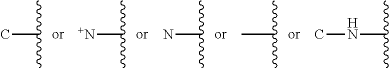

indicates the point of attachment of the ring to L_{A} or L_{B} and * indicates the point of attachment to the β-D-2′-deoxyribofuranosyl unit I′ of formula I or II.

In one embodiment A and B are independently selected from the group consisting of

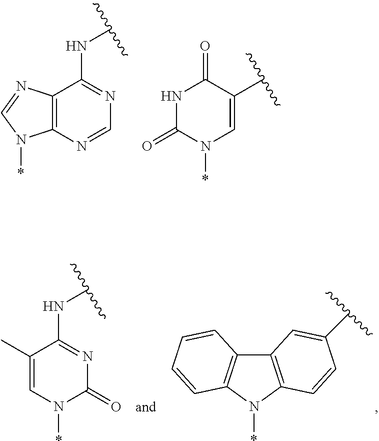

wherein [symbol] indicates the point of attachment of the ring to L_{A} or L_{B} and * indicates the point of attachment to the β-D-2′-deoxyribofuranosyl unit I′ of formula I or II.

In one embodiment In is 2′-deoxyzebularine (dZ).

In one embodiment In is 5-fluoro-2′-deoxyzebularine (FdZ).

In one embodiment X₋₁ is absent.

In one embodiment X₊₁ is absent. In one embodiment X₋₁ and X₋₂ are absent. In one embodiment X₊₁ and X₊₂ are absent. In one embodiment X₊₁ and X₋₁ are absent. In one embodiment X₋₁, X₋₂ and X₊₁ are all absent. In one embodiment X₊₁, X₊₂ and X₋₁ are all absent. In one embodiment X₋₁, X₋₂, X₊₁ and X₊₂ are all absent. In one embodiment Z_{A}, Z_{B}, and one of X₋₆, X₋₅, X₋₄, X₊₆, X₊₅, X₊₄, X₊₁, X₊₂, X₋₁ or X₋₂ are absent. In one embodiment Z_{A}, Z_{B}, and two of X₋₆, X₋₅, X₋₄, X₊₆, X₊₅, X₊₄, X₊₁, X₊₂, X₋₁ or X₋₂ are absent. In one embodiment Z_{A}, Z_{B}, and three of X₋₆, X₋₅, X₋₄, X₊₆, X₊₅, X₊₄, X₊₁, X₊₂, X₋₁ or X₋₂ are absent. In one embodiment Z_{A}, Z_{B}, and four of X₋₆, X₋₅, X₋₄, X₊₆, X₊₅, X₊₄, X₊₁, X₊₂, X₋₁ or X₋₂ are absent. In one embodiment Z_{A}, Z_{B}, and five of X₋₆, X₋₅, X₋₄, X₊₆, X₊₅, X₊₄, X₊₁, X₊₂, X₋₁ or X₋₂ are absent. In one embodiment Z_{A}, Z_{B}, and six of X₋₆, X₋₅, X₋₄, X₊₆, X₊₅, X₊₄, X₊₁, X₊₂, X₋₁ or X₋₂ are absent. In one embodiment Z_{A}, Z_{B}, and seven of X₋₆, X₋₅, X₋₄, X₊₆, X₊₅, X₊₄, X₊₁, X₊₂, X₋₁ or X₋₂ are absent. In one embodiment Z_{A}, Z_{B}, and eight of X₋₆, X₋₅, X₋₄, X₊₆, X₊₅, X₊₄, X₊₁, X₊₂, X₋₁ or X₋₂ are absent. In one embodiment Z_{A}, Z_{B}, and nine of X₋₆, X₋₅, X₋₄, X₊₆, X₊₅, X₊₄, X₊₁, X₊₂, X₋₁ or X₋₂ are absent. In one embodiment Z_{A}, Z_{B}, and all of X₋₆, X₋₅, X₋₄, X₊₆, X₊₅, X₊₄, X₊₁, X₊₂, X₋₁ or X₋₂ are absent. In one embodiment Z_{A}, Z_{B}, X₋₆, X₋₅, X₊₁, and X₊₂ are all absent. In one embodiment Z_{A}, Z_{B}, X₋₆, X₋₂, X₊₁, and X₊₂ are all absent. In one embodiment Z_{A}, Z_{B}, X₋₂, X₋₁, X₊₁, and X₊₂ are all absent. In one embodiment Z_{A}, Z_{B}, X₋₆, X₋₅, X₋₄, X₊₆, X₊₅, X₊₄, X₊₁, X₊₂ and X₋₁ are all absent. In one embodiment Z_{A}, Z_{B}, X₋₆, X₋₅, X₋₄, X₊₆, X₊₅, X₊₄, X₊₁, X₊₂, X₋₁ and X₋₂ are all absent.

In a third aspect the invention provides a compound of formula III

5′ X₋₆-X₋₅-X₋₄-dC^{N3}-X₋₂-X₋₁—In-X₊₁-X₊₂-dH^{E}-X₊₄-X₊₅ X₊₆ 3′ III

wherein X₋₆, X₋₅, X₋₄, X₋₂, X₋₁, X₊₁, X₊₂, X₊₄, X₊₅, X₊₆ and In are as defined for formula I; dC^{N3} is

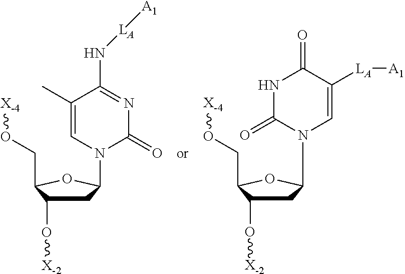

and dH^{E} is

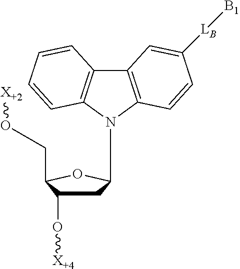

wherein A₁, B₁, L_{A} and L_{B} are as defined for formula I.

In a fourth aspect the invention provides a compound of formula IVa-d

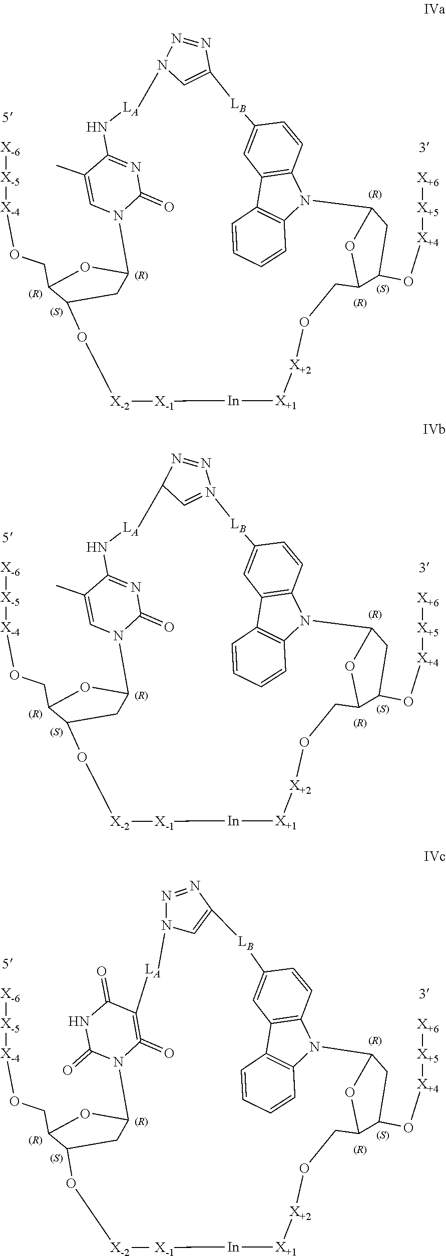

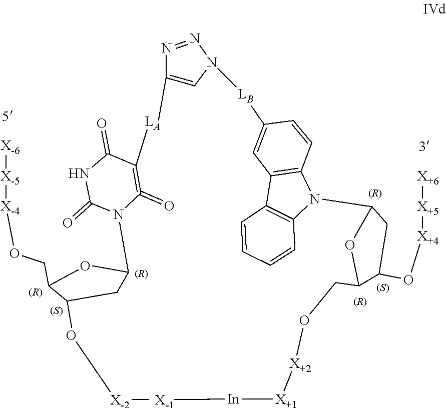

wherein L_{A}, L_{B}, X₋₆, X₋₅, X₋₄, X₋₂, X₋₁, X₊₁, X₊₂, X₊₄, X₊₅, X₊₆ and In are defined as for formula I.

In the third and fourth aspects:

In one embodiment L_{A} is —CH₂— or —CH₂CH₂—.

In one embodiment L_{B} is meta-ethynylbenzen-1-yl, or 5-hexynyl-1-yl or absent.

In one embodiment In is 2′-deoxyzebularine (dZ).

In one embodiment In is 5-fluoro-2′-deoxyzebularine (FdZ).

In one embodiment X₋₅ is 2′-deoxyadenosine and X₋₄, X₋₁, X₊₄, X₊₅ and X₊₆ are each thymidine and X₋₆, X₋₂, X₊₁ and X₊₂ are absent.

In one embodiment X₋₆ is 2′-deoxyadenosine and X₋₅, X₋₄, X₊₄, X₊₅ and X₊₆ are each thymidine and X₋₂, X₋₁, X₊₁ and X₊₂ are absent.

In one embodiment X₋₄ is 2′-deoxyadenosine and X₋₂, X₋₁, X₊₄, X₊₅ and X₊₆ are each thymidine and X₋₆, X₋₅, X₊₁ and X₊₂ are absent.

In one embodiment X₋₁ is thymidine and X₋₆, X₋₅, X₋₄, X₋₂, X₊₁, X₊₂, X₊₄, X₊₅, X₊₆, are all absent.

In one embodiment X₋₆, X₋₅, X₋₄, X₋₂, X₋₁, X₊₁, X₊₂, X₊₄, X₊₅, X₊₆ are all absent.

In a fifth aspect the invention provides a compound of formula V

5′ X₋₆-X₋₅-X₋₄-dU^{E}-X₋₂-X₋₁—In-X₊₁-X₊₂-dA^{N3}-X₊₄-X₊₅-X₊₆ 3 V

wherein X₋₆, X₋₅, X₋₄, X₋₂, X₋₁, X₊₁, X₊₂, X₊₄, X₊₅, X₊₆ and In are defined as for formula I;

dU^{E} is

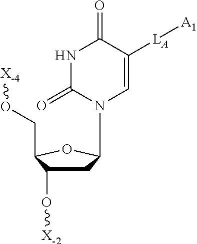

and dA^{N3} is

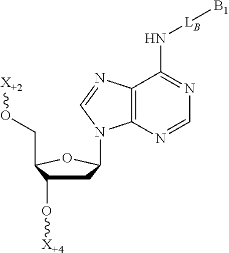

wherein A₁, B₁, L_{A} and L_{B} are as defined for formula I.

In one embodiment A₁ is —C≡CH.

In one embodiment B₁ is —N₃.

In a sixth aspect the invention provides a compound of formula VIa or VIb

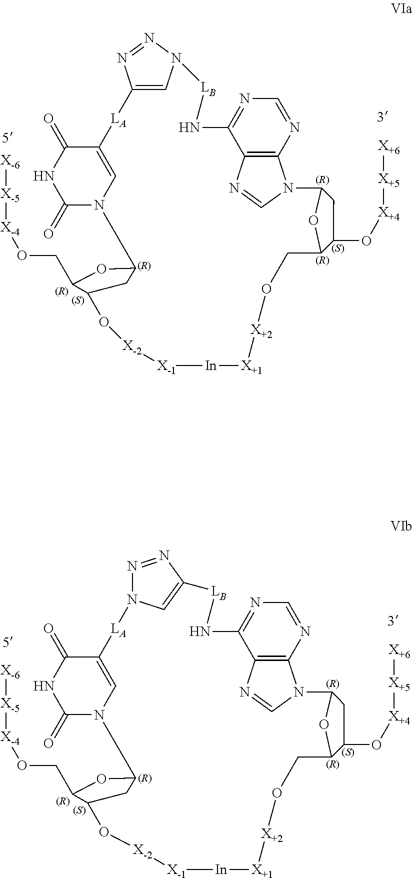

wherein L_{A}, L_{B}, X₋₆, X₋₅, X₋₄, X₋₂, X₋₁, X₊₁, X₊₂, X₊₄, X₊₅, X₊₆ and In are defined as for formula I.

In the fifth and sixth aspects:

In one embodiment L_{A} is —CH₂— or absent.

In one embodiment L_{B} is —CH₂CH₂—.

In one embodiment In is 2′-deoxyzebularine (dZ).

In one embodiment In is 5-fluoro-2′-deoxyzebularine (FdZ).

In one embodiment X₋₅ is 2′-deoxyadenosine and X₋₄, X₋₁, X₊₄, X₊₅ and X₊₆ are each thymidine and X₋₆, X₋₂, X₊₁ and X₊₂ are absent.

In one embodiment X₋₆ is 2′-deoxyadenosine and X₋₅, X₋₄, X₊₄, X₊₅ and X₊₆ are each thymidine and X₋₂, X₋₁, X₊₁ and X₊₂ are absent.

In one embodiment X₋₄ is 2′-deoxyadenosine and X₋₂, X₋₁, X₊₄, X₊₅ and X₊₆ are each thymidine and X₋₆, X₋₅, X₊₁ and X₊₂ are absent.

In one embodiment X₋₁ is thymidine and X₋₆, X₋₅, X₋₄, X₋₂, X₊₁, X₊₂, X₊₄, X₊₅, X₊₆, are all absent.

In one embodiment X₋₆, X₋₅, X₋₄, X₋₂, X₋₁, X₊₁, X₊₂, X₊₄, X₊₅, X₊₆ are all absent.

In a seventh aspect the invention provides a compound of formula VII

5′ X₋₆-X₋₅-X₋₄-dR^{NE}-X₋₂-X₋₁—In-X₊₁-X₊₂-dA^{Y}-X₊₄-X₊₅-X₊₆ 3 VII

wherein X₋₆, X₋₅, X₋₄, X₋₂, X₋₁, X₊₁, X₊₂, X₊₄, X₊₅, X₊₆ and In are defined as for formula I;

dR^{N3} is

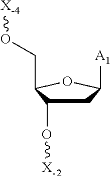

and dA^{Y} is

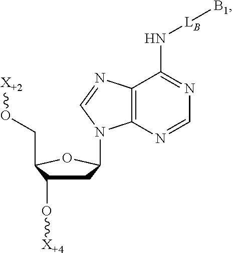

wherein A₁, B₁ and L_{B} are as defined for formula I.

In one embodiment A₁ is —N₃.

In one embodiment B₁ is —C≡CH.

In an eighth aspect the invention provides a compound of formula VIIIa or VIIIb

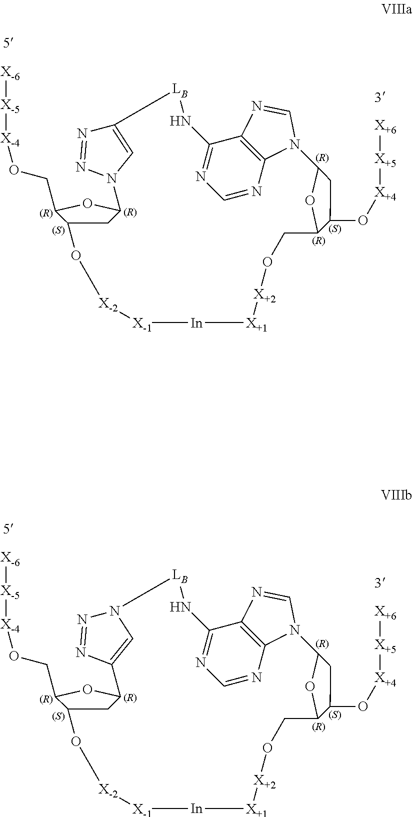

wherein L_{B}, X₋₆, X₋₅, X₋₄, X₋₂, X₋₁, X₊₁, X₊₂, X₊₄, X₊₅, X₊₆ and In are defined as for formula I.

In the seventh and eighth aspects:

In one embodiment L_{B} is —CH₂CH₂— or —CH₂CH₂CH₂CH₂—.

In one embodiment In is 2′-deoxyzebularine (dZ).

In one embodiment In is 5-fluoro-2′-deoxyzebularine (FdZ).

In one embodiment X₋₅ is 2′-deoxyadenosine and X₋₄, X₋₁, X₊₄, X₊₅ and X₊₆ are each thymidine and X₋₆, X₋₂, X₊₁ and X₊₂ are absent.

In one embodiment X₋₆ is 2′-deoxyadenosine and X₋₅, X₋₄, X₊₄, X₊₅ and X₊₆ are each thymidine and X₋₂, X₋₁, X₊₁ and X₊₂ are absent.

In one embodiment X₋₄ is 2′-deoxyadenosine and X₋₂, X₋₁, X₊₄, X₊₅ and X₊₆ are each thymidine and X₋₆, X₋₅, X₊₁ and X₊₂ are absent.

In one embodiment X₋₁ is thymidine and X₋₆, X₋₅, X₋₄, X₋₂, X₊₁, X₊₂, X₊₄, X₊₅, X₊₆, are all absent.

In one embodiment X₋₆, X₋₅, X₋₄, X₋₂, X₋₁, X₊₁, X₊₂, X₊₄, X₊₅, X₊₆ are all absent.

In a ninth aspect the invention provides a compound of formula IX

5′ Z_{A}—Xₖ—Wₗ—In—Yₙ—Z_{B} 3′ IX

wherein

W is a nucleotide or derivative thereof, or is absent and I is selected from 1-5;

Xₖ is a chain of nucleotides and/or derivatives thereof which are complementary or partially complementary to a chain of nucleotides and/or derivatives thereof Yₙ, wherein each of k and n is independently selected from 3-10; and

Z_{A}, Z_{B} and In are as defined in claim 1; and

the compound of formula IX forms a unimolecular hairpin in solution in which

W—In forms a loop and Xₖ and Yn form a stem.

In one embodiment Xₖ is a chain of nucleotides and/or derivatives thereof which are complementary to a chain of nucleotides or derivatives thereof Yₙ, wherein each of k and n is independently selected from 3-10.

In one embodiment, W is a native nucleotide. In one embodiment, Xₖ is a chain of native nucleotides which are complementary to a chain of native nucleotides Yn, wherein each of k and n is independently selected from 3-10.

In one embodiment In is 2′-deoxyzebularine (dZ).

In one embodiment In is 5-fluoro-2′-deoxyzebularine (FdZ).

In one embodiment In is 1-[2-deoxy-β-D-erythro-pentofuranosyl]-1,3,4,7-tetrahydro-2H-1,3-diazepin-2-one.

In one embodiment k=n and is an integer between 3 and 5 and I is an integer between 1 and 3.

In a tenth aspect the invention provides a compound of one of formulae X-XIII

5′ G-C-G-C-T-T-In-G-C-G-C 3′ X

5′ A-A-G-C-T-T-T-In-A-G-C-T-T 3′ XI

5′ T-G-C-G-C-T-T-In-G-C-G-C-T 3′ XII

5′ C-C-C-A-T-C-A-T-T-In-G-A-T-G-G-G 3′ XIII

wherein In is as defined for formula I.

In another aspect the invention provides a method for preparing a compound of formula IIa or IIb, the method comprising crosslinking a compound of formula I using Cu(I)-catalyzed azide-alkyne cycloaddition.

In another aspect the invention relates to a pharmaceutical composition comprising a compound of any one of formulae I to XIII and pharmaceutically acceptable carrier, diluent or excipient.

In another aspect the invention relates to a method for treating a disease or condition in a subject comprising administering to the subject a compound of any one of formulae I to XIII, wherein the disease or condition would be at least partially alleviated by inhibition of a human cytidine deaminase of the APOBEC3 family.

The invention may also be said broadly to consist in the parts, elements and features referred to or indicated in the specification of application, individually or collectively, in any or all combinations of two or more of said parts, elements or features, and where specific integers are mentioned herein that have known equivalents in the art to which the invention relates, such known equivalents are deemed to be incorporated herein as if individually set forth.

### 4. BRIEF DESCRIPTION OF THE FIGURES

The invention will now be described by way of example only and with reference to the drawings in which:

FIG. 1A is a scheme showing catalysis of the conversion of cytosine to uracil by APOBEC3 enzymes; FIG. 1B shows the structure of inhibitors dZ and FdZ incorporated into ssDNA; FIG. 1C shows the crystal structure of A3B_{CTD}-QM-ΔL3-AL1swap bound to ssDNA showing C⁰ flipped out to project into the Zn²⁺-containing active site. +1 2′-Deoxyadenosine and −2 thymidine are close together in the U-shaped conformation in ssDNA (PDB:5TD5).

FIG. 2 is a scheme showing the terminal (A, B) and internal (C—H) cross-linking strategies by Cu(I)-catalyzed azide-alkyne cycloaddition (CuAAC) used to pre-shape a linear ssDNA as a substrate (dC-containing oligo) and as an inhibitor of A3A and A3B (dZ or FdZ-containing oligo). (B) Alkyne (Y) and azide (dR^{N3}) modifications used in the sequence for terminal cross-linking strategy. (C, E and G) Internal cross-linking strategies as well as alkyne and azide modifications used for creation of a 1,4-disubstituted 1,2,3-triazole cross-link (D, F and H, respectively).

FIG. 3 is a diagram showing the methodology used for the design and evaluation of cross-linked substrates and inhibitors. This strategy was used to design, synthesize, and evaluate the first nanomolar inhibitors of A3 enzymes.

FIG. 4 is a schematic representation of dC and FdZ DNA hairpins.

FIG. 5 is a set of graphs showing the initial rate of A3B_{CTD}-QM-ΔL3-AL1swap catalyzed deamination of terminally (A), internally (B-D) cross-linked oligos and a dC-hairpin in comparison with the linear substrate. Substrate concentrations are 100 μM in (A and B), 800 μM in (C) and 400 μM in (D). Enzyme concentrations are 50 nM in (A and B) and 300 nM in (C) and 100 nM in (D). Buffers are 50 mM citrate-phosphate (pH 5.5 for A-C) and 50 mM Na-phosphate (pH 6.0 for D), 200 mM NaCl, 2 mM β-mercaptoethanol, 200 μM 4,4-dimethyl-4-silapentane-1-sulfonic acid (DSS) and 10% D₂O at 25° C. Error bars are estimated standard deviations from triplicate measurements.

FIG. 6. is a set of graphs showing inhibition of A3B_{CTD}-QM-ΔL3-AL1swap-catalyzed deamination of 5′-T₄CAT by dZ/FdZ-containing linear and cross-linked oligos. Initial rate of deamination was measured under the following experimental conditions: 400 μM of 5′-T₄CAT, 8 μM (A, B) or 4 μM (C) of dZ/FdZ-containing oligos, 300 nM (A, B) or 200 nM (C) of A3B_{CTD}-QM-ΔL3-AL1swap in a 50 mM sodium phosphate buffer (pH 6.0) containing 100 mM NaCl, 2.5 mM β-mercaptoethanol, 0.1 M EDTA, 50 μM 3-(trimethylsilyl)-2,2,3,3-tetradeuteropropionic acid (TSP) at 25° C. Error bars are estimated standard deviations from triplicate measurements.

FIG. 7 is a set of graphs showing inhibition of A3B_{CTD}-QM-ΔL3-AL1swap-catalyzed deamination of dC-hairpin by FdZ[C^{N3}(−2),H^{E1}(+1)]X in the continuous NMR assay. A) Schematic representation of A3B_{CTD}-QM-ΔL3-AL1swap-catalyzed deamination of dC-hairpin to dU-hairpin. B) dC-hairpin consumption over time in the absence of inhibitor; C) dC-hairpin consumption over time in the presence of FdZ[C^{N3}(−2),H^{E1}(+1)]X (100 nM). Experimental results are shown by crosses and the global regression using Lambert's W function is shown by a solid line in B) and C). Deamination of dC-hairpin over time was measured under the following experimental conditions: 400-700 μM of dC-hairpin, 20-27 nM of A3B_{CTD}-QM-ΔL3-AL1swap in a 50 mM Na⁺/K⁺ phosphate buffer (pH 6.0) containing 100 mM NaF, 1 mM TCEP, 10% D₂O and 100 μM 3-(trimethylsilyl)-2,2,3,3-tetradeuteropropionic acid (TSP) at 25° C.

FIG. 8 is a schematic representation of dC-hairpin-3 and dC-hairpin-4.

FIG. 9 shows the crystal structures of APOBEC3A(E72A) with dC-hairpin-3 (A) and dC-hairpin-4 (B). Protein structure is shown in ribbons, nucleic acid is depicted as sticks. Important nucleic acid and protein residues are labelled.

FIG. 10A shows the active site of wild-type APOBEC3A bound to FdZ-hairpin-1. Hydrated FdZ is shown as thick sticks, while some amino acid residues in the active site of APOBEC3A are shown as thin sticks. FIG. 10B illustrates the mechanism of conversion of dZ and FdZ into hydrated species in the active site of APOBEC3.

FIG. 11 shows the X-ray structure of 32R. Ellipsoids are drawn at a 50% probability level.

FIG. 12 is a graph showing the initial rate of human CDA-catalysed deamination of 2′-deoxycytidine (dC) in the absence (no inhibitor) and presence of dZ, 34R and 34S. Conditions: 100 μM dC as a substrate, using different concentrations of inhibitors, 27 nM human CDA in 20 mM Na-phosphate buffer, pH 6.0 supplemented with 100 mM NaCl at 25° C. Absorbance was measured at 286 nm at the start of deamination. Samples labelled as “preincubated” refer to experiments when the enzyme was preincubated for 5 min with inhibitors and then 100 μM dC was added.

FIG. 13 is a graph showing the inhibition of A3B_{CTD}-QM-ΔL3-AL1swap-catalyzed deamination of 5′-T₄CAT by linear oligos containing dZ, 34R, 34S and 46. Initial rate of deamination was measured under the following experimental conditions: 400 μM of 5′-T₄CAT, at oligo concentration as indicated, 300 nM of A3B_{CTD}-QM-ΔL3-AL1swap in a 50 mM sodium phosphate buffer (pH 6.0) containing 100 mM NaCl, 2.5 mM β-mercaptoethanol, 0.1 M EDTA, 50 μM 3-(trimethylsilyl)-2,2,3,3-tetradeuteropropionic acid (TSP) and 20% D₂O at 25° C. Error bars are estimated standard deviations from triplicate measurements.

FIG. 14 is a graph showing the percentage of intact oligos determined after enzymatic digestion at 37° C. using snake venom phosphodiesterase (phosphodiesterase I, 0.016 units/L, from Sigma) in 120 min. Experiments were repeated at least twice with 6% error for each point.

### 5. DETAILED DESCRIPTION OF THE INVENTION

### 5.1. Definitions and Abbreviations

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Whenever a range is given in the specification, for example, a temperature range, a time range, or a composition range, all intermediate ranges and subranges, as well as all individual values included in the ranges given are intended to be included in the disclosure. In the disclosure and the claims, “and/or” means additionally or alternatively. Moreover, any use of a term in the singular also encompasses plural forms.

As used herein the term “comprising” means “consisting at least in part of”. When interpreting each statement in this specification that includes the term “comprising”, features other than that or those prefaced by the term may also be present. Related terms such as “comprise” and “comprises” are to be interpreted in the same manner.

The term “consisting essentially of” and grammatical variations thereof as used herein means the specified materials or steps and those that do not materially affect the basic and novel characteristic(s) of the claimed invention.

The term “consisting of” and grammatical variations thereof as used herein means the specified materials or steps of the claimed invention, excluding any element, step, or ingredient not specified in the claim.

The term “about” as used herein means a reasonable amount of deviation of the modified term such that the end result is not significantly changed. For example, when applied to a value, the term should be construed as including a deviation of +/−5% of the value.

Where the term “optionally” is used, it is intended that the subsequent feature may or may not occur. As such, use of the term “optionally” includes instances where the feature is present, and also instances where the feature is not present. For example, a group “optionally substituted by one hydroxy group” includes groups with and without a hydroxy substituent.

The term “therapeutically effective amount” refers to an amount of a compound of the invention, which is effective to provide “therapy” in a subject, or to “treat” a disease or disorder in a subject.

The terms “treating” and “treatment” as used herein refer to dealing with a disease in order to entirely or partially relieve one, some or all of its symptoms, or to correct or compensate for the underlying pathology. The terms “treating” and “treatment” also include “prophylaxis” unless otherwise indicated. The terms “therapeutic” and “therapeutically” should be interpreted in a corresponding manner. Similarly, the term “treat” can be regarded as “applying therapy”.

The term “prophylaxis” includes primary prophylaxis to prevent the development of the disease and secondary prophylaxis whereby the disease has already developed, and the subject is temporarily or permanently protected against exacerbation of the disease or the development of new symptoms associated with the disease.

The term “subject” as used herein with reference to a method of treatment, refers to a warm-blooded animal to whom the treatment is applied. Examples of warm-blooded animals include, but are not limited to, primates, livestock animals (for example, sheep, cows, pigs, goats, horses) and companion animals (for example, cats and dogs). In one embodiment, the warm-blooded animal is a human.

Asymmetric centers may exist in the compounds described herein. The asymmetric centers may be designated as (R) or (S), depending on the configuration of substituents in three-dimensional space at the chiral carbon atom. All stereochemical isomeric forms of the compounds, including diastereomeric, enantiomeric, and epimeric forms, as well as D-isomers and L-isomers, and mixtures thereof, including enantiomerically enriched and diastereomerically enriched mixtures of stereochemical isomers, are within the scope of the invention.

Individual enantiomers can be prepared synthetically from commercially available enantiopure starting materials or by preparing enantiomeric mixtures and resolving the mixture into individual enantiomers. Resolution methods include conversion of the enantiomeric mixture into a mixture of diastereomers and separation of the diastereomers by, for example, recrystallization or chromatography, and any other appropriate methods known in the art. Starting materials of defined stereochemistry may be commercially available or made and, if necessary, resolved by techniques well known in the art.

The compounds described herein may also exist as conformational or geometric isomers, including cis, trans, syn, anti, entgegen (E), and zusammen (Z) isomers. All such isomers and any mixtures thereof are within the scope of the invention.

Also within the scope of the invention are any tautomeric isomers or mixtures thereof of the compounds described. As would be appreciated by those skilled in the art, a wide variety of functional groups and other structures may exhibit tautomerism. Examples include, but are not limited to, keto/enol, imine/enamine, and thioketone/enethiol tautomerism.

The compounds described herein may also exist as isotopologes and isotopomers, wherein one or more atoms in the compounds are replaced with different isotopes. Suitable isotopes include, for example, ¹H, ²H (D), ³H (T), ¹²C, ¹³C, ¹⁴C, ¹⁴N, ¹⁵N, ¹⁶O, and ¹⁸O. Procedures for incorporating such isotopes into the compounds described herein will be apparent to those skilled in the art. Isotopologes and isotopomers of the compounds described herein are also within the scope of the invention.

Also within the scope of the invention are salts of the compounds described herein, including pharmaceutically acceptable salts. Such salts include, acid addition salts, base addition salts, and quaternary salts of basic nitrogen-containing groups. Acid addition salts can be prepared by reacting compounds, in free base form, with inorganic or organic acids. Examples of inorganic acids include, but are not limited to, hydrochloric, hydrobromic, nitric, sulfuric, and phosphoric acid. Examples of organic acids include, but are not limited to, acetic, trifluoroacetic, propionic, succinic, glycolic, lactic, malic, tartaric, citric, ascorbic, maleic, fumaric, pyruvic, aspartic, glutamic, stearic, salicylic, methanesulfonic, benzenesulfonic, isethionic, sulfanilic, adipic, butyric, and pivalic. Base addition salts can be prepared by reacting compounds, in free acid form, with inorganic or organic bases. Examples of inorganic base addition salts include alkali metal salts, alkaline earth metal salts, and other physiologically acceptable metal salts, for example, aluminum, calcium, lithium, magnesium, potassium, sodium, or zinc salts. Examples of organic base addition salts include amine salts, for example, salts of trimethylamine, diethylamine, ethanolamine, diethanolamine, and ethylenediamine. Quaternary salts of basic nitrogen-containing groups in the compounds may be prepared by, for example, reacting the compounds with alkyl halides such as methyl, ethyl, propyl, and butyl chlorides, bromides, and iodides, dialkyl sulfates such as dimethyl, diethyl, dibutyl, and diamyl sulfates, and the like.

The term “pharmaceutically acceptable” is used to specify that an object (for example a salt, dosage form, diluent or carrier) is suitable for administration to a subject, in particular, a human subject. An example list of pharmaceutically acceptable salts can be found in the Handbook of Pharmaceutical Salts: Properties, Selection and Use, P. H. Stahl and C. G. Wermuth, editors, Weinheim/Zurich:Wiley-VCH/VHCA, 2002.

A suitable pharmaceutically acceptable salt of a compound of Formula (I) is, for example, an acid-addition salt. An acid-addition salt of a compound of Formula (I) may be formed by bringing the compound into contact with a suitable inorganic or organic acid under conditions known to the skilled person. An acid addition salt may for example be formed using an inorganic acid selected from the group consisting of hydrochloric acid, hydrobromic acid, sulfuric acid and phosphoric acid. An acid addition salt may also be formed using an organic acid selected from the group consisting of trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid and para-toluenesulfonic acid.

The compounds described herein may form or exist as solvates with various solvents. If the solvent is water, the solvate may be referred to as a hydrate, for example, a mono-hydrate, a di-hydrate, or a tri-hydrate. All solvated forms and unsolvated forms of the compounds described herein are within the scope of the invention.

The general chemical terms used herein have their usual meanings. Standard abbreviations for chemical groups are well known in the art and take their usual meaning, eg, Me=methyl, Et=ethyl, iPr=isopropyl, Bu=butyl, t-Bu=tert-butyl, Ph=phenyl, Bn=benzyl, DMT=4,4′-dimethoxytrityl, TIPS=triisopropylsilyl, TMS=trimethylsilyl, Tol=4-methylbenzoyl, and the like.

The term “nucleobase”, as used herein, includes naturally occurring nucleobases (for example, adenine, guanine, cytosine, thymine, uracil, inosine, 5-methylcytosine, 5-hydroxymethylcytosine, 5-formylcytosine, N⁶-methyladenosine, 8-oxoguanine and 8-oxoadenine) as well as non-natural nucleobases and modified variants thereof. Examples of non-natural nucleobases include but are not limited to 8-bromoguanine, 8-bromoadenine, 8-bromoinosine, 2,6-diaminopurine, 2-thio-thymine, 5-carboxamide-uracil, 3-deaza-adenine and 7-deaza-guanine.

A person of ordinary skill in the art will recognize that “nucleobase” encompasses purine and pyrimidine derivatives, as well as heterocyclic derivatives and tautomers thereof. See for example, “Nucleic acids in chemistry and biology”, Ed.: G. M. Blackburn, M. J. Gait, D. Loakes, D. W. Williams, 3^{rd} edition, RSC, 2006 and “Current Protocols in Nucleic Acid Chemistry”, Ed.: S. L. Beaucage, D. E. Bergstrom, P. Herdewijn, A. Matsuda, Wiley.

The term “nucleoside” as used herein refers to a glycoside of a heterocyclic “nucleobase”. The term “nucleoside” includes naturally occurring and non-naturally occurring nucleosides, as well as other nucleoside analogues. In one embodiment the term nucleoside comprises ribonucleosides (comprising a ribose moiety) and deoxyribonucleosides (comprising a 2-deoxyribose moiety).

The term “nucleotide” as used herein refers to a phosphorylated nucleoside. Nucleotides are the molecular building-blocks of DNA and RNA. Nucleotides include nucleoside monophosphates as well as nucleoside phosphorothioates and phosphoramidates. In one embodiment a nucleotide is a nucleoside monophosphate.

The term “native nucleotide”, as used herein means a nucleotide selected from thymidine, adenosine, guanosine, cytidine, uridine, 2′-deoxyadenosine, 2′-deoxyguanosine, 2′-deoxyuridine and 2′-deoxycytidine which can be a part of native DNA chain or RNA chain or “oligonucleotide”, also referred to as an “oligo”.

The terms “oligonucleotide” and “an oligo” as used herein, refer to a plurality of nucleotides and/or nucleotide derivatives joined by native phosphodiester bonds. An oligonucleotide is a polynucleotide of between at least 2 and about 300 nucleotides in length.

The term “nucleotide derivative” as used herein means a nucleotide-like compound that contains (a) an altered sugar moiety, and/or (b) a non-natural nucleobase and/or (c) a non-natural phosphate group.

Classical examples of nucleotide derivatives with non-natural sugars are locked nucleic acid (LNA), unlocked nucleic acid (UNA), 2′-OMe-RNA, 2′-F-RNA, 2′-methoxyethyl-RNA, hexitol nucleic acid (HNA), morpholino nucleic acid (MNA), arabino nucleic acid (ANA), 2′-fluoroarabino nucleic acid (FANA), glycol nucleic acid (GNA), threose nucleic acid (TNA), tricyclo-DNA, bicyclo[4.3.0]-DNA, bicyclo[3.2.1]-DNA and others.

Classical examples of modifications of non-natural phosphate groups in nucleic acids are those giving rise to phosphorothioate (PS), phosphorodithioate (PS2), triazole-linked DNA, methylated phosphate (POMe), mesyl phosphoramidate, phosphoryl guanidine, and others.

A person skilled in the art would be able to combine one or more derivatives of nucleotides in one oligonucleotide to potentially gain beneficial properties such as increased thermal stability, greater resistance against exo- and endo-nucleases, enhanced cell uptake and/or organ distribution and others.

The term “conjuvant” as used herein refers to a compound or drug that is administered in conjunction with another compound or drug, to prolong efficacy of the first compound or drug by delaying development of drug resistance and/or cancer metastasis.

The terms “halo”, “halide” or “halogen group” used herein refer to a fluoro, chloro, bromo or iodo group.

The term “alkyl” as used herein refers to refers to a saturated straight or branched acyclic hydrocarbon group, such as a straight or branched group of 1-20, 1-8, or 1-6 carbon atoms, referred to herein as (C₁-C₂₀)alkyl, (C₁-C₈)alkyl, and (C₁-C₆)alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, and the like.

The term “alkenyl” as used herein refers to an unsaturated straight or branched acyclic hydrocarbon group having at least one carbon-carbon double bond, such as a straight or branched group of 2-20, 2-8, or 2-6 carbon atoms, referred to herein as (C₂-C₂₀)alkenyl, (C₂-C₈)alkenyl, and (C₂-C₆)alkenyl, respectively. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, and 4-(2-methyl-3-butene)-pentenyl.

The term “cycloalkyl” as used herein refers to a saturated hydrocarbon ring group. The prefix “Cₓ-C_{y}”, wherein x and y are each an integer, when used in combination with the term “cycloalkyl” refers to the number of ring carbon atoms in the cycloalkyl group. Examples of cycloalkyl groups include cyclopropyl, cyclobutyl, cyclopentyl, and cyclohexyl as well as bridged and caged saturated ring groups such as, for example, adamantane.

The term “heterocycloalkyl” refers to a single aliphatic ring, containing at least 2 carbon atoms in addition to 1-3 heteroatoms independently selected from oxygen, sulfur, and nitrogen, as well as combinations comprising at least one of the foregoing heteroatoms. The prefix “Cₓ-C_{y}”, wherein x and y are each an integer, when used in combination with the term “heterocycloalkyl” refers to the number of ring carbon atoms in the heterocycloalkyl group. Suitable heterocycloalkyl groups include, for example (as numbered from the linkage position assigned priority 1), 2-pyrrolinyl, 2,4-imidazolidinyl, 2,3-pyrazolidinyl, 2-piperidyl, 3-piperidyl, 4-piperidyl, and 2,5-piperazinyl. Morpholinyl groups are also contemplated, including 2-morpholinyl and 3-morpholinyl (numbered wherein the oxygen is assigned priority 1). Substituted heterocycloalkyl also includes ring systems substituted with one or more oxo moieties, such as piperidinyl N-oxide, morpholinyl-N-oxide, 1-oxo-1-thiomorpholinyl and 1,1-dioxo-1-thiomorpholinyl.

The term “aryl” as used herein refers to a cyclic aromatic hydrocarbon group that does not contain any ring heteroatoms. Aryl groups include monocyclic and bicyclic ring systems. Examples of aryl groups include, but are not limited to, phenyl, azulenyl, heptalenyl, indenyl, indanyl, pentalenyl, and naphthyl. In some embodiments, aryl groups have from 6 to 20, 6 to 14, 6 to 12, or 6 to 10 carbon atoms in the ring(s). In some embodiments, the aryl groups are phenyl or naphthyl. Aryl groups include aromatic-carbocycle fused ring systems. Examples include, but are not limited to, indanyl and tetrahydronaphthyl. The prefix “Cₓ-C_{y}”, wherein x and y are each an integer, when used in combination with the term “aryl” refers to the number of ring carbon atoms in the aryl group. In some embodiments, “aryl” groups may be substituted with one or more optional substituents as described herein.

The term “heteroaryl”, as used herein refers to a radical of a monocyclic or polycyclic (e.g., bicyclic or tricyclic) 4n+2 aromatic ring system (e.g., having 6, 10, or 14 n electrons shared in a cyclic array) having ring carbon atoms and 1-4 ring heteroatoms provided in the aromatic ring system, wherein each heteroatom is independently selected from nitrogen, oxygen, and sulfur. A heteroaryl group is a variety of heterocyclic group that possesses an aromatic electronic structure.

In heteroaryl groups that contain one or more nitrogen atoms, the point(s) of attachment can be carbon or nitrogen atoms, as valency permits. Heteroaryl polycyclic ring systems can include one or more heteroatoms in one or both rings. The term “heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl. Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepanyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, OP(═O)(NR^{bb})₂, NR^{bb}P(═O)(OR^{cc})₂, NR^{bb}P(═O)(NR^{bb})₂, —P(R^{cc})₂, —P(R^{cc})₃, —OP(R^{cc})₂, OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₄ carbocyclyl, 3-14 membered heterocyclyl, C₆₋₁₄aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, =═NNR^{bb}S(═O)₂R^{aa}, ═NR^{bb}, or ═NOR^{aa};
each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{aa})OR^{aa}, —C(═NR^{cc})N(R^{cc})₂, —SO₂N(R^{CC})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R¹)₂, —N(R^{f})₂, —N(R^{f})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, NRC(═O)R^{ee}, NR^{ff}CO₂R^{ee}, NR^{ff}C(═O)N(R^{ff})₂, C(═NR^{ff})OR^{ee}, OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, NR^{ff}SOR^{ee}, —SO₂N(R^{ff})₂, —SO₂R^{ee}, —SO₂OR^{ee}, —OSO₂R^{ee}, S(═O)R^{ee}, —Si(R^{ee})₃, OSi(R^{ee})₃, —C(═S)N(R^{ff})₂, —C(═O)SR^{ee}, —C(═S)SR^{ee}, SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆-10 aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₄ perhaloalkyl, C₂₋₆ alkenyl, C₂₋₆ alkynyl, C₃₋₁₀carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆ to aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₄ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₄ alkyl), —OC(═NH)(C₁₋₄ alkyl), —OC(═NH)OC₁₋₄ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₄ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₄ alkyl, —OSO₂C₁₋₆ alkyl, —SOC₁₋₄ alkyl, —Si(C₁₋₄ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₄ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X is a counterion.

As used herein, the terms “hydroxyl” and “hydroxy” refers to the group —OH. The terms “substituted hydroxyl” or “substituted hydroxyl,” by extension, refer to a hydroxyl group wherein the oxygen atom directly attached to the parent molecule is substituted with a group other than hydrogen, and includes groups selected from OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein.

As used herein, the term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═O)SR^{aa}, —SC(═O)OR^{aa}, and —SC(═O)R^{aa}, wherein R^{aa}, and R^{cc} are as defined herein.

As used herein, the term, “amino” refers to the group —NH₂. The term “substituted amino,” by extension, refers to a monosubstituted amino, a disubstituted amino, or a trisubstituted amino, as defined herein.

As used herein, the term “monosubstituted amino” refers to an amino group wherein the nitrogen atom directly attached to the parent molecule is substituted with one hydrogen and one group other than hydrogen, and includes groups selected from —NH(R^{bb}), NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(NR^{bb})₂, wherein R^{aa}, R^{bb}, and R^{cc} are as defined herein, and wherein R^{bb} of the group —NH(R^{bb}) is not hydrogen.

As used herein, the term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and NR^{bb}P(═O)(NR^{bb})₂, wherein R^{aa}, R^{bb}, and R^{cc} are as defined herein, with the proviso that the nitrogen atom directly attached to the parent molecule is not substituted with hydrogen.

As used herein, the term “trisubstituted amino” or a “quaternary amino salt” or a “quaternary salt” refers to a nitrogen atom covalently attached to four groups such that the nitrogen is cationic, wherein the cationic nitrogen atom is further complexed with an anionic counterion, e.g., such as groups of the formula N(R^{bb})₃⁺X⁻ and N(R^{bb})₂—⁺X⁻, wherein R^{bb} and X are as defined herein.

As used herein, a “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

As used herein, the term “sulfonyl” refers to a group selected from —SO₂N(R^{bb})₂, —SO₂R^{aa}, and —SO₂OR^{aa}, wherein R^{aa} and R^{bb} are as defined herein.

As used herein, the term “sulfinyl” refers to the group S(═O)R^{aa}, wherein R^{aa} is as defined herein.

As used herein, the term “acyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}), thioesters (—C(═O)SR^{aa}, —C(═S)SR^{aa}), amides (—C(═O)N(R^{bb})₂, —C(═O)NR^{bb}SO₂R^{aa}) thioamides (—C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

As used herein, the term “azido” refers to a group of the formula: —N₃.

As used herein, the term “cyano” refers to a group of the formula: —CN.

As used herein, the term “isocyano” refers to a group of the formula: —NC.

As used herein, the term “nitro” refers to a group of the formula: —NO₂.

As used herein, the term “halo” or “halogen” refers to fluorine (fluoro, —F), chlorine (chloro, —Cl), bromine (bromo, —Br), or iodine (iodo, —I).

As used herein, the term “oxo” refers to a group of the formula: ═O.

As used herein, the term “thiooxo” refers to a group of the formula: ═S.

As used herein, the term “imino” refers to a group of the formula: ═N(R^{b}).

As used herein, the term “silyl” refers to the group —Si(R^{aa})₃, wherein R^{aa} is as defined herein.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, N(R^{aa})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

### 5.2. The Compounds of the Invention

The inventors have surprisingly determined that certain ssDNA oligonucleotides containing cytidine nucleoside analogues can be cross-linked to provide unexpectedly effective inhibitors of A3 family enzymes.

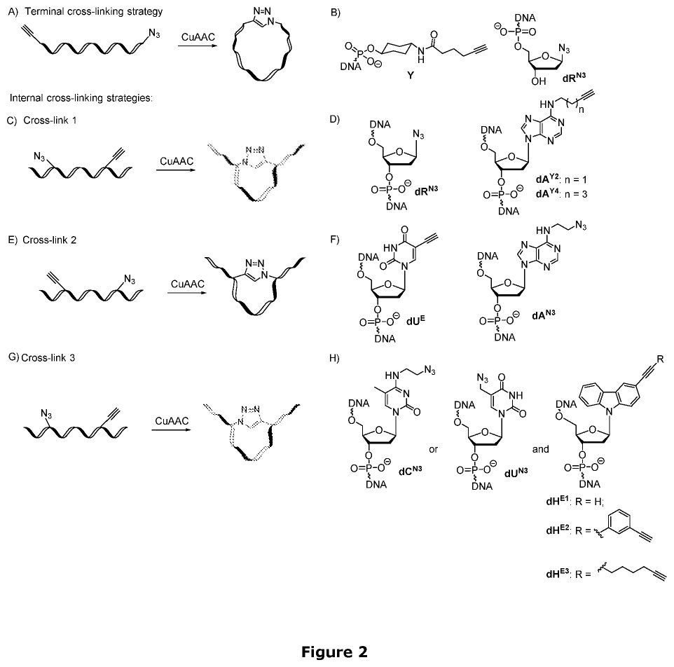

Two different approaches were explored for pre-shaping ssDNA oligonucleotides using covalent cross-links between non-contiguous nucleotides in a ssDNA oligonucleotide sequence, i.e. terminal and internal cross-linking strategies (FIG. 2). In addition to flexibility in the positioning of the crosslink with respect to the inhibitor unit, the length of the ssDNA oligonucleotide inhibitor can be reduced to the minimum, providing cross-linked 3-mer and 4-mer ssDNA oligonucleotide inhibitors with Kᵢ below 1 μM.

The compounds of the invention include linear ssDNA oligonucleotides incorporating (a) an azide-modified heteroaryl group attached to a β-D-2′-deoxyribofuranosyl unit, (b) a terminal alkyne modified heteroaryl group attached to a β-D-2′-deoxyribofuranosyl unit and (c) a 2′-deoxynucleoside form of an inhibitor of cytidine deaminase incorporated into the oligodeoxynucleotide in place of 2′-deoxycytidine, as represented by formula I above.

The compounds of the invention also include cross-linked ssDNA oligonucleotides in which the linear ssDNA oligonucleotides described above have undergone azide-alkyne cycloaddition, as represented by formulae ha and IIb. The cross-linking of the ssDNA oligonucleotides was carried out using copper(I)-catalyzed azide-alkyne cycloaddition (CuAAC). CuAAC was chosen because it is compatible with DNA synthesis and does not change the chemical structure of the nucleotides.

The compounds of the invention may be prepared using the methods and procedures described herein or methods and procedures analogous thereto. Other suitable methods for preparing compounds of the invention will be apparent to those skilled in the art.

It will be appreciated that where typical or preferred process conditions (for example, reaction temperatures, times, mole ratios of reactants, solvents, pressures, etc.) are indicated, other process conditions can also be used unless otherwise stated. Optimum reaction conditions may vary with the particular reactants used.

The starting materials useful in the methods and reactions are commercially available or can be prepared by known procedures or modifications thereof, for example those described in standard reference texts⁵⁷⁻⁶⁰.

The various starting materials, intermediates, and compounds may be isolated and purified where appropriate using conventional techniques such as precipitation, filtration, crystallization, evaporation, distillation, and chromatography. Characterization of the compounds may be performed using conventional methods such as by melting point, mass spectrum, nuclear magnetic resonance, and various other spectroscopic analyses.

Conventional protecting groups may be necessary to prevent certain functional groups from undergoing undesired reactions. The need for protection and deprotection and the selection of appropriate protecting groups can be readily determined by a person skilled in the art. Suitable protecting groups for various functional groups as well as suitable conditions for protecting and deprotecting particular functional groups are well known in the art (see, for example,⁶¹)

The first step in manufacturing the compounds of the invention is to obtain or synthesize the appropriate DNA precursors containing the azide or alkyne group. Example 1 sets out the synthesis of DNA phosphoramidites containing a terminal alkyne or azide group.

Other chemistry for the synthesis of oligonucleotides can be employed such as H-phosphonate, phosphodiester, phosphotriester or phosphite triester methods which are well-known in the art⁶²⁻⁶³ provided that stable DNA precursors containing terminal azide and alkyne groups can be obtained using these methods.

In the last 15 years, alkyne- and azide-modified ssDNA oligonucleotides have been widely used for the synthesis of a vast variety of bioconjugates via azide-alkyne ‘click chemistry’. Usually, the terminal alkynes required for ‘click chemistry’ can be introduced into ssDNA oligonucleotides using phosphoramidite chemistry. Several reagents used in the synthesis of terminal alkynes are commercially available, such as the Y and dU^{E} compounds (FIG. 2) used in the current work. However, there are limited options available for the introduction of the azide functionality into ssDNA oligonucleotides, especially in the middle of the DNA sequence. The work disclosed herein uses azide-carrying phosphoramidites to produce ssDNA oligonucleotides having internal organic azide modifications.

As described in the Examples, ssDNA oligonucleotides containing an organic azide at the 3′-end as well as in the middle of the sequence using azide-modified 2-deoxy-D-ribose, dA and dC were synthesized according to the two approaches detailed herein and summarised in FIG. 2.

In the first approach (terminal cross-linking), terminal alkyne and azide moieties were incorporated at 5′ and 3′-ends of ssDNA oligonucleotides, respectively. Commercially available alkyne Y (FIG. 2B) was used and in-house synthesized azido-sugar dR^{N3} was attached to the solid support (controlled-pore glass, CPG). ssDNAs of different lengths were synthesized by standard DNA phosphoramidite chemistry.

In the second approach (internal cross-linking), in-house synthesized 2′-deoxyadenosine derivatives (dA^{Y2}, dA^{Y4}, dA^{N3}) or N-carbazolyl nucleosides (dH^{E1}, dH^{E2}, dH^{E3}) were used in position +1 and commercially available phosphoramidite of 5-ethynyl-2′-deoxyuridine⁵ (dU^{E}, FIG. 2F) or in-house synthesized phosphoramidites of azido-sugar dRN³ or modified 2′-deoxycytidine (dC^{N3}) or 2′-deoxyuridine (dU^{N3}) were used in position −2.

In both approaches, CuAAC cross-linking was carried out as a post-synthetic step, monitored by analytical reverse-phase HPLC and mass spectrometry.

Various synthetic techniques that have been described can be used for the synthesis of DNA precursors containing terminal alkyne and azide groups as exemplified by Fantoni et al.⁶⁴ and El-Sagheer et al.⁶⁵

In Example 1, azide and terminal alkyne containing linkers were introduced into purines and pyrimidines by alkylation of suitably protected native nucleosides. A terminal alkyne can be also introduced using the Sonogashira reaction starting from halogenated purine or pyrimidine nucleosides or from halogenated pseudo-nucleosides containing a heterocycle different from purine or pyrimidine as shown in Example 1. Such nucleosides can also bear a suitable protecting group in the structure. Pseudo-nucleosides containing a heterocycle different from native purines or pyrimidines can be prepared from Hoffer's chlorosugar 1 and a heterocycle in a substitution reaction usually in the presence of a base as shown in Example 1 for iodo-carbazole derivative 20. Other methods for obtaining N- and C-nucleosides are known⁶³. Once the 2′-deoxynucleoside carrying azide or terminal alkyne is formed, it can be further converted to the DNA precursor by introducing protecting groups on the nucleobase (if needed) followed by introduction of 5′-O-DMT protecting group and 3′-O-phosphitylation as shown in typical protocols in Example 1. One should note that protocols of phosphitylation of compounds containing terminal alkyne and organic azide are different as shown in Example 1.

Once the desired phosphoramidites have been produced, they can be incorporated into ssDNA oligonucleotides using standard oligonucleotide synthesis methods, as described in Example 2. The positioning of the azide and alkyne-containing nucleotides in the oligonucleotide sequence is important for creation of the cross-link. The oligonucleotides of the invention also incorporate a 2′-deoxynucleoside form of an inhibitor of cytidine deaminase (In). This unit is incorporated into the linear oligonucleotide chain in place of 2′-deoxycytidine.

It is essential that the cross-link present in the final oligonucleotide sequence is positioned close to the In unit (i.e. within +1 . . . +6 and −1 . . . −6 positions). In the preferred sequence, the cross-link connects positions −2 and +1, −1 and +1.

A person skilled in the art will recognize that such inhibitors can be obtained by Hilbert-Johnson reaction or a silyl variation of it using Hoffer's chloro-sugar 1 and modified nucleobase (silylated or not) as described in the literature⁶³ and exemplified by the synthesis of 2′-deoxy-D-ribose derivatives of zebularine and 5-fluorozebularine,⁶⁻⁹ dZ and FdZ, respectively.¹⁻² A similar procedure was used in the past for preparation of ribose versions of CDA inhibitors.¹⁰⁻¹⁶ Once the 2′-deoxynucleoside is formed, it can be further converted to the DNA precursor by introducing protecting groups on the nucleobase (if needed) followed by introduction of 5′-O-DMT protecting group and 3′-O-phosphitylation as shown in typical protocols in Example 1.

Once the ssDNA oligonucleotide is prepared, it is cross-linked using copper(I)-catalyzed azide alkyne cycloaddition, as described in Example 3.

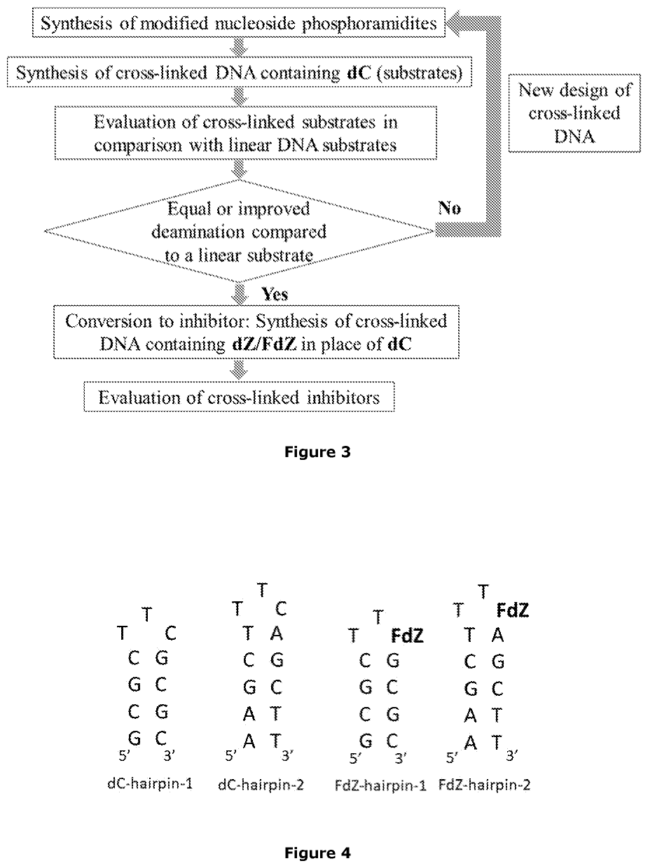

The cross-linked oligonucleotides (oligos) produced were evaluated as A3 substrates and compared to a standard linear substrate by an NMR-based assay, as described in Example 4. The evaluation and optimization strategy employed is summarized in FIG. 3.

Real-time NMR assays are advantageous because they are direct, utilizing only A3 enzymes and oligos in a suitable buffer system, in contrast to many fluorescence-based assays where a secondary enzyme and a fluorescently modified oligo are used. The NMR-based assay described herein allows a Michaelis-Menten kinetic model to be used to characterize both A3 substrates and inhibitors based on direct determination of the initial velocity of deamination of various ssDNA substrates (including modified substrates) in the presence of A3 enzymes.

The A3 enzyme used in the experiments detailed herein is the well-characterized and very active A3B_{CTD}-QM-ΔL3-AL1swap,¹ where loop 3 is deleted and loop 1 is replaced with the corresponding loop 1 from A3A in addition to four point mutations remote to the enzyme's active site. Inventors also used active wild-type APOBEC3A to confirm that claimed compounds inhibit wild-type APOBEC3 enzyme. Recent work by the inventors on the related enzyme, A3G, has shown that inhibition of the C-terminal catalytic domain is mirrored by the inhibition of the full-length enzyme, validating their approach of using catalytic C-terminal domains for inhibitor development.²

The inhibitory potential of FdZ/dZ-containing ssDNA oligonucleotides was evaluated by the NMR-based assay in the presence of a linear substrate, as described in Example 5. The inventors also characterized the thermodynamics of binding of some cross-linked oligos with A3 enzymes in comparison with linear ssDNA by isothermal titration calorimetry (ITC). Specifically, the inactive E72A mutant of A3A was chosen to evaluate substrate binding and the active A3B_{CTD}-QM-ΔL3-AL1swap was chosen for inhibitor binding.

Based on the work detailed herein, the inventors believe that they are the first to identify and develop nM inhibitors of A3B_{CTD} by incorporating C-to-U inhibitors (exemplified by dZ and FdZ) in place of the target dC in cross-linked DNA fragments. The invention also provides compounds of formulae IX to XIII in which In is incorporated instead of dC in the loop of a DNA hairpin. A person skilled in the art would recognize that a cross-linked ssDNA can be mimicked by a DNA hairpin composed of a short stem and a loop. It has been shown that A3 enzymes also bind to and deaminate cytosine in short 3-4 nucleotide loops of DNA hairpins as such loops are still considered to be ssDNA.¹⁷⁻¹⁹ DNA hairpins of formulae IX to XIII can be synthesized using automated DNA synthesis by producing a ssDNA that self-assembles in a buffered aqueous solution, forming H-bonding interactions between complementary or partially complementary nucleotides present in the sequence.

The invention also provides a method for preparing a compound of formula IIa or IIb,

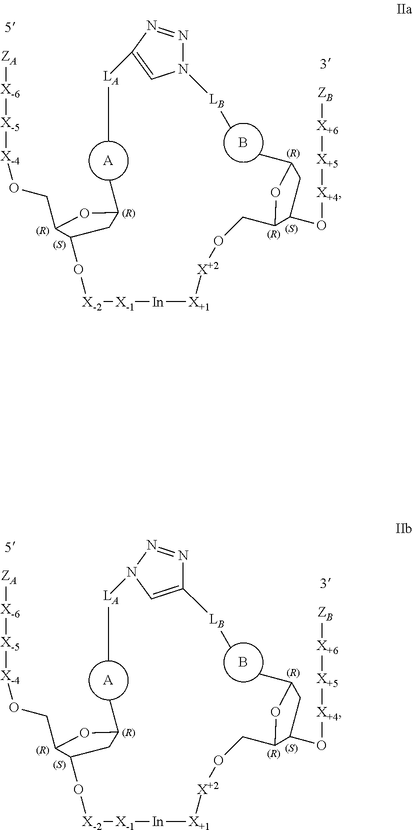

the method comprising crosslinking a compound of formula I

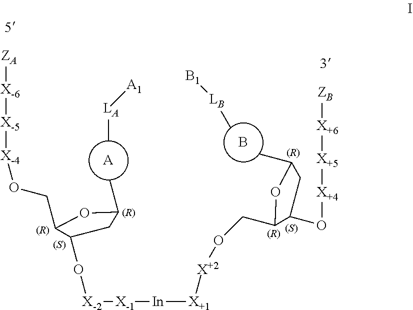

using Cu(I)-catalyzed azide-alkyne cycloaddition,
wherein L_{A}, L_{B}, A, B, A₁, B₁, X₋₆, X₋₅, X₋₄, X₋₂, X₋₁, X₊₁, X₊₂, X₊₄, X₊₅, X₊₆ and In are defined as for formula I above.

Whether the method results in a compound of formula IIa or IIb depends on which of A₁ and B₁ is the azide and which is the alkyne group.

As would be recognized by a person skilled in the art, the compound of formula IIa or IIb may comprise up to 12 nucleotides in addition to the inhibitor unit (In). In other embodiments, one or more, or even all of the nucleotides apart from those bearing the azide and alkyne groups, may be absent, leaving only a trimer oligonucleotide.

A person skilled in the art would recognise that chemical structures of the compounds of the invention can be presented in several ways. The structures below show an oligonucleotide of formula III that can be converted by Cu(I)-catalysed azide-alkyne cycloaddition to an oligonucleotide of formula IVb in which X₋₆, X₋₂, X₊₁ and X₊₂ are all absent, X₋₅ is 2′-deoxyadenosine, X₋₄, X₋₁, X₊₄, X₊₅ and X₊₆ are all thymidines, In is dZ or FdZ whereas dC^{N3} and dH^{E1} (FIG. 2H) are monomers present in formula III and form a 1,4-disubstituted 1,2,3-triazole cross-linked oligonucleotide IVb:

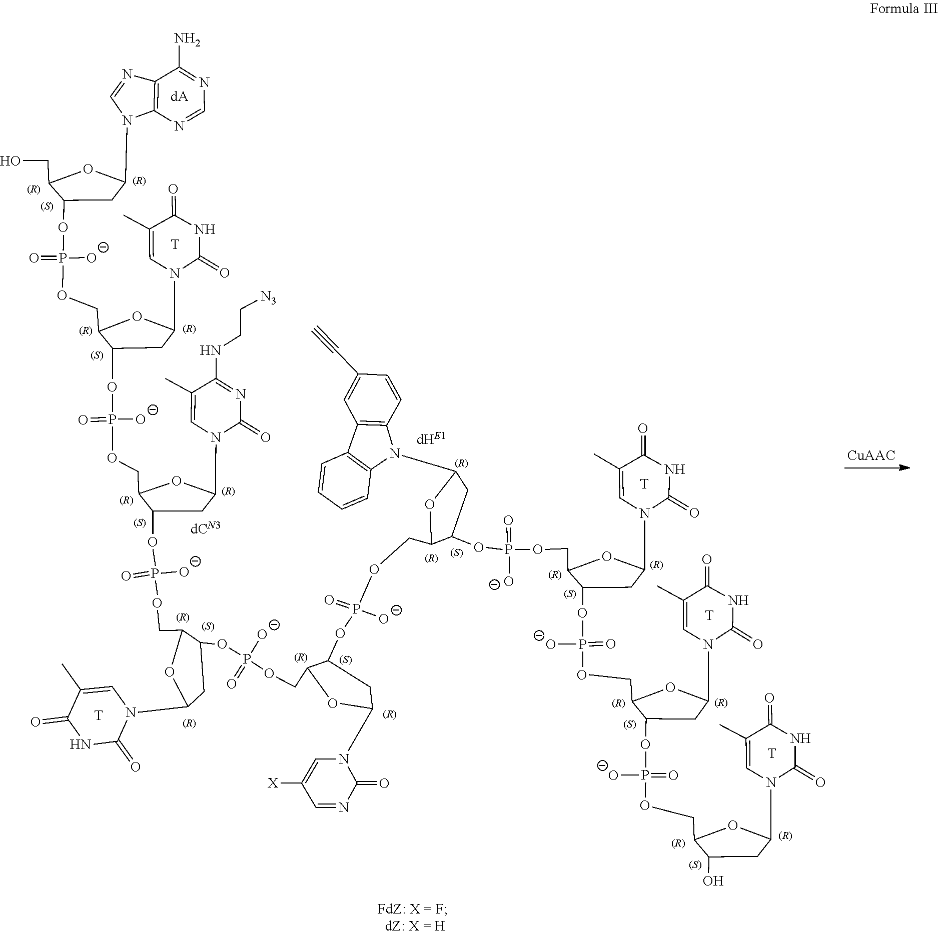

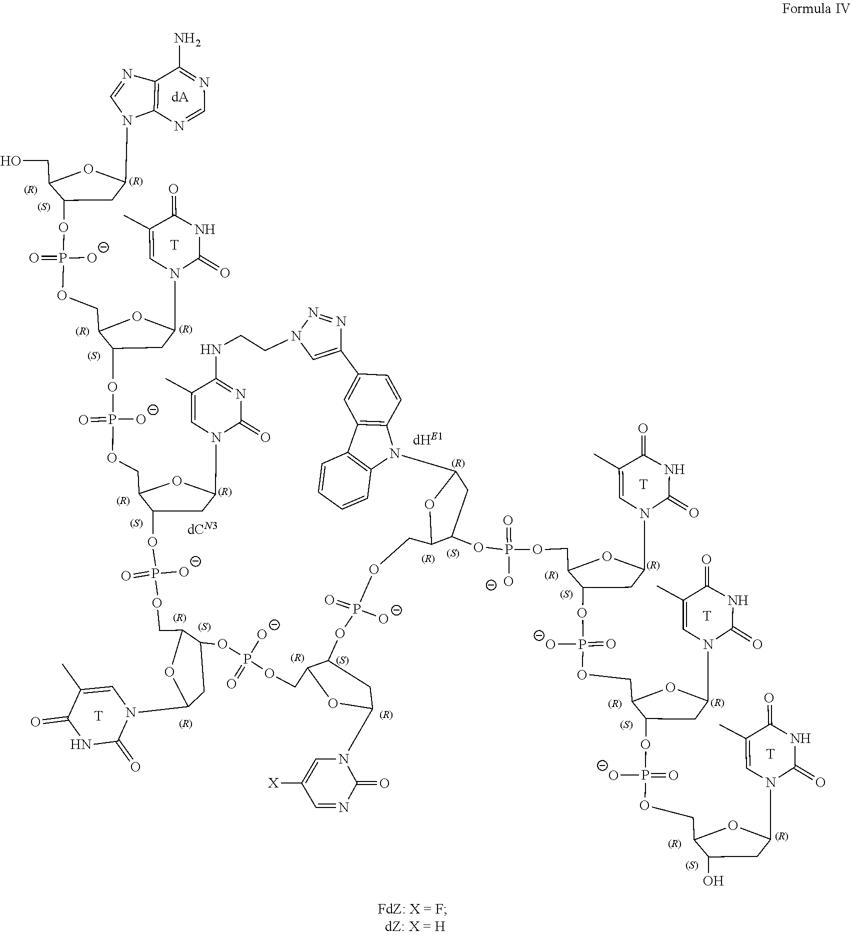

One can also simplify depiction of the latter cross-linked chemical structure by using abbreviations for the nucleotides and omitting the phosphodiester linkages; for example:

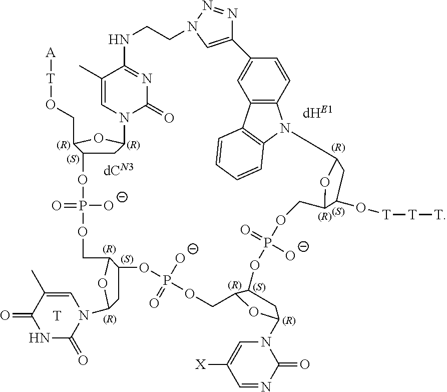

Further simplification provides the following depictions of compounds of the invention, where the nature of the cross link is clearly shown:

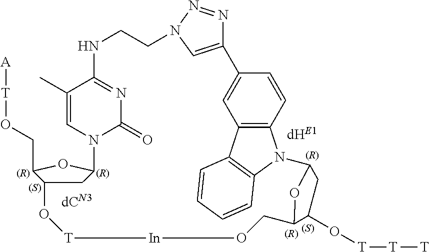

where In is dZ or FdZ.

These chemical structures can be conveniently described (using the definition provided in Example 3 in the caption of Table 4) as dZ[C^{N3}(−2),H^{E1}(+1)]X and FdZ[C^{N3}(−2),H^{E1}(+1)]X for dZ and FdZ containing cross-linked oligos, respectively.

Representative drawings of compounds of the invention including cross-link 1 are:

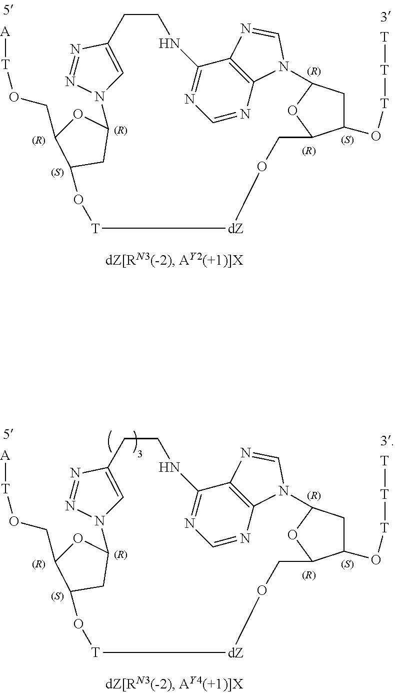

A representative drawing of a compound of the invention including cross-link 2 is:

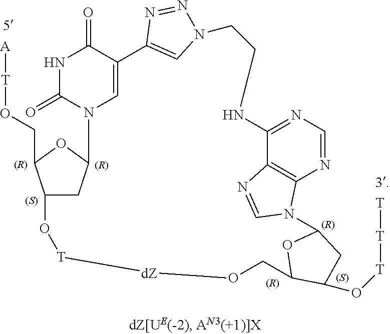

Representative drawings of compounds of the invention including cross-link 3 are:

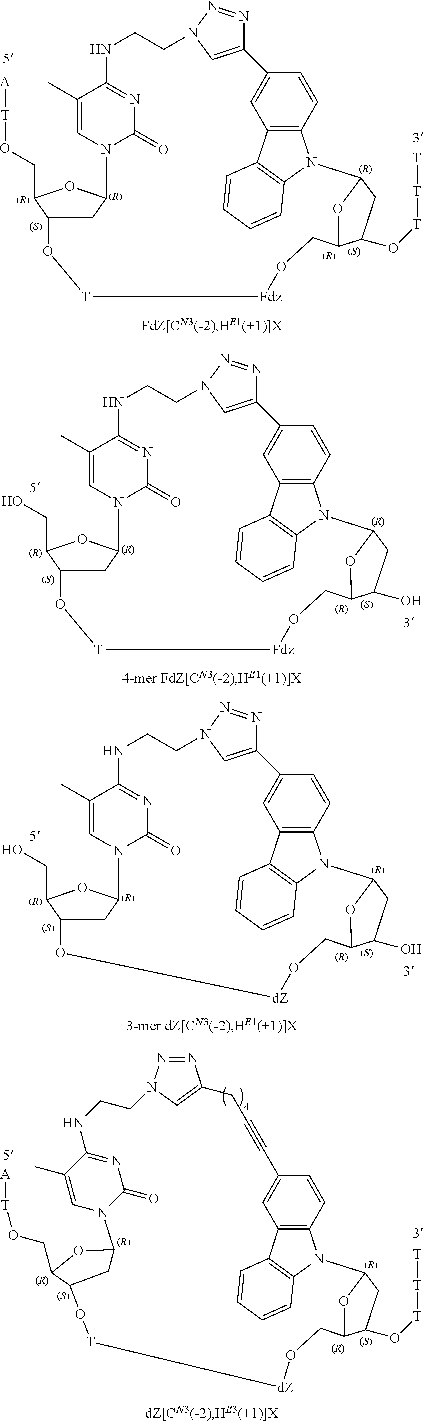

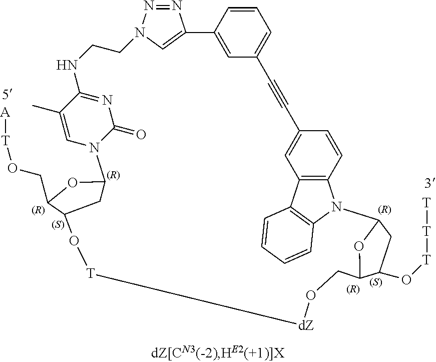

In another aspect the invention relates to a pharmaceutical composition comprising a compound of any one of formulae I to XIII and pharmaceutically acceptable carrier, diluent or excipient.

In one embodiment the pharmaceutical composition comprises an effective amount of the compound, preferably a therapeutically effective amount of the compound.

In one embodiment the pharmaceutical composition consists essentially of an effective amount of the compound, preferably a therapeutically effective amount of the compound.

In one embodiment the pharmaceutical composition is formulated for administration, or is in a form for administration, to a subject in need thereof.

In one embodiment administration is selected from the group consisting of is topical, intranasal, epidermal, transdermal, oral or parenteral.

In one embodiment administration is parenteral administration selected from the group consisting of direct application, systemic, subcutaneous, intraperitoneal or intramuscular injection, intravenous drip or infusion, inhalation, insufflation or intrathecal or intraventricular administration.

In one embodiment the pharmaceutical composition is formulated for, or is in a form for, parenteral administration in any appropriate solution, including sterile aqueous solutions which may also contain buffers, diluents and other suitable additives.

In one embodiment the pharmaceutical composition is formulated for or is in the form of an injection.

A person skilled in the art will be able to choose the appropriate mode of administration of a pharmaceutical composition as described herein with reference to the literature and as described herein. By way of non-limiting example, a systemic application would be preferred for the treatment and prevention of certain cancers whereas a local application would be preferred for the treatment of others, but not limited thereto.

Moreover, as will be understood by a person skilled in the art, a number of different types of pharmaceutical compositions can be prepared as described herein, and by following guidelines available to the skilled person in the art, for example, for systemic administration according to conventional formulation practice, see, e.g., “Remington's Pharmaceutical Sciences” and “Encyclopaedia of Pharmaceutical Technology”, but not limited thereto.

### 6. EXAMPLES

### 6.1. Materials and Methods

All reactions were performed in oven-dried glassware under an atmosphere of dry argon or nitrogen unless otherwise noted. Moisture-sensitive reactions were carried out using standard syringe septum techniques and under an inert atmosphere of argon or nitrogen. All solvents and reagents were purified by standard techniques unless otherwise noted. Solvents for filtration, transfers, and chromatography were certified ACS grade. Evaporation of solvents was carried out under reduced pressure on a rotary vacuum evaporator below 40° C. “Brine” refers to a saturated solution of sodium chloride in water. ¹H, ¹³C, ³¹P NMR spectra were recorded on Bruker 500- and 700-MHz spectrometers. Chemical shifts are reported in parts per million (ppm) downfield from tetramethylsilane. Spin multiplicities are described as s (singlet), bs (broad singlet), d (doublet), dd (double of doublets), dt (double of triplets), ddd (doublet of doublet of doublets), t (triplet), q (quartet), m (multiplet). Coupling constants are reported in Hertz (Hz). The assignments of signals were done using 2D homonuclear ¹H⁻¹H COSY, NOESY and heteronuclear ¹H⁻¹³C HMQC or HSQC, and HMBC spectra. NMR spectra were processed in TopSpin. High-resolution electrospray mass spectra were recorded on a Thermo Fisher Scientific Q Exactive Focus Hybrid Quadrupole-Orbitrap mass spectrometer. Ions generated by ESI were detected in positive ion mode for small molecules and negative ion mode for oligonucleotides. Total ion count (TIC) was recorded in centroid mode over the m/z range of 100-3,000 and analyzed using Thermo Fisher Xcalibur Qual Browser. Analytical thin layer chromatography (TLC) was performed on MERCK precoated silica gel 60-F254 (0.5-mm) aluminum plates. Visualization of the spots on TLC plates was achieved either by exposure to UV light or by dipping the plates into aqueous KMnO₄ and heating with a heat gun. Silica gel column chromatography was performed using silica gel 60 (40-63 μm). Oligonucleotide syntheses were carried out on a MerMade-4 DNA/RNA synthesizer (BioAutomation, USA) on a 5 μmol scale using standard manufacturer's protocol for unmodified nucleotides.

### 6.2. Example 1: Synthesis of DNA Precursors Containing Organic Azides and Alkynes for Internal Cross-Links 1-3

### 6.2.1. Synthesis of Azido Sugar Phosphoramidite and CPG Support

A method reported recently²⁰ described the synthesis of compound 2 from Hoffer's chlorosugar 1 (Scheme 1) using a Lewis acid and trimethylsilyl azide, but low β:α selectivity and moderate yields were reported. In contrast, the inventors employed a phase-transfer protocol²¹ with minor changes using NaN₃ and Bu₄NHSO₄ under vigorous stirring in saturated aqueous NaHCO₃ and chloroform followed by addition of Hoffer's chlorosugar 1. The reaction was finished in 20 min and after work-up resulted in almost pure azide 2 with high yield (88%) and β:α ratio of 16:1 (6% of α-anomer). Subsequent recrystallization from EtOH slightly increased the β:α ratio to 19:1 (5% of α-anomer). This protocol was optimized to a multi-gram scale synthesis of azide 2 in the inventors' laboratory. The controlled-pore glass (CPG) conjugated to azido-sugar 4 was prepared from a 5′-dimethoxytrityl (DMT) protected azido-sugar 3 by a slight modification of reported methods²²⁻²³ (Scheme 1). The loading on CPG was found to be 39 μmol/g by UV absorption of the DMT cation released upon treatment of a sample of the modified support 4 with a 3% solution of dichloroacetic acid in dichloromethane.

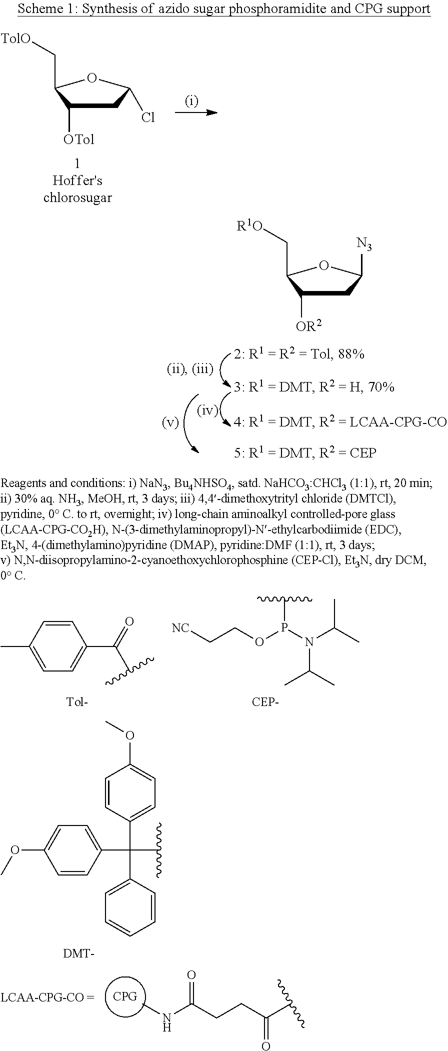

### 6.2.1.1. Synthesis of 2-deoxy-3,5-bis-O-(4-methylbenzoyl)-β-D-erythro-pentofuranosyl azide (2)

To a stirring solution of sodium azide (97.5 g, 1.5 mol) and tetrabutylammonium hydrogen sulfate (101.7 g, 0.3 mmol) in saturated sodium bicarbonate solution (1 L), chloroform (1 L) was added and the mixture was vigorously stirred for about 5 min until a milky emulsion was formed. Hoffer's chloro-sugar 1 (116.7 g, 0.3 mol) was rapidly added to the emulsion and the mixture was stirred 20 min. After the disappearance of starting material, the organic layer was washed with satd. sodium bicarbonate (1 L), water (2×1 L), dried over anhydrous sodium sulfate and was filtered. Solvent was evaporated in vacuo and the product was recrystallized from ethanol (450 mL) to yield compound 2 (105 g, 88%, β:α ratio of 19:1).

¹H NMR (500 MHz, DMSO-d₆): δ 7.94-7.84 (m, 4H, H-3); 7.36-7.29 (m, 4H, H-4); 5.87 (dd, 1H, J=6.0 Hz, J=4.2 Hz, H-1′); 5.56-5.50 (m, 1H, H-3′); 4.59-4.39 (m, 3H, H-4′,5′); 2.49-2.42 (m, 1H, H-2′a); 2.375, 2.370 (2s, 6H, H-6); 2.37-2.30 (m, 1H, H-2′b).

HRMS (ESI) m/z: [M+Na]⁺ Calcd for C₂₁H₂₁N₃O₅Na 418.1379. found 418.1372.

### 6.2.1.2. Synthesis of 2-deoxy-5-O-(4,4′-dimethoxytrityl)-β-D-erythro-pentofuranosyl azide (3)

Synthesis was performed by a similar procedure described earlier.²⁴ To a stirring solution of 2-deoxy-3,5-bis-O-(4-methylbenzoyl)-β-D-erythro-pentofuranosyl azide 2 (3.96 g, 10 mmol) in 370 mL methanol was added 37 mL of 30% aq. ammonia solution and kept it stirring for 3 days. After the disappearance of the starting material, volatiles were removed by rotary vacuum evaporator and residue was co-evaporated again with water to remove formed methyl toluate and then freeze dried from water to get the deprotected compound 2-deoxy-β-D-erythro-pentofuranosyl azide (1.65 g). This product was used without further purification to protect it with DMT. To a stirring solution of the deprotected azido sugar (1.65 g, 10 mmol) in dry pyridine (40 mL) at 0° C. 4,4′-dimethoxytrityl chloride (3.72 g, 11 mmol) was added and mixture was stirred at r.t. overnight. Pyridine was evaporated in vacuo. The residue was dissolved in 50 mL ethyl acetate and washed with brine (2×10 mL). The organic layer was dried over anhydrous sodium sulfate, filtered, and evaporated in vacuo. The crude product was purified by column chromatography over silica gel treated with 10% Et₃N in DCM and compound 3 was eluted with 5% EtOAc in DCM to afford the desired product 3 as a foam (3.13 g, 70%).

¹H NMR (500 MHz, DMSO-d₆): δ 7.47-7.42 (m, 2H, H-2); 7.34-7.26 (m, 6H, H-3, 2″); 7.25-7.19 (m, 1H, H-4); 6.92-6.86 (m, 4H, H-3″); 5.68 (dd, 1H, J=5.8 Hz, J=3.1 Hz, H-1′); 5.25 (d, 1H, J=5.0 Hz, OH); 4.20-4.12 (m, 1H, H-3′); 3.93-3.86 (m, 1H, H-4′); 3.73 (s, 6H, 2×CH₃); 3.15-3.02 (m, 2H, H-5′); 2.04-1.91 (m, 2H, H-2′).

HRMS (ESI) m/z: [M+Na]⁺ Calcd for C₂₆H₂₇N₃O₅Na 484.1848. found 484.1844.

### 6.2.1.3. Synthesis of modified azido-2-deoxyribose CPG (4)

Synthesis of the CPG support 4 was performed following the previously reported procedure.²² LCAA-CPG (3.0 g) with a pore size of 500 Å and 120-200 mesh size obtained commercially (ChemGenes Corporation) was activated by 3% trifluoroacetic acid in DCM (30 mL) and kept overnight with gentle stirring. The slurry was filtered and washed with 9:1 triethylamine:diisopropylamine (50 mL), DCM and diethylether, then dried in vacuo. Activated LCAA-CPG was then treated with succinic anhydride (6.6 mmol, 0.66 g) and 4-dimethylaminopyridine (DMAP) (0.8 mmol, 0.1 g) in anhydrous pyridine (12 mL) and stirred gently at r.t. overnight. The slurry was filtered off and washed successively with pyridine, DCM, and ether, then dried in vacuo. This carboxylic derivatized CPG was then coupled with compound 3 (0.4 mmol, 0.184 g) in presence of 1-ethyl-3-(3-dimethylaminopropyl) carbodiimide (EDC base) (2.7 mmol, 0.48 mL), triethylamine (20 μL) and DMAP (0.8 mmol, 0.1 g) in 1:1 pyridine:DMF (36 mL). After 72 hours pentafluorophenol (1.5 mmol, 0.27 g) was added and mixture was kept overnight. The slurry was filtered and washed with DCM and a mixture of 9:1 pyridine:piperidine (10 mL) during 5 min followed by DCM, acetonitrile, THF and again with DCM, then dried in vacuo. The CPG was then treated with the mixture of 2 mL of Cap A (acetic anhydride:2,6-lutidine:THF 1:1:8 v/v/v) and 2 mL of Cap B (1-methylimidazole:THF 4:21 v/v) for 2 hours, followed by washing with DCM, methanol, acetonitrile, THF and again with DCM and dried in vacuo. The load on the CPG 4 was determined to be 39 μmol/g based on UV absorption of DMT-cation at 504 nm (ε=76,000 L·mol⁻¹·cm⁻¹) that was released from an aliquot of compound by treatment with 3% dichloroacetic acid in DCM.

### 6.2.1.4. Synthesis of 3-O—(N,N-diisopropylamino-2-cyanoethoxyphosphanyl)-2-deoxy-5-O-(4,4′-dimethoxytrityl)-β-D-erythro-pentofuranosyl azide (5)

To a stirring solution of azido sugar 3 (0.46 g, 0.99 mmol) in dry DCM (5 mL), under argon at rt were added Et₃N (0.19 mL) followed by 2-cyanoethyl N,N-diisopropyl chlorophosphoramidite (0.25 mL, 1 mmol). After the consumption of the starting material, reaction mixture was washed with saturated NaHCO₃ solution (5×3 mL) followed by brine (5 mL). The organic layer was dried by passing through a column of anhydrous sodium sulfate. The concentration of azide phosphoramidite 5 in DCM solution (0.18 M) was calculated based on UV absorption of DMT-cation at 504 nm (ε=76,000 L/mol/cm) that was released from an aliquot of compound 5 by treatment with dichloroacetic acid in DCM.

¹H NMR (500 MHz, DMSO-d₆) δ 7.52-7.47 (m, 2H, H-2″); 7.41-7.37 (m, 3H, H-3″ and H-4″); 7.32-7.18 (m, 4H, H-2′″), 6.87-6.82 (m, 4H, H-3′″); 5.61 (dd, J=5.77 Hz, J=3.7 Hz, 1H, H-1′); 4.56-4.44 (m, 1H, H-3′); 4.22-4.15 (m, 2H, CH₂CH₂CN); 4.14-4.08 (m, 1H, H-4′); 3.79 (s), 3.78 (s) (6H, OCH₃); 3.60-3.50 (m, 2H, NCHCH₃); 3.32-3.21 (m, 2H, H-5′); 2.75-2.71 (m, 2H, 2.68-2.65 CH₂CH₂CN); 2.27-2.11 (m, 2H, H-2′a, 2′b); 1.18-1.13 (m, 9H), 1.04 (d, J=6.78 Hz) (12H, NCHCH₃).

³¹P NMR (202.5 MHz, DMSO-d₆, ref. 85% H₃PO₄) δ 148.97, 148.59 in ˜1:1 ratio.

### 6.2.2. Synthesis of modified 2′-deoxyadenosines

The second strategy relied on creation of internal cross-links 1-3 between azide- and alkyne-containing nucleotides (FIG. 2C-H). The synthesis of modified nucleosides is summarized in Table 1 and Schemes 2-4.

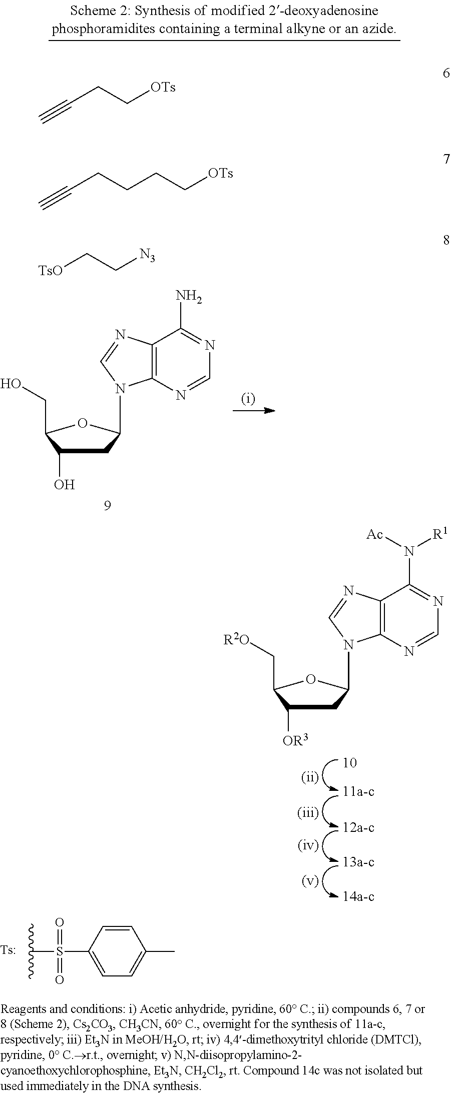

TABLE 1
Yield of modified 2′-deoxyadenosine phosphoramidites
(Scheme 2) containing a terminal alkyne or an azide.

| # | R¹ | R² | R³ | Yield, % |
|---|---|---|---|---|
| 10 | H | Ac | Ac | 75 |
| 11a | (CH₂)₂C≡CH | Ac | Ac | 80 |
| 11b | (CH₂)₄C≡CH | Ac | Ac | 85 |
| 11c | (CH₂)₂N₃ | Ac | Ac | 80 |
| 12a | (CH₂)₂C≡CH | H | H | 82 |
| 12b | (CH₂)₄C≡CH | H | H | 82 |
| 12c | (CH₂)₂N₃ | H | H | 80 |
| 13a | (CH₂)₂C≡CH | DMT | H | 89 |
| 13b | (CH₂)₄C≡CH | DMT | H | 86 |
| 13c | (CH₂)₂N₃ | DMT | H | 89 |
| 14a | (CH₂)₂C≡CH | DMT | P(NiPr)₂OCH₂CH₂CN | 92 |
| 14b | (CH₂)₄C≡CH | DMT | P(NiPr)₂OCH₂CH₂CN | 90 |
| 14c | (CH₂)₂N₃ | DMT | P(NiPr)₂OCH₂CH₂CN | Not isolated but used |
| | | | | immediately in DNA synthesis |

To synthesize 2′-deoxyadenosine phosphoramidites the inventors introduced azido- and alkyne-containing alkane linkers into the N⁶ position of dA. In the past various methods employed for N⁶ alkylation of dA usually relied upon alkylation in the N² position followed by subsequent Dimroth rearrangement to the N⁶-alkylated product.²⁵⁻²⁶ In contrast to previous reports, we observed a direct alkylation of the N⁶ position in triacetylated dA (7) using tosylated alkyne and azide-containing alcohols (6-8)²⁷ and Cs₂CO₃ as the base. The alkylation at the N⁶ position of dA was confirmed for compounds 11a-c by ¹H,¹³C heteronuclear multiple bond correlation (HMBC) NMR experiment in which three-bond correlations are detected. For example, in the HMBC spectrum of compound 11c, we observed that proton signals of the CH₂-group of the azidoalkyl linker at 4.31 ppm had a cross-peak not only with C⁶ of adenine at 152.58 ppm but also with the carbonyl carbon of the acetyl group at 170.06 ppm.

This is only possible to observe for N⁶ and not for N¹ alkylated dA (three versus five bonds between hydrogen of CH₂ and carbon of C═O, respectively). Similar patterns of cross-peaks were observed for compounds 11a,b. After the selective deprotection of 3′- and 5′-acetyl groups, the 5′-hydroxy group of compounds 12a-c was converted into the 4,4′-dimethoxytrityl derivatives 13a-c under standard conditions. Phosphoramidites 14a-b for alkyne-containing dA were obtained using standard phosphitylation reaction with N,N-diisopropylamino-2-cyanoethoxychlorophosphine and Et₃N in dry CH₂Cl₂ followed by aqueous work-up and silica gel purification. However, this protocol has to be modified for azide-containing dA.

A pilot phosphitylation reaction was performed on compound 13c using previously described conditions²⁸ in dry CDCl₃ producing the phosphoramidite 14c with a purity of more than 70% as shown by ¹H and ³¹P NMR spectra. This suggests that compound 14c can be used for automated DNA synthesis, with a limitation that the freshly prepared phosphoramidite should be used immediately after its synthesis because the storage of this solution at room temperature for 24 hours results in significant degradation of compound 14c as evidenced by NMR. For automated DNA synthesis, the reaction was performed under the same conditions using dry dichloromethane as a solvent to obtain phosphoramidite 14c. A similar protocol was used for preparation of phosphoramidite of dR^{N3} (compound 5, Scheme 1).

### 6.2.2.1. Synthesis of but-3-yn-1-yl 4-methylbenzenesulfonate (6)

But-3-yn-1-ol (2.0 g, 20.37 mmol) in DCM (50 mL) was cooled to 0° C. and then DMAP (34 mg, 0.27 mmol), tosyl chloride (6.52 g, 34.19 mmol) and Et₃N (4.8 mL, 34.19 mmol) were added. The reaction mixture was stirred for 30 min and then diluted with 50 mL more of DCM and washed with brine (50×2 mL). Organic layer was dried over anhydrous sodium sulfate, filtered, concentrated in vacuo. The crude product was purified by column chromatography over silica gel (60-120 mesh) and eluting with hexane/EtOAc (7:3) to afford the desired product 6 (5.90 g, 92%).

¹H NMR (500 MHz, CDCl₃): δ 7.83 (d, J=8.37 Hz, 2H, H2, H6); 7.38 (d, J=8.44 Hz, 2H, H3, H5); 4.13 (t, J=7.04 Hz, 2H, OCH₂CH₂CCH); 2.58 (ddd, J=7.07, 7.03, 2.74 Hz, 2H, OCH₂CH₂CCH); 2.47 (s, 3H, ArCH₃); 1.99 (t, J=2.67 Hz, 1H, OCH₂CH₂CCH).

HRMS (ESI) m/z: [M+Na]⁺ Calcd for C₁₁H₁₂NaO₃S: 247.0399. Found: 247.0396.

### 6.2.2.2. Synthesis of hex-5-yn-1-yl 4-methylbenzenesulfonate (7)

Compound 7 was synthesized using the same protocol as for compound 6 with 98% yield.

¹H NMR (500 MHz, CDCl₃): δ 7.82 (d, J=8.36 Hz, 2H, H2, H6); 7.37 (d, J=8.34 Hz, 2H, H3, H5); 4.08 (t, J=7.04 Hz, 2H, OCH₂CH₂CH₂CH₂CCH); 2.48 (s, 3H, ArCH₃); 2.19 (ddd, J=6.97 Hz, 6.90 Hz, 2.59 Hz, 2H, CH₂CH₂CH₂CH₂CCH); 1.95 (t, J=2.63 Hz, 1H, OCH₂CH₂CH₂CH₂CCH); 1.83-1.78 (m, 2H, OCH₂CH₂CH₂CH₂CCH); 1.61-1.55 (m, 2H, OCH₂CH₂CH₂CH₂CCH).

HRMS (ESI) m/z: [M+Na]⁺ Calcd for C₁₃H₁₆O₃S 275.0712. Found 275.0707.

### 6.2.2.3. Synthesis of 2-azidoethyl-4-methylbenzenesulfonate (8)

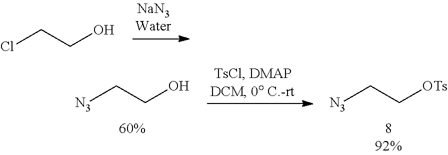

2-Azidoethan-1-ol was prepared according to a reported procedure²⁹ and converted to compound 8 using the same protocol as for compound 6. Yield 92%.

¹H NMR (500 MHz, DMSO-d₆): δ 7.79 (d, J=8.3 Hz, 2H); 7.34 (d, J=8.1 Hz, 2H,); 4.16 (t, 2H, J=5.2 Hz, CH₂CH₂N₃); 3.48 (t, 2H, J=5.2 Hz, CH₂CH₂N₃); 2.45 (s, 3H, Ar—CH₃).

HRMS (ESI) m/z: [M+Na]⁺ Calcd for C₉H₁₁N₃O₃SNa 264.0419. found 264.0413.

### 6.2.2.4. Synthesis of N⁶,3′,5′-Triacetyl-2′-deoxyadenosine (10)

N⁶,3′,5′-Triacetyl-2′-deoxyadenosine 10 was prepared similar to a reported procedure with some modifications.²⁶ A mixture of 2′-deoxyadenosine 9 (1.0 g, 3.98 mmol), pyridine (7.5 mL) and Ac₂O (3.5 mL) was stirred at r.t. overnight. The resulting solution was heated to 60° C. for 6 h. After the disappearance of the starting material, monitored by TLC (CH₂C₂—MeOH, 95:5), the reaction was cooled down and quenched with excess of EtOH (20 mL). Volatiles were evaporated in vacuo. Traces of pyridine were co-evaporated with successive portions of EtOH and MeOH (20 mL each). The resultant oily liquid was diluted with EtOAc (100 mL) and washed with brine (2×20 mL). The organic layer was dried over anhydrous sodium sulfate, filtered, and evaporated in vacuo. The crude product was purified by column chromatography on silica gel, eluting with CH₂Cl₂/MeOH (9.5:0.5) to afford the desired product 10 as a white solid (1.15 g, 76%).

¹H NMR (500 MHz, DMSO-d₆): δ 10.70 (s, 1H, NH); 8.663, 8.660 (2s, 2H, H-2, H-8); 6.48 (dd, 1H, J_{1′,2′}=6.3, 7.8 Hz, H-1′); 5.44 (dt, 1H, J=2.8, 6.5 Hz, H-3′); 4.32 (dd, 1H, J_{4′,5′a}=4.2 Hz, J_{5′a,5′b}=11.0 Hz, H-5′a); 4.28 (ddd, 1H, J_{3′,4′}=2.7 Hz, J_{4′,5′a}=4.2 Hz, J_{4′,5′b}=5.5 Hz, H-4′); 4.22 (dd, 1H, J_{4′,5′b}=5.5 Hz, J_{5′a,5′b}=11.0 Hz, H-5′b); 3.20 (ddd, 1H, J_{2′a,3′}=2.8 Hz, J_{2′a,1′}=6.3 Hz, J_{2′a,2′b}=14.2 Hz, H-2′a); 2.60 (ddd, 1H, J_{2′b,3′}=6.6 Hz, J_{2′b,1′}=7.8 Hz, J_{2′a,2′b}=14.2 Hz, H-2′b); 2.26 (s, 3H, NHCOCH₃); 2.10, 2.00 (2s, 6H, OCOCH₃).

HRMS (ESI) m/z: [M+H]⁺ Calcd for C₁₆H₂₀N₅O₆ 378.1414. found 378.1397.

### 6.2.2.5. General Protocol for alkylation of N⁶,3′,5′-triacetyl-2′-deoxyadenosine (10)

To a stirring solution of N⁶,3′,5′-triacetyl-2′-deoxyadenosine (10, 1.0 g, 2.65 mmol) in acetonitrile (15 mL), Cs₂CO₃ (2.50 g, 7.67 mmol) and one of tosylated alcohols 6-8 (15.80 mmol) were added and mixture was heated to 60° C. After the disappearance of the starting material on TLC the reaction mixture was diluted with EtOAc (50 mL) and washed with water (2×10 mL). Organic layer was dried over anhydrous sodium sulfate, filtered, and concentrated in vacuo. The crude product was purified by silica gel column chromatography using CH₂Cl₂/MeOH (9:1) to get desired compound as a sticky solid.

### 6.2.2.5.1. Characterization of N⁶-(but-3-yn-1-yl)-N⁶,3′,5′-triacetyl-2′-deoxyadenosine (1a)

Yield: 0.64 g, 80%.

¹H NMR (500 MHz, DMSO-d₆): δ 8.83 (s, 1H, H-8); 8.82 (s, 1H, H-2); 6.53 (dd, 1H, J_{1′,2′,a}=6.8, J_{1′,2′b}=7.0 Hz, H-1′); 5.46 (dd, J_{3′,2′b}=3.28 Hz, J_{3′,4′}=6.07 Hz, 1H, H-3′); 4.35-4.31 (m, 2H, H-5′); 4.27-4.24 (m, 1H, H-4′); 4.21 (t, J=7.45 Hz, 2H, NCH₂CH₂CCH); 3.20 (ddd, 1H, J_{2′a,3′}=2.8 Hz, J_{2′a,1′}=6.8 Hz, J_{2′a,2′b}=13.3 Hz, H-2′a); 2.63 (ddd, 1H, J_{2′b,3′}=3.3 Hz, J_{2′b,1′}=6.3 Hz, J_{2′a,2′b}=13.3 Hz, H-2′b); 2.50 (app t, NCH₂CH₂CCH); 2.19 (s, 3H, NCOCH₃); 2.12 (s, 3H, OCOCH₃); 2.01 (s, 3H, OCOCH₃).

HRMS (ESI) m/z: [M+H]⁺ Calcd for C₂₀H₂₄N₅O₆ 430.1721. Found 430.1713.

### 6.2.2.5.2. Characterization of N⁶-(hex-5-yn-1-yl)-N⁶,3′,5′-triacetyl-2′-deoxyadenosine (11b)

Yield: 1.03 g, 85%.

¹H NMR (500 MHz, DMSO-d₆): δ 8.86 (s, 1H, H-8); 8.83 (s, 1H, H-2); 6.53 (dd, 1H, J_{1′,2′,a}=6.9, J_{1′,2′b}=7.1 Hz, H-1′); 5.46 (dd, J_{3′,2′b}=6.07 Hz, J_{3′,4′}=3.28 Hz, 1H, H-3′); 4.35-4.30 (m, 1H, H-5′a); 4.28-4.24 (m, 1H, H-4′); 4.23 (dd, J_{5′b,5′a}=11.23 Hz, J_{5′b,4′}=5.50 Hz, 1H, H-5′b); 4.11 (t, J=7.45 Hz, 2H, NCH₂CH₂CH₂CH₂CCH); 3.21 (dd, J_{2′a,2′b}=2.8 Hz, J_{2′a,1′}=6.9 Hz, 1H, H-2′a); 2.64 (dd, 1H, J_{2′b,2′a}=6.34 Hz, J_{2′b, 3′}=2.79 Hz, 1H, H-2′b); 2.12 (s, 3H, OCOCH₃); 2.10-2.09 (m, 2H, NCH₂CH₂CH₂CH₂CCH); 2.08 (s, 3H, OCOCH₃); 1.92 (s, 3H, NCOCH₃) 1.54 (q, J=7.31 Hz, 2H, NCH₂CH₂CH₂CH₂CCH); 1.38 (q, J=7.84 Hz, 2H, NCH₂CH₂CH₂CH₂CCH).

HRMS (ESI) m/z: [M+Na]⁺ Calcd for C₂₂H₂₇N₅O₆Na 480.1859. Found 480.1853.

### 6.2.2.5.3. Characterization of N⁶-(2-azidoethyl)-N⁶,3′,5′-triacetyl-2′-deoxyadenosine (11c)

Yield: 0.94 g, 80%.

¹H NMR (500 MHz, DMSO-d₆): δ 8.84 (s, 1H, H-8); 8.82 (s, 1H, H-2); 6.52 (dd, 1H, J_{1′,2a′}=6.7, J_{1′,2′b} 7.2 Hz, H-1′); 5.45 (dt, 1H, J=2.3, 6.4 Hz, H-3′); 4.32 (dd, 1H, J_{4′,5′a}=4.2 Hz, J_{5′a,5′b}=10.6 Hz, H-5′a); 4.31-4.28 (m, 3H, H₅′b and NCH₂CH₂N₃); 4.23 (ddd, 1H, J_{3′,4′}=2.3 Hz, J_{4′,5′a}=4.4 Hz, J_{4′,5′b}=5.9 Hz, H-4′); 3.52 (t, J=7.0 Hz, 2H, NCH₂CH₂N₃); 3.20 (ddd, 1H, J_{2′a,3′}=2.3 Hz, J_{2′a,1′}=6.7 Hz, J_{2′a,2′b}=14.3 Hz, H-2′a); 2.63 (ddd, 1H, J_{2′b,3′}=6.4 Hz, J_{2′b,1′}=7.2 Hz, J_{2′a,2′b}=14.3 Hz, H-2′b); 2.17 (s, 3H, NCOCH₃); 2.10 (s, 3H, OCOCH₃); 1.99 (s, 3H, OCOCH₃).

IR ATR (cm⁻¹): 3440.67, 2306.61, 2339.80, 2251.73, 2124.62, 1667.50, 1058.50, 1037.65, 1008.07, 823.02, 760.59, 667.93, 624.85.

HRMS (ESI) m/z: [M+H]⁺ Calcd for C₁₈H₂₃N₈O₆ 447.1741. found 447.1737.

### 6.2.2.6. General Procedure for Removal of 3′,5′-O-Acetyl Protecting Groups

To a stirring solution of one of the compounds 11a-c (2.0 mmol) in MeOH:water (10:10 mL) Et₃N (1.5 mL) was added at r.t. The reaction was monitored by TLC. After 15 min volatiles were evaporated in vacuo. The crude product was either triturated with ether providing the desired white solid for 12a-b (85% and 82% yields) or purified by silica gel column chromatography using CH₂Cl₂/MeOH (7:3) to get desired compound 12c as a semisolid (89% yield).

### 6.2.2.6.1. Characterization of N⁶-(but-3-yn-1-yl)-N⁶-acetyl-2′-deoxyadenosine (12a)

¹H NMR (500 MHz, DMSO-d₆): δ 8.83 (s, 1H, H-8); 8.81 (s, 1H, H-2); 6.48 (app t, J=6.72 Hz, 1H, H-1′); 4.46-4.42 (m, 1H, H-3′); 4.18 (t, J=7.40 Hz, 2H, NCH₂CH₂CCH); 3.91-3.89 (m, 1H, H-4′); 3.63 (dd, J_{5′a,5′b}=11.74 Hz, J_{5′a,4′}=4.69 Hz, 1H, H-5′a) 3.54 (dd, 1H, J_{5′b,5′a}=11.74 Hz, J_{5′b,4′}=4.76 Hz, 1H, H-5′b); 2.74 (ddd, J_{2′a,2′b}=11.23 Hz, J_{2′a,3′}=7.00 Hz, J_{2′a,1′}=5.89 Hz, 1H, H-2′a); 2.69 (t, J=2.63 Hz, 1H, NHCH₂CH₂CCH); 2.49-2.45 (m, 2H, NHCH₂CH₂CCH); 2.39-2.34 (m, 1H, H-2′b); 2.18 (s, 3H, NCOCH₃).

HRMS (ESI) m/z: [M+H]⁺ Calcd for C₁₆H₂₀N₅O₄ 346.1510. Found 346.1505.

### 6.2.2.6.2. Characterization of N⁶-(hex-5-yn-1-yl)-N⁶-acetyl-2′-deoxyadenosine (12b)

¹H NMR (500 MHz, DMSO-d₆): δ 8.84 (s, 1H, H-8); 8.83 (s, 1H, H-2); 6.49 (app t, J=6.74 Hz, 1H, H-1′); 4.47-4.46 (m, 1H, H-3′); 4.10 (t, J=7.22 Hz, 2H, NCH₂CH₂CH₂CH₂CCH); 3.92 (q, J=4.38, 1H, H-4′); 3.65 (dd, J_{5′a,5′b}=11.82 Hz, J_{5′a,4′}=4.60 Hz, 1H, H-5′a) 3.56 (dd, 1H, J_{5′b,5′a}=11.82 Hz, J_{5′b,4′}=4.60 Hz, 1H, H-5′b); 2.79 (ddd, J_{2′a,2′b}=13.83 Hz, J_{2′a,3′}=6.71 Hz, J_{2′a,1′}=6.49 Hz, 1H, H-2′a); 2.65 (t, J=2.70 Hz, NCH₂CH₂CH₂CH₂CCH); 2.51-2.38 (m, 1H, H-2′b); 2.10 (s, 1H, NHCOCH₃); 2.09-2.07 (m, 2H, NCH₂CH₂CH₂CH₂CCH); 1.56-1.50 (m, 2H, NCH₂CH₂CH₂CH₂CCH); 1.41-1.35 (m, 2H, NCH₂CH₂CH₂CH₂CCH).

HRMS (ESI) m/z: [M+H]⁺ Calcd for C₁₈H₂₄N₅O₄ 374.1823. Found 374.1817.

### 6.2.2.6.3. Characterization of N⁶-(2-azidoethyl)-N⁶-acetyl-2′-deoxyadenosine (12c)

¹H NMR (500 MHz, DMSO-d₆): δ 8.83 (s, 1H, H-8); 8.82 (s, 1H, H-2); 6.49 (app t, J=6.7 Hz, 1H, H-1′); 5.43 (s, 1H, 3′-OH), 5.05 (s, 1H, 5′-OH), 4.45 (dt, 1H, J=3.2, 5.6 Hz, H-3′); 4.29 (t, J=6.1 Hz, 2H, NCH₂CH₂N₃); 3.90 (ddd, 1H, J₃,4′=2.3 Hz, J_{4′,5′b}=4.6 Hz, J_{4′,5′a}=5.9 Hz, H-4′); 3.63 (dd, 1H, J_{4′,5′a}=5.9 Hz, J_{5′a,5′b}=11.7 Hz, H-5′a); 3.54 (dd, 1H, J_{4′,5′b}=4.6 Hz, J_{5′a,5′b}=11.7 Hz, H-5′b); 3.52 (t, 2H, J=6.1 Hz, 2H, NCH₂CH₂N₃); 2.77 (ddd, 1H, J_{2′a,3′}=2.8 Hz, J_{2′a,1′}=6.3 Hz, J_{2′a,2′b}=13.2 Hz, H-2′a); 2.37 (ddd, 1H, J_{2′b,3′}=6.2 Hz, J_{2′b,1′}=9.8 Hz, J_{2′a,2′b}=13.2 Hz, H-2′b); 2.16 (s, 3H, NCOCH₃).

HRMS (ESI) m/z: [M+H]⁺ Calcd for C₁₄H₁₈N₈O₄Na 385.1349. found 385.1342.

### 6.2.2.7. General Protocol for 5′-O-4,4′-dimethoxytritylation of nucleosides

To a stirring solution of a nucleoside (1.8 mmol) in dry pyridine (5 mL), 4,4′-dimethoxytrityl chloride (0.67 g, 2.0 mmol) was added at 0° C. and the mixture was stirred at r.t. overnight under argon. After consumption of the starting nucleoside (TLC analysis), H₂O (1 mL) was added to quench the reaction. Solvents were evaporated in vacuo and the residue was dissolved in 50 mL DCM, washed with brine (2×10 mL), organic layer was dried over anhydrous sodium sulfate, filtered, and concentrated in vacuo. The crude product was purified by column chromatography over silica gel treated with 10% Et₃N in CH₂Cl₂ and eluted with 5% MeOH in CH₂Cl₂ to afford the desired product as a foam.

### 6.2.2.7.1. Characterization of 5′-O-(4,4′-dimethoxytrityl)-N⁶-(but-3-yn-1-yl)-N⁶-acetyl-2′-deoxyadenosine (13a)

Yield: 0.68 g, 90%.

¹H NMR (500 MHz, DMSO-d₆): δ 8.73 (s, 1H, H-8); 8.71 (s, 1H, H-2); 7.31-7.30 (m, 2H,H-2″); 7.21-7.17 (m, 7H, H-3″, 4″ and 2′″); 6.81-6.76 (m, 4H, H-3′″); 6.51 (t, J=6.33 Hz, 1H, H-1′); 5.42 (d, 1H, J_{3′,OH}=4.63 Hz, H—OH) 4.51-4.49 (m, 1H, H-3′); 4.17 (t, J=7.70 Hz, 2H, NCH₂CH₂CH); 4.04-4.01 (m, 1H, H-4′); 3.70 (s, 3H, OCH₃); 3.71 (s, 3H, OCH₃); 3.23-3.16 (m, 2H, H-5′); 2.94-2.88 (m, 1H, 1H, H-2′a); 2.69 (t, J=2.57 Hz, 1H, NCH₂CH₂CCH); 2.47-2.45 (m, 2H, NCH₂CH₂CCH); 2.41-2.39 (m, 1H, H-2′b); 2.12 (s, 3H, NCOCH₃).

HRMS (ESI) m/z: [M+H]⁺ Calcd for C₃₇H₃₈N₅O₆ 648.2817. Found 648.2805.

### 6.2.2.7.2. Characterization of 5′-O-(4,4′-dimethoxytrityl)-N⁶-(hex-5-yn-1-yl)-N⁶-acetyl-2′-deoxyadenosine (13b)

Yield: 0.93 g, 86%.

¹H NMR (500 MHz, DMSO-d₆): δ 8.77 (s, 1H, H-8); 8.73 (s, 1H, H-2); 7.32-7.29 (m, 2H, H-2″); 7.21-7.17 (m, 7H, H-3″, 4″ and 2′″); 6.81-6.78 (m, 4H, H-3′″); 6.51 (t, J=6.56 Hz, 1H, H-1′); 5.44 (d, 1H, J_{3′,OH}=4.63 Hz, H—OH) 4.55-4.53 (m, 1H, H-3′); 4.08 (t, J=7.21 Hz, 2H, NCH₂CH₂CH₂CH₂CCH); 4.04-4.01 (m, 1H, H-4′); 3.72 (s, 6H, OCH₃); 3.20 (d, J=4.98 Hz, 2H, H-5′); 2.98-2.93 (m, 1H, 1H, H-2′a); 2.64 (t, J=2.66 Hz, 1H, NCH₂CH₂CH₂CH₂CCH); 2.45-2.40 (m, 1H, H-2′b); 2.01 (s, 3H, NCOCH₃); 2.04-2.00 (m, 2H, NCH₂CH₂CH₂CH₂CCH); 1.51-1.47 (m, 2H, NCH₂CH₂CH₂CH₂CCH); 1.37-1.31 (m, 2H, NCH₂CH₂CH₂CH₂CCH).

HRMS (ESI) m/z: [M+H]⁺ Calcd for C₃₉H₄₂N₅O₆ 676.3130. Found 676.3105.

### 6.2.2.7.3. Characterization of 5′-O-(4,4′-dimethoxytrityl)-N⁶-(2-azidoethyl)-N⁶-acetyl-2′-deoxyadenosine (13c)

Yield: 0.81 g, 74%.

¹H NMR (500 MHz, DMSO-d₆): δ 8.75 (s, 1H, H-8); 8.72 (s, 1H, H-2); 7.32-7.30 (m, 2H,H-2″); 7.21-7.16 (m, 7H, H-3″, 4″ and 2′″); 6.81-6.77 (m, 4H, H-3′″); 6.51 (dd, 1H, J_{1′,2′}=5.9, 6.7 Hz, H-1′); 5.43 (d, 1H, J_{3′OH}=4.65 Hz, 3′-OH); 4.54-4.50 (m, 1H, H-3′); 4.27 (t, J=6.17 Hz, 2H, NCH₂CH₂N₃); 4.09-4.02 (m, 1H, H-4′); 3.714 (s, 3H, OCH₃); 3.710 (s, 3H, OCH₃); 3.49 (t, J=6.10 Hz, 1H, NCH₂CH₂N₃); 3.23-3.16 (m, 2H, H-5′); 2.95-2.90 (m, 1H, 1H, H-2′a); 2.43-2.38 (m, 1H, H-2′b); 2.11 (s, 3H, NCOCH₃).

HRMS (ESI) m/z: [M+H]⁺ Calcd for C₃₅H₃₇N₈O₆ 665.2836. found 665.2831.

### 6.2.2.8. General Procedure for Preparation of Nucleoside Phosphoramidites

To a stirring solution of 5′-O-DMT protected nucleoside (0.66 mmol) in dry DCM (10 mL) under argon at rt were added Et₃N (0.12 mL, 0.86 mmol) followed by 2-cyanoethyl N,N-diisopropyl chlorophosphoramidite (0.17 g, 0.71 mmol). After the disappearance of the starting material in 10 min the reaction mixture was washed with saturated sodium bicarbonate solution (5×3 mL) followed by brine (5 mL). The organic layer was dried by passing through anhydrous sodium sulfate column and the crude product was purified by column chromatography over silica gel (60-120 mesh) saturated with Et₃N (10%) and eluting with CH₂Cl₂/acetone (9:1) to give a white foam.

### 6.2.2.8.1. Characterization of 5′-O-(4,4′-dimethoxytrityl)-N⁶-(but-3-yn-1-yl)-N⁶-acetyl-3′-O—(N,N-diisopropylamino-2-cyanoethoxyphosphanyl)-2′-deoxyadenosine (14a)

Yield: 0.45 g, 85%.

¹H NMR (500 MHz, DMSO-d₆): δ 8.74, 8.73 (2s, 1H, H-8); 8.70, 8.69 (2s, 1H, H-2); 7.32-7.29 (m, 2H, H-2″); 7.21-7.16 (m, 7H, H-3″, H-4″ and H-2′″); 6.81-6.76 (m, 4H, H-3′″); 6.51 (q, J=7.24 Hz, 6.69 Hz, 1H, H-1′); 4.85-4.79 (m, 1H, H-3′); 4.19-4.16 (m, 2H, NCH₂CH₂CH), 4.15-4.13 (m, 1H, H-4′); 3.81-3.57 (m, 2H, CH₂CH₂CN); 3.71 (s), 3.70 (s) (6H, OCH₃); 3.68-3.63 (m, 1H, NCH₂CH₂CN); 3.60-3.50 (m, 2H, NCHCH₃); 3.30-3.20 (m, 2H, H-5′); 3.14-3.07 (m, 1H, H-2′b); 2.76 (t, 1H, J=5.83 Hz, 1H, NCH₂CH₂CCH); 2.68-2.65 (m, 2H, CH₂CH₂CN); 2.63-2.51 (m, 1H, H-2′a); 2.48-2.46 (m, 2H, NCH₂CH₂CCH); 2.11, 2.12 (2s, 3H, NCOCH₃); 1.14, 1.12 (2d, J=6.8 Hz); 1.04 (d, J=6.78 Hz) (12H, NCHCH₃).

³¹P NMR (202.5 MHz, DMSO-d₆, ref. 85% H₃PO₄): δ 147.83, 147.20 in −1:1 ratio.

HRMS (ESI) m/z: [M+H]⁺ Calcd for C₄₆H₅₅N₇₀₇P 848.3895. Found 848.3870.

### 6.2.2.8.2. Characterization of 5′-O-(4,4′-dimethoxytrityl)-N⁶-(hex-5-yn-1-yl)-N⁶-acetyl-3′-O—(N,N-diisopropylamino-2-cyanoethoxyphosphanyl)-2′-deoxyadenosine (14b)

Yield: 0.7 g, 90%.

¹H NMR (500 MHz, DMSO-d₆): δ 8.76, 8.75 (2s, 1H, H-8); 8.74, 8.73 (2s, 1H, H-2); 7.30-7.29 (m, 2H, H-2″); 7.21-7.17 (m, 7H, H-3″, H-4″ and H-2′″); 6.82-6.77 (m, 4H, H-3′″); 6.53 (q, J=7.17 Hz, 1H, H-1′); 4.90-4.83 (m, 1H, H-3′); 4.19, 4.13 (2q, J=4.68 Hz, 1H, H-4′); 4.08 (t, J=6.83 Hz, 2H, NCH₂CH₂CH₂CH₂CCH); 3.72 (s), 3.71 (s) (6H, OCH₃); 3.69-3.63 (m, 2H, OCH₂CH₂CN); 3.58-3.50 (m, 2H, NCHCH₃); 3.22-3.12 (m, 2H, H-5′); 2.76 (t, 1H, J=5.83 Hz, 1H, NCH₂CH₂CH₂CH₂CCH); 2.69-2.64 (m, 2H, OCH₂CH₂CN); 2.63-2.51 (m, 1H, H-2′a); 2.05, 2.04 (2s, 3H, NCOCH₃); 2.03-2.01 (m, 2H, NCH₂CH₂CH₂CH₂CCH); 1.50-1.46 (m, 2H, NCH₂CH₂CH₂CH₂CCH); 1.36-1.30 (m, 2H, NCH₂CH₂CH₂CH₂CCH); 1.15 (d, J=6.8 Hz), 1.05 (d, J=6.78 Hz) (12H, NCHCH₃).

³¹P NMR (202.5 MHz, DMSO-d₆, ref. 85% H₃PO₄): δ 147.86, 147.25 in −1:1 ratio.

HRMS (ESI) m/z: [M+H]⁺ Calcd for C₄₈H₅₉N₇₀₇P 686.4208. Found 686.4181.

### 6.2.2.9. Synthesis of 5′-O-(4,4′-dimethoxytrityl)-N⁶-(2-azidoethyl)-N⁶-acetyl-3′-O—(N,N-diisopropylamino-2-cyanoethoxyphosphanyl)-2′-deoxyadenosine (14c)

To a stirring solution of compound 13c (0.6 g, 0.90 mmol) in dry CDCl₃(5 mL), Et₃N (0.16 mL, 1 mmol) followed by 2-cyanoethyl-N,N-diisopropyl chlorophosphoramidite (0.27 mL, 1.08 mmol) were added under argon at 0° C. After the consumption of the starting material, reaction mixture was washed with saturated NaHCO₃ solution (2×5 mL) followed by brine (5 mL). The organic layer was dried by passing through a column (12×1.5 cm) of anhydrous sodium sulfate. The solution of compound 14c in CDCl₃ contains excess of phosphitylation reagent as shown by NMR. For automated DNA synthesis, the reaction was performed under the same conditions using dry DCM as a solvent. The concentration of azide phosphoramidite in DCM solution was calculated as 0.2 M based on UV absorption of DMT-cation at 504 nm (ε=76,000 L·mol⁻¹·cm⁻¹) that was released from an aliquot of compound 14c by treatment with 3% dichloroacetic acid in DCM.

¹H NMR (500 MHz, CDCl₃): δ 8.72 (s, 1H, H-8); 8.27, 8.24 (2s, 1H, H-2); 7.39-7.37 (m, 2H, H-2″); 7.29-7.19 (m, 7H, H-3″, H-4″ and H-2′″); 6.80-6.77 (m, 4H, H-3′″); 6.54-6.51 (m, 1H, H-1′); 4.84-4.77 (m, 1H, H-3′); 4.40-4.37 (m, 2H, NCH₂CH₂N₃); 4.22-4.16 (m, 1H, H-4′); 4.15-4.09 (m, 2H, CH₂CH₂CN); 3.77 (2s, 6H, OCH₃); 3.63-3.55 (m, 2H, NCH₂CH₂CN); 3.60-3.50 (m, 2H, NCHCH₃); 3.50-3.46 (m, 2H, H-5′); 2.93 (ddd, 1H, J=6.6, 9.8, 13.4 Hz, H-2′b); 2.76-2.73 (m, 2H, CH₂CH₂CN); 2.64-2.61 (m, 1H, H-2′a); 2.58-2.54 (m, 2H, NCH₂CHN₃); 2.25, 2.24 (2s, 3H, NCOCH₃); 1.20-1.18 (m, 10H, NCHCH₃); 1.13 (d, 2H, NCHCH₃, J=6.5 Hz).

³¹P NMR (202.5 MHz, DMSO-d₆, ref. 85% H₃PO₄): δ 148.8.

### 6.2.3. Synthesis of azide-containing 5-methyl-2′-deoxycytidine dC^{N3} for its incorporation into DNA

A phosphoramidite of azide-containing 5-methyl-2′-deoxycytidine dC^{N3} was prepared starting from 4-triazolothymidine 15³⁰ and 2-azidoethylamine³¹ (Scheme 3) followed by standard DMT-protection and phosphitylation using protocols for azide-containing phosphoramidites described above. Because an azide containing phosphoramidite of dC^{N3} is not suitable for large-scale production and storage, the inventors synthesised an H-phosphonate salt of 5′-O-DMT protected dC^{N3} using a standard protocol described in the past.³²

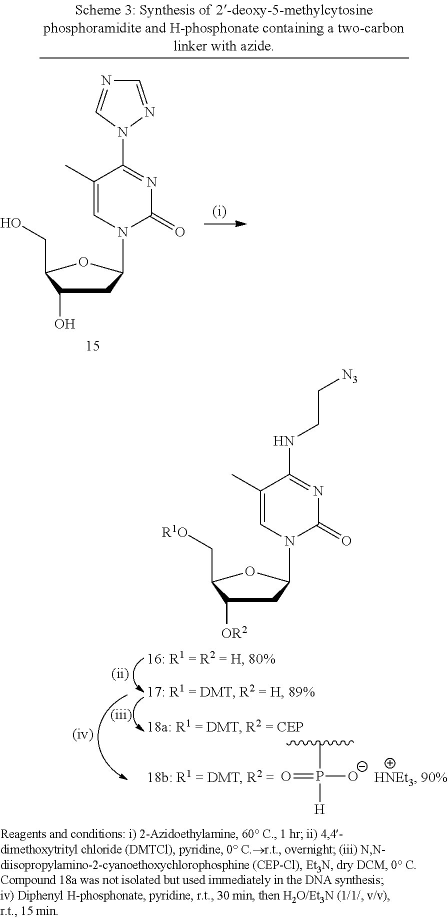

### 6.2.3.1. Synthesis of 4-(1H-1,2,4-triazol-1-yl)-2′-deoxy-5-methylcytidine (15)

Compound 15 was synthesized according to a reported procedure³⁰ (3.5 g, 58% yield).

¹H NMR (500 MHz, DMSO-d₆): δ 9.30 (s, 1H, H-6); 8.61 (s, 1H, H-9); 8.37 (s, 1H, H-11); 6.11 (dd, 1H, J_{1′,2a′}, =5.6, J_{1′,2′b}=7.3 Hz, H-1′); 5.31 (d, J=3.8 Hz, 1H, 3′-OH); 5.21 (t, J=5.3 Hz, 1H, 5′-OH); 4.27 (ddd, 1H, J₃,4′=1.9 Hz, J_{3′,2′a}=3.6 Hz, J_{3′,2′b}=5.6 Hz, H-3′); 3.90 (ddd, 1H, J_{3′,4′}=1.9 Hz, J_{4′,5′a}=3.7 Hz J_{4′,5′b}=3.5 Hz, H-4′); 3.73-3.69 (m, 2H, H-5′a); 3.64-3.60 (m, 2H, H-5′b); 2.37 (m, 1H, H-2′a); 2.30 (s, 3H, CH₃); 2.14 (ddd, 1H, J_{2′b,3′}=5.6 Hz, J_{2′b,1′}=7.3 Hz, J_{2′b,2′a}=12.1 Hz, H-2′b).

HRMS (ESI) m/z: [M+Na]⁺ Calcd for C₁₂H₁₅O₄N₅Na 316.1022. Found 316.1013.

### 6.2.3.2. Synthesis of N⁴-(2-azidoethyl)-2′-deoxy-5-methylcytidine (16)

To a suspension of compound 15 (1.9 g, 6.5 mmol), in dry acetonitrile (20 mL) was added 2-azidoethylamine (2 g, 25 mmol) and the mixture was stirred at 50° C. for 30 min. After the consumption of the starting material 15, the volatiles were evaporated in vacuo, and the crude product was purified by column chromatography over silica gel eluting with 0-30% MeOH in DCM to afford the desired product as an oil (1.6 g, 80% yield).

¹H NMR (500 MHz, DMSO-d₆): δ 7.70 (s, 1H, H-6); 7.38 (t, J=4.9 Hz, 1H, NHCH₂CH₂N₃); 6.17 (dd, 1H, J_{1′,2a′}, =5.9, J_{1′,2′b} 7.3 Hz, H-1′); 5.21 (bs, 2H, 5′-OH, 3′-OH); 4.20 (ddd, 1H, J_{3,4′}=1.9 Hz, J_{3′,2′,a}=3.6 Hz, J_{3′,2′b}=5.8 Hz, H-3′); 3.75 (ddd, 1H, J_{3,4′}=1.9 Hz, J_{4′,5′a}=3.7 Hz J_{4′,5′b}=3.5 Hz, H-4′); 3.59 (dd, 1H, J_{4′,5′a}=3.7 Hz, J_{5′a,5′b}=11.8 Hz, H-5′a); 3.53 (dd, 1H, J_{4′,5′b}=3.5 Hz, J_{5′a,5′b}=11.8 Hz, H-5′b); 3.52-3.50 (m, 2H, NHCH₂CH₂N₃); 3.47-3.45 (m, 2H, NHCH₂CH₂N₃); 2.08 (ddd, 1H, J_{2′a,3′}=3.6 Hz, J_{2′a,1′}=5.9 Hz, J_{2′a,2′b}=13.2 Hz, H-2′a); 1.97 (ddd, 1H, J_{2′b,3′}=5.8 Hz, J_{2′b,1′}=7.3 Hz, J_{2′b,2′a}=13.2 Hz, H-2′b); 1.87 (s, 3H, CH₃).

HRMS (ESI) m/z: [M+H]⁺ Calcd for C₁₂H₁₉N₆O₄ 311.1468. Found 311.1458.

### 6.2.3.3. Synthesis of 5′-O-(4,4′-dimethoxytrityl)-N⁴-(2-azidoethyl)-2′-deoxy-5-methylcytidine (17)

To a stirring solution of compound 16 (0.47 g, 1.5 mmol) in dry pyridine (10 mL), 4,4′-dimethoxytrityl chloride (0.62 g, 1.8 mmol) was added at 0° C. and the mixture was stirred at r.t. overnight under argon. After consumption of the starting nucleoside, water (1 mL) was added. Solvents were evaporated in vacuo and the residue was dissolved in 50 mL DCM, washed with brine (2×10 mL). Organic layer was dried over anhydrous sodium sulfate, filtered, and concentrated in vacuo. The crude product was purified by column chromatography over silica gel treated with 10% Et₃N in DCM and eluted with 20% MeOH in DCM to afford the desired product as a foam (0.83 g, 89% yield).

¹H NMR (500 MHz, DMSO-d₆): δ 7.70 (s, 1H, H-6); 7.40-7.38 (m, 3H, H₂″, NHCH₂CH₂N₃); 7.31 (m 2H, H-3″); 7.27-7.22 (m, 5H, H-4″ and H-2′″); 6.90-6.6.89 (m, 4H, H-3′″); 6.23 (dd, 1H, J_{1′,2a′}, =5.9, J_{1′,2′b} 7.3 Hz, H-1′); 5.30 (d, J=4.4 Hz, 1H, 3′-OH); 4.29 (ddd, 1H, J₃,4′=2.2 Hz, J_{3′,2′,a}=3.6 Hz, J_{3′,2′b}=5.8 Hz, H-3′); 3.88 (ddd, 1H, J₃,4′=2.2 Hz, J_{4′,5′a}=3.7 Hz J_{4′,5′b}=3.5 Hz, H-4′); 3.73 (s, 6H, OCH₃); 3.51 (t, J=5.5 Hz, 2H, NHCH₂CH₂N₃); 3.48-3.45 (m, 2H, NHCH₂CH₂N₃); 3.23-3.16 (m, 2H, H₅′); 2.20-2.16 (m, 1H, H-2′a); 2.15-2.08 (m, 1H, H-2′b); 1.51 (s, 3H, CH₃).

HRMS (ESI) m/z: [M+Na]⁺ Calcd for C₃₃H₃₆N₆O₆Na 635.2594. Found 635.2576.

### 6.2.3.4. Synthesis of 5′-O-(4,4′-dimethoxytrityl)-N⁴-(2-azidoethyl)-3′-O—(N,N-diisopropylamino-2-cyanoethoxyphosphanyl)-2′-deoxy-5-methylcytidine (18a)

Synthesis of compound 18a was performed in the same way as described for compound 14c. The concentration of azide phosphoramidite in DCM solution was calculated to be 0.14 M based on UV absorption of DMT-cation at 504 nm (ε=76,000 L·mol⁻¹·cm⁻¹) that was released from an aliquot of compound 18a by treatment with 3% dichloroacetic acid in DCM.

¹H NMR (500 MHz, CDCl₃): δ 7.73, 7.67 (2s, 1H, H-6); 8.27, 8.24 (2s, 1H, H-2); 7.41-7.38 (m, 2H, H-2″); 7.30-7.19 (m, 6H, H-3″, H-4″ and H-2′″); 6.82-6.79 (m, 5H, H-3′″ and H-2′″); 6.44-6.39 (m, 1H, H-1′); 5.42-5.38 (m, 1H, H-3′); 4.62-4.59 (m, 1H, H-4′); 4.12-4.07 (m, 2H, CH₂CHCN); 3.77, 3.76 (2s, 6H, OCH₃); 3.73-3.68 (m, 2H, NCH₂CH₂N₃); 3.56-3.46 (m, 5H, NCHCH₃, NCH₂CH₂N₃, H-5′a); 3.30-3.26 (m, 1H, H-5′b); 2.75-2.72 (m, 1H, H-2′b); 2.59 (t, J=6.5 Hz, 2H, CH₂CH₂CN); 2.29-2.21 (m, 1H, H-2′a); 1.45, 1.42 (2s, CH₃); 1.14-1.12 (m, 12H, NCHCH₃).

³¹P NMR (202.5 MHz, DMSO-d₆, ref. 85% H₃PO₄) δ 148.87, 148.32.

IR ATR (cm⁻¹): 3275.67, 2971.79, 2935.55, 2907.48, 2874.94, 2100.98, 1668.30, 1634.24, 1609.40, 1511.06, 1464.34, 1253.62, 1180.73, 735.74.

### 6.2.3.5. Synthesis of triethylammonium 5′-O-(4,4′-dimethoxytrityl)-N⁴-(2-azidoethyl)-2′-deoxy-5-methylcytidine-3′-H-phosphonate (18b)

To a stirring solution of compound 17 (2 g, 3.2 mmol) in dry pyridine (20 mL), diphenyl H-phosphonate (4.3 mL, 22 mmol) was added in one portion, and the mixture was stirred at r.t. for 30 min. After consumption of the starting nucleoside, water (1 mL) and Et₃N (1 mL) were added to quench the reaction. Reaction mixture was stirred for 15 min. Solvents were evaporated in vacuo and the residue was partitioned between DCM (50 mL) and 5% sodium bicarbonate (10 mL). Organic phase was extracted twice with 5% sodium bicarbonate (10 mL), dried over anhydrous sodium sulfate, filtered, and concentrated in vacuo to produce an oil. The crude product was purified by column chromatography over silica gel treated with 10% Et₃N in DCM and eluted with 0-40% MeOH in DCM to afford the desired product as a foam (2.10 g, 90% yield). Compound 18b was found to be hygroscopic and was stored in airtight container under argon.

¹H NMR (500 MHz, DMSO-d₆): δ 7.50 (s, 1H, H-6); 7.48-7.44 (m, 1H, NH); 7.40-7.38 (m, 2H, H-2″); 7.33-7.29 (m, 2H, H-3″); 7.28-7.22 (m, 5H, H-4″ and H-2′″); 6.92-6.88 (m, 4H, H-3′″); 6.23 (dd, 1H, J_{1′,2a′}=1.40, J_{1′,2′b} 7.3 Hz, H-1′); 4.75-4.71 (m, 1H, H-3′); 4.05 (td, 1H, J=3.20, 3.60 Hz H-4′); 3.74 (s, 6H, OCH₃); 3.52 (t, 2H, J=5.5 Hz, NCH₂CH₂N₃); 3.49-3.46 (m, 2H, NCH₂CH₂N₃); 3.26-3.22 (m, 2H, H-5′); 3.00 (q, 6H, J=7.25 Hz, CH₂); 2.31 (ddd, 1H, J_{2′b,3′}=3.05 Hz, J_{2′b,1′}=5.60 Hz, J_{2′a,2′b}=13.35 Hz, H-2′b); 2.20 (ddd, 1H, J_{2′a,3′}=5.30 Hz, J_{2′a,1′}=6.60 Hz, J_{2′a,2′b}=13.35 Hz, H-2′a); 1.48 (s, 3H, CH₃); 1.16 (t, 3H, J=7.25 Hz, CH₃).

³¹P NMR (202.5 MHz, DMSO-d₆, ref. 85% H₃PO₄): δ 0.04 HRMS (ESI) m/z: [M−H]⁻ Calcd for C₃₃H₃₆O₈N₆ 675.2338. Found 675.2344.

### 6.2.4. Synthesis of Modified Carbazole Nucleosides

Synthesis of carbazole nucleosides of dH^{E} series (FIG. 2H) started from 3-iodocarbazole 19 and Hoffer's chlorosugar 1 which coupled in the presence of K-^{t}BuO in 1,4-dioxane at room temperature (Scheme 4). A subsequent Sonogashira reaction with TMS or TIPS-protected acetylene catalyzed by Pd(PPh₃)₄ and CuI resulted in compounds 21a-b. Removal of 3′,5′-O-toluoyl and TMS protecting groups in 21a was accomplished by K₂CO₃ in MeOH and for selective deprotection of 3′,5′-O-toluoyls in 21b we used 30% aq. NH₃ in MeOH at rt for 3 days. These deprotection conditions when applied on compound 20 resulted in removal of iodine from the carbazole ring. To preserve iodine in the aromatic cycle the inventors used guanidine in MeOH at 0-5° C. and compound 22c was obtained in 80% yield. Conversion into 5′-O-DMT derivatives followed by phosphitylation to produce phosphoramidites 24a-c or loading of TIPS-protected 3-ethenylcarbazole 23b onto CPG-support providing compound 25 were performed using established protocols.

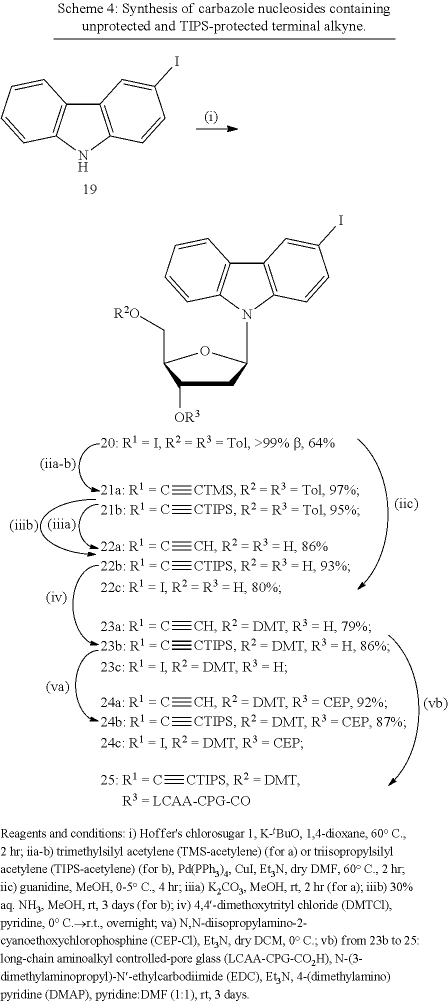

### 6.2.4.1. Synthesis of 3-iodocarbazole (19)

3-Iodocarbazole was prepared according to a reported procedure.³³

### 6.2.4.2. Synthesis of 9-[2-deoxy-3,5-bis-O-(4-methylbenzoyl)-β-D-erythro-pentofuranosyl]-3-iodo-9H-carbazole (20)

To a solution of 3-iodocarbazole (3.2 g, 10 mmol) in 100 mL of 1,4-dioxane was added K-^{t}BuO (1.46 g, 13 mmol) followed by Hoffer's chlorosugar 1 (4.19 g, 10.8 mmol) and the solution was stirred at rt for 2 hr. After the consumption of the starting materials as observed by TLC, the reaction mixture was diluted with EtOAc (100 mL) and washed with water. Organic layer was separated, dried over anhydrous sodium sulfate, filtered, and concentrated in vacuo. The crude product was purified by column chromatography over silica gel eluting with 0-10% EtOAc in hexane to afford the desired product as a foam (yield 4.5 g, 64%, with purity over 99% as β-nucleoside determined by ¹H NMR).

¹H NMR (500 MHz, CDCl₃): δ 8.38 (d, J=1.4 Hz, 1H, H-4); 8.06-8.00 (m, 5H, H-3″, H-3′″ and H-6); 7.66-7.64 (m, 1H, H-8); 7.45-7.41 (m, 2H, H-2 and H-1); 7.34-7.31 (m, 4H, H-2″ and H-2′″); 7.27-7.24 (m, 2H, H-7 and H-5); 6.69 (dd, 1H, J_{1′,2a′}, =5.8, J_{1′,2′b}=9.3 Hz, H-1′); 5.85 (ddd, 1H, J_{3′,4′}=1.8 Hz, J_{3′,2′a}=3.7 Hz, J_{3′,2′b}=5.7 Hz, H-3′); 4.92 (dd, 1H, J_{4′,5′a}=2.7 Hz, J_{5′a,5′b}=12.2 Hz, H-5′a); 4.83 (dd, 1H, J_{4′,5′b}=3.4 Hz, J_{5′a,5′b}=12.2 Hz, H-5′b); 4.59 (ddd, 1H, J_{3′,4′}=1.8 Hz, J_{4′,5′a}=2.7 Hz J_{4′,5′b}=3.4 Hz, H-4′); 3.18 (ddd, 1H, J_{2′b,3}=5.7 Hz, J_{2′b,1′}=9.3 Hz, J_{2′b,2′a}=14.5 Hz, H-2′b); 2.51 (ddd, 1H, J_{2′a,3′}=3.7 Hz, J_{2′a,1′}=5.8 Hz, J_{2′a,2′b}=14.5 Hz, H-2′a); 2.50, 2.49 (2s, 6H, CH₃).

HRMS (ESI) m/z: [M+Na]⁺ Calcd for C₃₃H₂₈INNaO₅ 668.0904. Found 668.0895.

### 6.2.4.3. General Procedure for Sonogashira Reaction of Compound 20 with Protected Acetylenes

In a 100 mL round bottom flask, compound 20 (1.0 g, 3.4 mmol), tetrakis(triphenylphosphine)palladium (0) (0.185 g, 0.16 mmol) and CuI (0.06 g, 3.1 mmol) were added. The flask was sealed with septum and carefully degassed in vacuo and then purged with argon. Dry DMF (15 mL) was added via syringe, followed by protected acetylene (4 eqv. with reference to compound 20) and triethylamine (3.6 mmol). The content of the flask was stirred at 60° C. for 2 hr until compound 20 was completely consumed based on TLC analysis of the reaction mixture. The contents of the flask were diluted with EtOAc (30 mL), washed with distilled water (10 mL) followed by brine (10 mL). The organic layer was separated and dried over anhydrous sodium sulfate, filtered, and concentrated in vacuo. The crude product was purified by column chromatography over silica gel eluting with 0-15% EtOAc in hexane to afford the desired product as a foam.

### 6.2.4.3.1. Characterization of 9-[2-deoxy-3,5-bis-O-(4-methylbenzoyl)-β-D-erythro-pentofuranosyl]-3-[(trimethylsilyl)ethynyl]-9H-carbazole (21a)

Yield: 0.93 g, 97%.

¹H NMR (500 MHz, CDCl₃): δ 8.19 (d, J=1.2 Hz, 1H, H-4); 8.04-7.99 (m, 5H, H-3″, H-3′″ and H-6); 7.65-7.64 (m, 1H, H-8); 7.53 (d, J=8.5 Hz, 1H, H-1); 7.33-7.28 (m, 5H, H-2″, H-2′″ and H-2); 7.23-7.20 (m, 2H, H-7 and H-5); 6.68 (dd, 1H, J_{1′,2a′}=5.8, J_{1′,2′b} 9.3 Hz, H-1′); 5.83 (ddd, 1H, J_{3′,4′}=1.8 Hz, J_{3′,2′a}=3.7 Hz, J_{3′,2′b}=5.6 Hz, H-3′); 4.88 (dd, 1H, J_{4′,5′a}=2.8 Hz, J_{5′a,5′b}=12.2 Hz, H-5′a); 4.81 (dd, 1H, J_{4′,5′b}=3.5 Hz, J_{5′a,5′b}=12.2 Hz, H-5′b); 4.56 (ddd, 1H, J_{3′,4′}=1.8 Hz, J_{4′,5′a}=2.8 Hz J_{4′,5′b}=3.5 Hz, H-4′); 3.18 (ddd, 1H, J_{2′b,3}=5.6 Hz, J_{2′b,1′}=9.3 Hz, J_{2′b,2′a}=14.5 Hz, H-2′b); 2.37 (ddd, 1H, J_{2′a,3′}=3.7 Hz, J_{2′a,1′}=5.8 Hz, J_{2′a,2′b}=14.5 Hz, H-2′a); 2.45 (s, 6H, CH₃); 0.29 (s, 9H, Si(CH₃)₃).

HRMS (ESI) m/z: [M+Na]⁺ Calcd for C₃₈H₃₇NO₅SiNa 638.2333. Found 638.2324.

### 6.2.4.3.2. Characterization of 9-[2-deoxy-3,5-bis-O-(4-methylbenzoyl)-3-D-erythro-pentofuranosyl]-3-[(triisopropylsilyl)ethynyl]-9H-carbazole (21b)

Yield: 1.03 g, 95%.

¹H NMR (500 MHz, DMSO-d₆): δ 8.32 (s, 1H, H-4); 8.26 (d, J=7.6 Hz, 1H, H-5); 8.03-7.99 (m, 4H, H-3″); 7.81 (d, J=8.1 Hz, 1H, H-8); 7.75 (d, J=8.5 Hz, 1H, H-1); 7.40-7.37 (4H, H₃′″); 7.30-7.23 (m, 2H, H-6 and H-7); 7.13 (d, J=8.5 Hz, 1H, H-2); 6.90 (dd, 1H, J_{1′,2a′}, =6.5, J_{1′,2′b} 8.4 Hz, H-1′); 5.88-5.85 (m, 1H, H-3′); 4.86 (dd, 1H, J_{4′,5′a}=2.2 Hz, J_{5′a,5′b}=12.2 Hz, H-5′a); 4.68 (dd, 1H, J_{4′,5′b}=3.7 Hz, J_{5′a,5′b}=12.2 Hz, H-5′b); 4.55 (ddd, 1H, J_{4′,5′a}=2.2 Hz, J₃,4′=2.6 Hz, J_{4′,5′b}=3.7 Hz, H-4′); 3.08 (ddd, 1H, J_{2′b,3}′=7.5 Hz, J_{2′b,1′}=8.4 Hz, J_{2′b,2′a}=14.8 Hz, H-2′b); 2.60 (ddd, 1H, J_{2′a,3′}=2.5 Hz, J_{2′a,1′}=6.5 Hz, J_{2′a,2′b}=14.8 Hz, H-2′a); 2.42, 2.41 (s, 6H, CH₃); 1.13 (s, 21H, Si(CHC₂H₆)₃).

HRMS (ESI) m/z: [M+Na]⁺ Calcd for C₄₄H₄₉O₅SiNNa 722.3272. Found 722.3259.

### 6.2.4.4. Synthesis of 9-[2-deoxy-β-D-erythro-pentofuranosyl]-3-ethynyl-9H-carbazole (22a)

To a solution of compound 21a (0.96 g, 1.6 mmol) in methanol (30 mL), was added K₂CO₃ (0.47 g, 3.4 mmol) and the mixture was stirred for 2 hr at rt. After the consumption of the starting material as observed by TLC, the solvent was evaporated, and the residue was dissolved in 100 mL DCM. The DCM solution was washed with distilled water (20 mL) followed by brine (10 mL). The organic layer was dried over anhydrous sodium sulfate, filtered, and concentrated in vacuo. The crude product was purified by column chromatography over silica gel eluting with 0-5% MeOH in DCM to afford the desired product as a foam (0.55 g, 86% yield).

¹H NMR (500 MHz, DMSO-d₆): δ 8.34 (d, J=1.4 Hz, 1H, H-4); 8.21 (d, J=7.7 Hz, 1H, H-5); 7.81 (s, 1H, H-8); 7.80 (s, 1H, H-1); 7.50 (dd, 1H, J=1.6, 8.5 Hz, H-7); 7.47-7.43 (m, 1H, H₂); 7.26-7.24 (m, 1H, H₆); 6.68 (dd, 1H, J_{1′,2a′}, =6.5, J_{1′,2′b}=8.5 Hz, H-1′); 5.38 (d, J_{3′-OH,3′}=4.6 Hz, 1H, 3′-OH); 5.04 (t, J_{5′-OH,5′}=5.2 Hz, 1H, 5′-OH); 4.48-4.44 (m, 1H, H-3′); 4.06 (s, 1H, H-14); 3.87 (ddd, 1H, J_{4′,5′a}=3.3 Hz, J_{3′,4′}=4.1 Hz, J_{4′,5′b}=7.9 Hz, H-4′); 3.79-3.70 (m, 2H, H-5′); 2.64 (ddd, 1H, J_{2′b,3′}=6.5 Hz, J_{2′b,1′}′=8.5 Hz, J_{2′b,2′a}=13.5 Hz, H-2′b); 2.12 (ddd, 1H, J_{2′a,3′}=2.8 Hz, J_{2′a,1′}=6.5 Hz, J_{2′a,2′b}=13.5 Hz, H-2′a).

HRMS (ESI) m/z: [M+Na]⁺ Calcd for C₁₉H₁₇O₃NNa 330.1101. Found 330.1097.

### 6.2.4.5. Synthesis of 9-[2-deoxy-β-D-erythro-pentofuranosyl]-3-[(triisopropylsilyl)ethynyl]-9H-carbazole (22b)

Compound 21a (1.05 g, 1.50 mmol) was dissolved in MeOH (500 mL) and 28% aq. ammonia (50 mL) was added in one portion. Reaction mixture was stirred at room temperature for 48 h, evaporated in vacuo, co-evaporated with H₂O (2×200 mL). The crude product was purified by column chromatography over silica gel eluting with 0-5% MeOH in DCM to afford the desired product as a foam (0.65 g, 93% yield).

¹H NMR (500 MHz, DMSO-d₆): δ 8.33 (s, 1H, H-4); 8.26 (d, J=7.7 Hz, 1H, H-5); 7.82-7.80 (m, 2H, H-1 and H-8); 7.50-7.44 (m, 2H, H-2 and H-7); 7.25 (dd, J=7.5, 7.40 Hz, 1H, H-6); 6.68 (dd, 1H, J_{1′,2a′}, =7.1, J_{1′,2′b}=7.7 Hz, H-1′); 5.38 (d, J_{3′OH},3′=4.6 Hz, 1H, 3′-OH); 5.03 (t, J_{5′OH,5′}=5.2 Hz, 1H, 5′-OH); 4.48-4.44 (m, 1H, H-3′); 3.87 (ddd, 1H, J_{4′,5′a}=3.2 Hz, J₃,4′=4.4 Hz, J_{4′,5′b}=9.0 Hz, H-4′); 3.79-3.70 (m, 2H, H-5′); 2.64 (ddd, 1H, J_{2′b,3′}=6.5 Hz, J_{2′b,1′}=7.7 Hz, J_{2′b,2′a}=13.6 Hz, H-2′b); 2.12 (ddd, 1H, J_{2′a,3}=2.8 Hz, J_{2′a,1′}=7.1 Hz, J_{2′a,2′b}=13.6 Hz, H-2′a); 1.14 (s, 21H, Si(CHC₂H₆)₃).

¹³C NMR (125.7 MHz, DMSO-d₆): δ 139.14 (1C, C11); 138.60 (1C, C10); 129.53 (1C, C7); 126.48 (1C, C2) 123.82 (1C, C5); 123.07 (1C, C9); 122.48 (1C, C12); 120.79 (1C, C8); 120.12 (1C, C6); 113.28 (1C, C3); 111.50 (2C, C1 and C4); 108.56 (1C, C13); 87.44 (1C, C14); 86.51 (1C, C4′); 84.01 (1C, C1′); 70.08 (1C, C3′); 61.24 (1C, C5′); 37.43 (1C, C2′); 18.59 (3C, SiCH); 10.84 (6C, CH₃).

HRMS (ESI) m/z: [M+Na]⁺ Calcd for C₂₈H₃₇O₃NSiNa 486.2435. Found 486.2432.

### 6.2.4.6. Synthesis of 9-[2-deoxy-β-D-erythro-pentofuranosyl]-3-iodo-9H-carbazole (22c)

Guanidine hydrochloride (3 g, 31 mmol) was suspended in liquid ammonia (10 mL) and finely powered NaOH (2.5 g, 62.5 mmol) was added. Vacuum was applied occasionally to prevent ammonia evaporation. The reaction mixture was stirred for 1.5 hour. Precipitated NaCl was filtered, and ammonia solution was evaporated by passing N₂ through solution for 45 min. The oily residue (guanidine) obtained was dried in vacuo.

Compound 20 (4.63 g, 7.2 mmol) was dissolved in MeOH (500 mL) and guanidine (0.85 g, 14.3 mmol) was added in one portion. Reaction mixture was stirred at 0° C. for 4 hrs and after consumption of nucleoside 20, reaction mixture was evaporated in vacuo. The crude product was purified by column chromatography over silica gel eluting with 0-5% MeOH in DCM to afford the desired product as a foam (2.3 g, 80% yield). The product was further converted to the DMT-protected phosphoramidite 24c using general protocols. Characterisation of compound 24c is given below.

### 6.2.4.7. General Protocol for 5′-O-4,4′-dimethoxytritylation of Modified Carbazole Nucleosides

To a stirring solution of a nucleoside (0.32 g, 1.0 mmol) in dry pyridine (10 mL), 4,4′-dimethoxytrityl chloride (0.67 g, 1.2 mmol) was added at 0° C. and the mixture was stirred at r.t. overnight under argon. After consumption of the starting nucleoside, water (1 mL) was added. Solvents were evaporated in vacuo and the residue was dissolved in 50 mL DCM and washed with brine (2×10 mL). Organic layer was dried over anhydrous sodium sulfate, filtered, and concentrated in vacuo. The crude product was purified by column chromatography over silica gel treated with 10% Et₃N in DCM and eluted with 10% acetone in DCM to afford the desired product as a foam.

### 6.2.4.7.1. Characterization of 9-[2-deoxy-5-O-(4,4′-dimethoxytrityl)-3-D-erythro-pentofuranosyl]-3-ethynyl-9H-carbazole (23a)

Yield: 0.5 g, 79%.

¹H NMR (500 MHz, CDCl₃): δ 8.36 (d, J=1.3 Hz, 1H, H-4); 8.04-8.02 (m, 1H, H-5); 7.65-7.64 (m, 1H, H-8); 7.66-7.63 (m, 1H, H-4″); 7.59 (d, 1H, J=8.5 Hz, 1H, H-1); 7.51-7.48 (m, 2H, H-2″); 7.40-7.37 (m, 4H, H-2′″); 7.36-7.34 (m, 1H, H-2); 7.30-7.28 (m, 3H, H-3″ and H-6); 7.25-7.22 (m, 2H, H-2, H-7); 6.84-6.81 (m, 4H, H-3′″); 6.62 (dd, 1H, J_{1′,2a′}, =6.5, J_{1′,2′b}=8.5 Hz, H-1′); 4.77 (ddd, 1H, J_{3′,4′}=1.8 Hz, J_{3′,2′a}=3.2 Hz, J_{3′,2′b}=7.1 Hz, H-3′); 4.08 (ddd, 1H, J_{3′,4′}=1.8 Hz, J_{4′,5′a}=2.8 Hz J_{4′,5′b}=3.8 Hz, H-4′); 3.79 (s, 3H, OCH₃); 3.78 (s, 3H, OCH₃); 3.56 (d, 2H, J=3.8 Hz, H-5′); 3.05 (s, 1H, H-14); 2.90 (ddd, 1H, J_{2′b,3′}=7.1 Hz, J_{2′b,1′}=8.5 Hz, J_{2′b,2′,a}=13.9 Hz, H-2′b); 2.22 (ddd, 1H, J_{2′a,3′}=3.2 Hz, J_{2′a,1′}=6.5 Hz, J_{2′a,2′b}=13.9 Hz, H-2′a).

HRMS (ESI) m/z: [M+Na]⁺ Calcd for C₄₀H₃₅O₅NNa 632.2407. Found 632.2401.

### 6.2.4.7.2. Characterization of 9-[2-deoxy-5-O-(4,4′-dimethoxytrityl)-3-D-erythro-pentofuranosyl]-3-[(triisopropylsilyl)ethynyl]-9H-carbazole (23b)

Yield: 0.45 g, 86%

¹H NMR (500 MHz, CDCl₃): δ 8.33 (d, J=1.2 Hz, 1H, H-4); 8.27 (d, J=7.5 Hz, 1H, H-5); 7.81-7.83 (m, 2H, H₁ and H₈); 7.44-7.41 (m, 2H, H-2″); 7.30-7.25 (m, 7H, H-3″, H-4″ and H-2′″); 7.24-7.20 (m, 3H, H-7, H-2 and H-6); 6.86-6.84 (4H, H-3′″); 6.73 (dd, 1H, J_{1′,2a′}=6.9, J_{1′,2′b}=8.1 Hz, H-1′); 5.48 (d, J=4.9 Hz, 1H, 3′-OH); 4.62 (ddd, 1H, J_{3′,4′}=1.8 Hz, J_{3′,2′a}=3.7 Hz, J_{3′,2′b}=5.6 Hz, H-3′); 4.0 (ddd, 1H, J_{3′,4′}=1.8 Hz, J_{4′,5′a}=4.9 Hz, J_{4′,5′b}=2.3 Hz, H-4′); 3.72 (s, 6H, OCH₃); 3.41 (dd, 1H, J_{4′,5′a}=4.9 Hz, J_{5′a,5′b}=10.5 Hz, H-5′a); 3.36 (dd, 1H, J_{4′,5′b}=2.3 Hz, J_{5′a,5′b}=10.5 Hz, H-5′b); 2.71 (ddd, 1H, J_{2′b,3}=5.6 Hz, J_{2′b,1′}=8.1 Hz, J_{2′b,2′a}=13.6 Hz, H-2′b); 2.20 (ddd, 1H, J_{2′a,3′}=3.7 Hz, J_{2′a,1′}=6.9 Hz, J_{2′a,2′b}=13.6 Hz, H-2′a); 1.14 (s, 21H, Si(CHC₂H₆)₃).

HRMS (ESI) m/z: [M+Na]⁺ Calcd for C₄₉H₅₅O₅NSiNa 788.3742. Found 788.3732.

### 6.2.4.8. Synthesis of 9-[3-O—(N,N-diisopropylamino-2-cyanoethoxyphosphanyl)-2-deoxy-5-O-(4,4′-dimethoxytrityl)-β-D-erythro-pentofuranosyl]-3-ethynyl-9H-carbazole (24a)

Compound 24a was synthesized using the same procedure as for compound 14a.

Yield: 0.57 g, 92%.

¹H NMR (500 MHz, DMSO-d₆): δ 8.35 (s, 1H, H-4); 8.23-8.22 (m, 1H, H-5); 7.83-7.80 (m, 2H, H-1 and H-8); 7.45-7.41 (m, 2H, H-2″); 7.31-7.20 (m, 10H, H-3″, H-4″, H-2′″, H-7, H-2 and H-6); 6.86-6.84 (m, 2H, H-3′″); 6.83-6.80 (m, 2H, H-3′″); 6.78 (dd, 1H, J_{1′,2a′}=6.8, J_{1′,2′b}=8.2 Hz, H-1′); 4.96-4.83 (m, H-3′); 4.14-4.09 (m, H-4′); 4.06 (s, C—CH); 3.72, 3.71, 3,70 (3s, 6H, OCH₃); 3.66-3.60 (m, 2H, CH₂CH₂CN); 3.59-3.50 (m, 2H, NCHCH₃); 3.46-3.37 (m, 2H, H-5′); 2.86-2.79 (m, 1H, H-2′b); 2.74, 2.66 (2t, J=5.9 Hz, 2H, CH₂CH₂CN); 2.46-2.34 (m, 1H, H-2′a); 1.14-1.08 (m, 10H, NCHCH₃); 0.99 (d, J=6.8 Hz, 2H, NCHCH₃).

HRMS (ESI) m/z: [M+Na]⁺ Calcd for C₄₉H₅₂O₆N₃Na 832.3486. Found 832.3469.

### 6.2.4.9. Synthesis of 9-[3-O—(N,N-diisopropylamino-2-cyanoethoxyphosphanyl)-2-deoxy-5-O-(4,4′-dimethoxytrityl)-β-D-erythro-pentofuranosyl]-3-[(triisopropylsilyl)ethynyl]-9H-carbazole (24b)

Compound 24b was synthesized using the same procedure as for compound 14a.

Yield: 1.2 g, 87%.

¹H NMR (500 MHz, DMSO-d₆): δ 8.33 (s, 1H, H-4); 8.27-8.25 (m, 1H, H-5); 7.84-7.80 (2H, H-1 and H-8); 7.45-7.41 (m, 2H, H-2″); 7.31-7.17 (m, 10H, H-3″, H-4″, H-2′″, H-7, H-2 and H-6); 6.86-6.81 (m, 4H, H-3′″); 6.79 (dd, 1H, J_{1′,2a′}=7.5, J_{1′,2′b}=7.6 Hz, H-1′); 4.99-4.86 (m, 1H, H-3′); 4.15-4.08 (m, H-4′); 3.71 (s, 6H, OCH₃); 3.67-3.61 (m, 2H, NCHCH₃); 3.59-3.50 (m, 2H, CH₂CH₂CN); 3.46-3.37 (m, 2H, H-5′); 2.85-2.78 (m, 1H, H-2′b); 2.75, 2.66 (2t, J=5.90 Hz, 2H, CH₂CH₂CN); 2.47-2.34 (m, 1H, H-2′a); 1.14-1.12 (m, 30H, SiCHCH₃×3 and NCHCH₃×2); 0.99-0.98 (d, m, SiCHCH₃×3).

HRMS (ESI) m/z: [M+Na]⁺ Calcd for C₅₈H₇₂O₆N₃NaPSi 988.4826. Found 988.4802.

### 6.2.4.10. Synthesis of 9-[3-O—(N,N-diisopropylamino-2-cyanoethoxyphosphanyl)-2-deoxy-5-O-(4,4′-dimethoxytrityl)-β-D-erythro-pentofuranosyl]-3-iodo-9H-carbazole (24c)

Compound 24c was synthesized using the same procedure as for compound 14a.

¹H NMR (500 MHz, DMSO-d₆): δ 8.63, 8.57 (2s, 1H, H-4); 8.21-8.14 (m, 1H, H-5); 7.82-7.81 (m, 1H, H-1); 7.69 (d, 1H, J=8.50 Hz, H-8); 7.47-7.38 (m, 2H, H-2″); 7.33-7.21 (m, 9H, H-3″, H-4″, H-2′″, H-2 and H-6); 7.14-7.10 (m, 1H, H-7); 6.87-6.82 (m, 4H, H-3′″); 6.77 (dd, 1H, J=6.95, 14.5 Hz, H-1′); 4.98-4.85 (m, H-3′); 4.17-4.08 (m, H-4′); 3.72 (3s, 6H, OCH₃); 3.70-3.61 (m, 2H, CH₂CH₂CN); 3.59-3.50 (m, 2H, NCHCH₃); 3.46-3.40 (m, 2H, H-5′); 2.91-2.78 (m, 1H, H-2′b); 2.74, 2.66 (2t, J=5.80 Hz, 2H, CH₂CH₂CN); 2.46-2.33 (m, 1H, H-2′a); 1.14-1.08 (m, 10H, NCHCH₃); 1.00 (d, J=6.5 Hz, 2H, NCHCH₃).

HRMS (ESI) m/z: [M+Na]⁺ Calcd for C₄₇H₅₂O₆IN₃P 912.2638. Found 912.2622.

### 6.2.4.11. Synthesis of 9-[2-deoxy-5-O-(4,4′-dimethoxytrityl)-3-D-erythro-pentofuranosyl]-3-[(triisopropylsilyl)ethynyl]-9H-carbazole bound to CPG support (25)

Compound 23b was covalently attached to the CPG support using procedure described for compound 4. The load on the CPG 25 was determined to be 48 μmol/g based on UV absorption of DMT-cation at 504 nm (ε=76,000 L·mol⁻¹·cm⁻¹) that was released from an aliquot of compound by treatment with 3% dichloroacetic acid in DCM.

### 6.2.5. Synthesis of H-phosphonate of 5-azidomethyl-2′-deoxyuridine

5-Azidomethyl-5′-O-(4,4′-dimethoxytrityl)-2′-deoxyuridine (26) was synthesized according to known procedures³⁴⁻³⁷ starting from thymidine. For incorporation into DNA, this azido-containing nucleoside was converted into its 3′-H-phosphonate 27 using diphenyl H-phosphonate by a general procedure used for the synthesis of compound 18b.³²

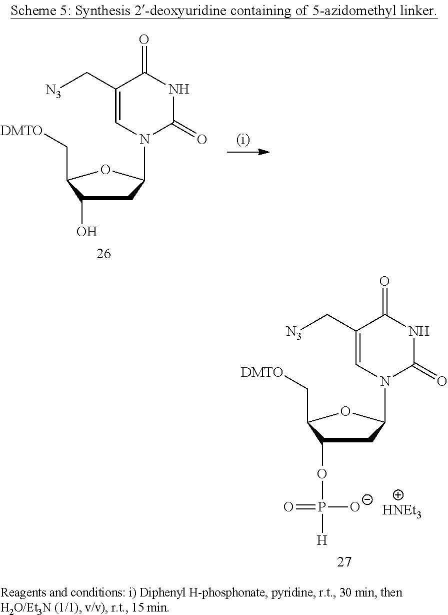

### 6.2.5.1.1. Characterization of triethylammonium 5-azidomethyl-5′-O-(4,4′-dimethoxytrityl)-2′-deoxyuridine-3′-H-phosphonate (27)

Yield: 0.9 g, 97%.

¹H NMR (500 MHz, DMSO-d₆): δ 7.78 (s, 1H, H-6); 7.39-7.37 (m, 2H, H-2″); 7.33-7.30 (m, 2H, H-3″); 7.27-7.24 (m, 5H, H-4″ and H-2′″); 6.90-6.88 (m, 4H, H-3′″); 6.18 (app t, 1H, J=6.7 Hz, H-1′); 4.77-4.71 (m, 1H, H-3′); 4.07 (td, 1H, J=2.80, 3.50 Hz H-4′); 3.74 (s, 6H, OCH₃); 3.66 (s, 2H, CH₂N₃); 3.28 (dd, 2H, dd, J=4.85, 10.45 Hz, H-5′a); 3.21 (dd, 2H, dd, J=2.90, 10.45 Hz, H-5′b); 2.99 (q, 6H, J=7.24 Hz, CH₂); 2.39-2.29 (m, 2H, H-2′); 1.16 (t, 3H, J=7.24 Hz, CH₃).

³¹P NMR (202.5 MHz, DMSO-d₆, ref. 85% H₃PO₄): δ 0.02

HRMS (ESI) m/z: [M−H]⁻ Calcd for C₃₃H₃₆O₈N₆ 648.1865. Found 648.1871.

### 6.3. Example 2: Oligonucleotide Synthesis and Purification

Oligonucleotides were prepared on a MerMade-4 DNA/RNA synthesizer (BioAutomation, USA) on a 5 μmol scale using standard manufacturer's protocol. Coupling times of modified phosphoramidites were increased from 2 to 10 min.

To prevent a Staudinger reaction between the azide and phosphoramidite present in one molecule, the inventors did not purify the products 5, 14c and 18a or concentrate them. Instead, these compounds were used directly for the oligonucleotide synthesis. The concentration of reagents 5, 14c and 18a in DCM was found to be 0.18, 0.20 and 0.14 M respectively, determined by UV absorption of the 4,4′-dimethoxytrityl cation released upon treatment of an aliquot of this sample with a 3% solution of dichloroacetic acid in dichloromethane.

For incorporation of an H-phosphonate of nucleoside 18b into DNA, H-phosphonate was dissolved in a mixture of dry pyridine and freshly distilled ACN (3:7, v/v). During the coupling step, the H-phosphonate solution was delivered simultaneously with a solution of 0.5M pivaloyl chloride in acetonitrile in 2:3 ratio (v/v) onto the CPG-support containing growing chain of DNA. Coupling time was 6 min for each addition that was then repeated once. The unreacted 5′-hydroxyl groups were then capped by acetic anhydride activated by N-methyl imidazole in pyridine as a capping reagent. Oxidation step was performed using 0.5% iodine in a mixture of THF:pyridine:H₂O (88:10:2, v/v/v) with an oxidation time of 2×6 min for 18b. The final detritylated oligos were cleaved from the solid support and deprotected at r.t. using conc. NH₄OH (for dC and dZ-containing oligos) or 10% Et₂NH in acetonitrile followed by ethylenediamine/toluene² (for FdZ-containing oligos and eluted from the column with 1 mL milli-Q water). The deprotected oligos in solution were freeze-dried and dry pellets were dissolved in milli-Q water (1 mL) and purified and isolated by reverse phase HPLC on 250/4.6 mm, 5 μm, 300 Å C18 column (Thermo Fisher Scientific) in a gradient of CH₃CN (0→20% for 20 min, 1.3 mL/min) in 0.1 M TEAA buffer (pH 7.0) with a detection at 260 nm. For the deprotection of TIPS protecting group in oligos, the dry pellets were dissolved in a solution of o-nitrophenol (0.050 mmol) in 100 μL THF, followed by 200 μL of 1 M TBAF in THF. The mixture was kept at 22° C. for 30 min. The reaction mixture was quenched by the addition of 2 M TEAA buffer (pH 7.0) and purified by reverse phase HPLC on 250/4.6 mm, 5 μm, 300 Å C18 column (Thermo Fisher Scientific) in a gradient of CH₃CN (0→80% for 14 min, 1.3 mL/min) in 0.1 M TEAA buffer (pH 7.0) with a detection at 260 nm. Oligonucleotides were freeze-dried, pellets were dissolved in milli-Q water (1.5 mL) and desalted by reverse-phase HPLC on 100/10 mm, 5 μm, 300 Å C18 column (Phenomenex) in a gradient of CH₃CN (0→80% for 15 min, 5 mL/min) in milli-Q water with detection at 260 nm. Pure products were quantified by measuring absorbance at 260 nm, analyzed by ESI-MS and concentrated by freeze-drying.

The inventors established that the protocol used for deprotection of FdZ-containing oligos leads to hydration of unprotected triple bonds in dU^{E} and dH^{E1} as determined by ESI-MS showing mass of isolated oligos being 18 units higher than expected (Scheme 6).

To circumvent this problem the inventors synthesized carbazole nucleosides 24b and 25 containing TIPS-protecting group that is stable in DNA synthesis³⁸⁻³⁹ and during cleavage of dZ- as well as FdZ-containing oligos from the CPG support and nucleobase deprotection. However, standard removal of TIPS in FdZ-containing oligos using 1 M tetra-n-butylammonium fluoride (TBAF) in THF³⁹ led to a hydrated oligo showing 18 units higher mass in ESI-MS than calculated. ¹H NMR spectrum of this oligo revealed two strongly coupled doublets at 7.5 and 9.0 ppm, which were previously assigned as three-bond ¹H-¹⁹F coupling in an aldehyde 28 formed after OH⁻ mediated degradation of 5-fluorozebularine (FIG. 2b in reference⁴⁰).

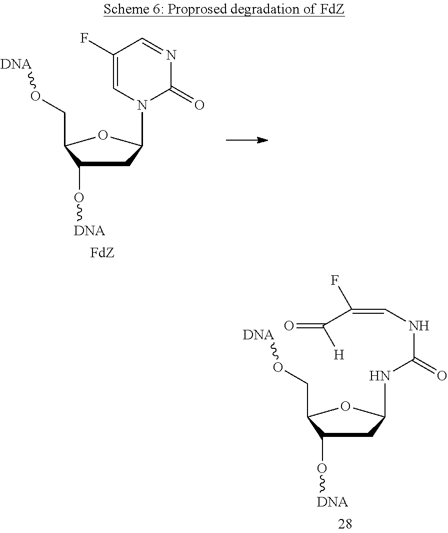

Hydration of FdZ-containing oligos upon treatment with 1 M TBAF in THE forming an aldehyde containing oligo that showed 18 units higher mass in ESI-MS than calculated for the cyclized form of FdZ.

Interestingly, the inventors did not observe hydrated oligos after removal of TIPS for dZ-containing oligos, which is also in line with a previous report that aldehyde formed after zebularine degradation is very reactive and cyclizes back to zebularine.⁴⁰ It also indicates that the terminal triple bond in dH^{E1} is not hydrated under conditions of TIPS-removal.

To prevent OH⁻ mediated degradation of FdZ during TIPS deprotection, the inventors included o-nitrophenol as a weak acid (pKₐ=7.22)⁴¹ to a 1 M TBAF solution in THF, which led to successful cleavage of TIPS and isolation of desired FdZ-containing oligos.

To summarize, dZ-containing oligos can be synthesized using modified nucleotides with unprotected terminal alkynes but deprotection of oligos using conc. NH₄OH should be performed at room temperature. Synthesis of FdZ-containing oligos requires assembly of oligos using nucleotides having TIPS-protected alkynes. After the synthesis, oligos on support are treated by 10% Et₂NH in acetonitrile for 10 min followed by incubation of the support in ethylenediamine/toluene mixture for 2 h at room temperature with subsequent release of TIPS-containing FdZ-oligo in water. Then at this stage or after oligo purification, TIPS-protecting group can be cleaved using 1 M TBAF solution in THF with addition of o-nitrophenol at 22° C. for 30 min.

For the synthesis of oligos containing dH^{E2} and dH^{E3} modifications, inventors used an on-column Sonogashira reaction⁴²⁻⁴⁴ on 3-iodo-carbazole nucleoside 24c after its incorporation into DNA using 1,3-diethynylbenzene or 1,7-octadyne, respectively (Scheme 7). After incorporation of compound 24c on DNA, the support was treated with the mixture containing Pd(PPh₃)₄ (20 mg), CuI (4 mg), Et₃N (300 μL), 1,3-diethynylbenzene (30 μL for dH^{E2}) or 1,7-octadyne (30 μL for dH^{E3}) dissolved in DMF (1 mL). Each solution was freshly prepared and degassed for 30 min. Post-synthetic on-column reaction was performed using semi-automated pump that continuously passed each reaction mixture through the CPG support containing an oligo at 37° C. for 3 h. Afterwards support was washed with DMF (10 mL), ACN (5 mL) and dried. The DNA synthesis was continued until the required sequence was assembled.

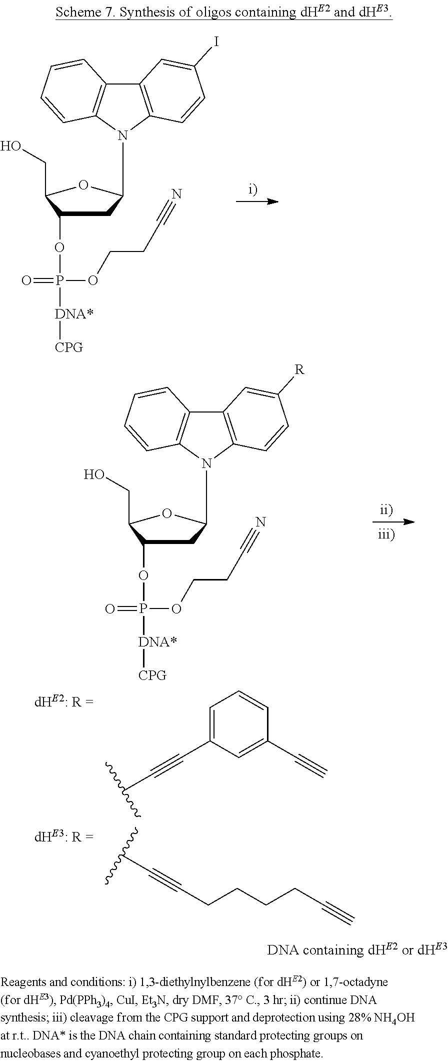

As dH^{E2} and dH^{E3} modifications have a terminal unprotected triple bond and to avoid complications with the hydration of the triple bond, inventors synthesised oligos containing dZ and not FdZ. After the synthesis, these oligos were cleaved from the support and deprotected using concentrated NH₄OH at r.t. The deprotected oligos in solution were freeze-dried and dry pellets were dissolved in milli-Q water (1 mL) and purified and isolated by reverse phase HPLC on 250/4.6 mm, 5 μm, 300 Å C18 column (Thermo Fisher Scientific) in a gradient of CH₃CN (0→20% for 20 min, 1.3 mL/min) in 0.1 M TEAA buffer (pH 7.0) with a detection at 260 nm. Each peak having absorbance at 260 nm was freeze-dried, pellets were dissolved in milli-Q water (1.5 mL) and desalted by reverse-phase HPLC on 100/10 mm, 5 μm, 300 Å C18 column (Phenomenex) in a gradient of CH₃CN (0→80% for 15 min, 5 mL/min) in milli-Q water with detection at 260 nm. Each fraction was analysed by ESI-MS and the fraction containing an oligo with the correct mass was used for the formation of a cross-link using CuAAC.

### 6.4. Example 3: Synthesis of Cross-Linked Oligonucleotides with CuAAC

The purified linear oligonucleotides were cross-linked by copper(I)-catalyzed azide alkyne cycloaddition (CuAAC) using the following protocol.

TABLE 2
Protocol employed for the CuAAC reaction.

| OD₂₆₀ units | 5< | 5-10 | 10-15 | 15-20 | 20-25 | 25-30 | 30-35 | 35-40 |
|---|---|---|---|---|---|---|---|---|
| Oligonucleotide, μL | 30 | 60 | 90 | 120 | 150 | 180 | 210 | 240 |
| 2M TEAA (pH 7.0), μL | 10 | 20 | 30 | 40 | 50 | 60 | 70 | 80 |
| DMSO, μL | 40 | 80 | 120 | 160 | 200 | 240 | 280 | 320 |
| ‘Click catalyst’, μL* | 10 | 20 | 30 | 40 | 50 | 60 | 70 | 80 |
| Sodium ascorbate (10 | 5 | 10 | 15 | 20 | 25 | 30 | 35 | 40 |
| mM), μL | | | | | | | | |

*The ‘click catalyst’ is prepared fresh by mixing 10 mM of tris[(1-benzyl-1H-1,2,3-triazol-4-yl)methyl]amine (TBTA) dissolved in DMSO and 10 mM of Cu(II) sulfate in water (1:1 v/v).

The oligonucleotides (except oligos containing carbazole in the sequence) were dissolved in the required quantity of 2 M TEAA (pH 7.0) followed by addition of DMSO. The bright blue colored ‘click catalyst’ was then added to the above contents in a tube that was purged with argon, followed by addition of freshly prepared sodium ascorbate solution in water. Reaction mixture was kept overnight at room temperature. A 10 μL aliquot of the reaction mixture was taken and the oligonucleotide was precipitated by adding 25 μL of 2 M LiClO₄ and five-fold volume of acetone. The contents were centrifuged using an Eppendorf MiniSpin Plus at 14500 rpm, 14100×g, for 2 min. The supernatant was discarded, and the pellet was washed carefully with acetone and dried. The dry pellet was dissolved in 10 μL milli-Q water and analyzed by reverse-phase HPLC for the product formation. For oligos containing carbazole in the sequence, 10 mM aq. solution of tris(3-hydroxypropyltriazolylmethyl)amine (THPTA) and 10 mM Cu(II) sulfate in water (1:1 v/v) were used instead of TBTA/Cu(II) complex as a ‘click’ catalyst. For short oligos, 10-fold excess of 2 M aq. TEAA was used to keep the solution diluted to prevent formation of dimers and multimers. After the completion of the reaction as shown by reverse phase HPLC, the oligos without carbazole in the sequence were precipitated by adding 1 mL of 2 M aq. LiClO₄ followed by a ten-fold volume of acetone and mixed well. The contents were centrifuged using a Thermo Fisher Heraeus Multifuge X1R centrifuge with swing bucket rotor at 5000 rpm, 4700×g for 30 min. The supernatant was discarded, and the pellet was washed carefully with acetone and dried. The dry pellet was dissolved in 10 mL milli-Q water and the product was isolated by reverse-phase HPLC on 250/4.6 mm, 5 μm, 300 Å C18 column (Thermo Fisher Scientific) in a gradient of CH₃CN (0→20% for 20 min, 1.3 mL/min) in 0.1 M TEAA buffer (pH 7.0) with a detection at 260 nm. Carbazole containing oligos were purified directly after the ‘click’ reaction without further treatments. Short oligos were purified by reverse phase HPLC on 250/4.6 mm, 5 μm, 300 Å C18 column (Thermo Fisher Scientific) in a gradient of CH₃CN (0→80% for 14 min, 1.3 mL/min) in 0.1 M TEAA buffer (pH 7.0) with a detection at 260 nm. Purified oligos were freeze-dried, pellets were dissolved in milli-Q water (1.5 mL) and desalted by reverse-phase HPLC on 100/10 mm, 5 μm, 300 Å C18 column (Phenomenex) in a gradient of CH₃CN (0→80% for 15 min, 5 mL/min) in milli-Q water with detection at 260 nm. Cross-linked oligos were quantified by measuring absorbance at 260 nm, analyzed by ESI-MS and concentrated by freeze-drying.

Progress of the reaction was monitored by reverse-phase HPLC. Gratifyingly, a new peak appeared in the chromatogram for all cross-linking experiments over a period of 16 hr, characterized by a shorter retention time than those of the starting materials (Tables 3 and 4). These results agree with previously published properties of circularized DNA, which were also prepared using CuAAC.²³ After separation by reverse-phase HPLC, the products were desalted and analyzed by ESI-MS showing, as expected, the same mass as for the starting materials, confirming the composition of monomeric cross-linked oligos (Tables 3 and 4). Abbreviation of individual oligos is explained in the caption of Tables 3 and 4.

TABLE 3
List of linear oligos with azide/alkyne modifications as well as terminally
cross-linked oligos having 1,4-disubstituted 1,2,3-triazole modification.

| | | Linear oligos | | Cross-linked (X) oligos | |
|---|---|---|---|---|---|
| Linear DNA | | Retention | | Retention | |
| sequence, 5′→ | Oligo | time | ESI-MS [Da] | time | ESI-MS [Da] |
| 3′ | abbreviation^{a} | (min) | found/calc | (min) | found/calc |
| YTTTCATTdR^{N3} | dC-9mer | 17.5 | 2552.47/ | 14.1 | 2552.47/ |
| | | | 2552.48 | | 2552.48 |
| YTTCATdR^{N3} | dC-7mer | 18.0 | 1944.37/ | 13.6 | 1944.37/ |
| | | | 1944.37 | | 1944.37 |
| YTCAdR^{N3} | dC-5mer | 17.8 | 1336.29/ | 13.5 | 1336.29/ |
| | | | 1336.30 | | 1336.30 |

^{a}dC denotes substrate (dC) of A3 present in position 0 in the DNA sequence. The length of terminally cyclized oligos is described being 9, 7 or 5mer in relation to the length of the native DNA sequence having the same number of phosphate groups. In other words, modifications Y and dR^{N3} are counted as modified nucleotides. X at the end of abbreviation stands for a cross-linked oligo containing 1,4-disubstituted 1,2,3-triazole, i.e. dC-9mer-X refers to a cross-linked oligo.

TABLE 4
List of linear oligos with azide/alkyne modifications as well as internally
cross-linked oligos having 1,4-disubstituted 1,2,3-triazole modification
based on a DNA sequence ATTTCATTT (SEQ ID NO: 1).

| | Linear oligos | | Cross-linked (X) oligos | |
|---|---|---|---|---|
| | Retention | ESI-MS [Da] | Retention | ESI-MS [Da] |
| Oligo | time | found/ | time | found/ |
| abbreviation^{a)} | (min) | calculated | (min) | calculated |

Cross-link 1

| | | | | |
|---|---|---|---|---|
| dC[R^{N3}(−3), A^{Y2}(+1)]^{b)} | 17.51 | 2646.46/2646.48 | 14.69 | 2646.46/2646.48 |
| dC[R^{N3}(−2), A^{Y2}(+1)] | 18.00 | 2646.46/2646.48 | 14.93 | 2646.47/2646.48 |
| dC[R^{N3}(−1), A^{Y2}(+1)] | 17.76 | 2646.46/2646.48 | 14.79 | 2646.47/2646.48 |
| dC[R^{N3}(−2), A^{Y4}(+1)] | 18.02 | 2674.48/2674.51 | 16.03 | 2674.48/2674.51 |
| dZ[R^{N3}(−3), A^{Y2}(+1)] | 17.51 | 2631.44/2631.47 | 14.81 | 2631.45/2631.47 |
| dZ[R^{N3}(−2), A^{Y2}(+1)] | 17.00 | 2631.44/2631.47 | 15.01 | 2631.45/2631.47 |
| dZ[R^{N3}(−1), A^{Y2}(+1)] | 17.76 | 2631.44/2631.47 | 15.02 | 2631.44/2631.47 |
| dZ[R^{N3}(−2), A^{Y4}(+1)] | 18.03 | 2659.47/2659.50 | 16.08 | 2659.47/2659.50 |

Cross-link 2

| | | | | |
|---|---|---|---|---|
| dC[U^{E}(−3), A^{N3}(+1)] | 16.7 | 2757.46/2757.49 | 15.6 | 2757.47/2757.49 |
| dC[U^{E}(−2), A^{N3}(+1)] | 16.3 | 2757.46/2757.49 | 15.3 | 2757.46/2757.49 |
| dZ[U^{E}(−3), A^{N3}(+1)] | 16.9 | 2742.45/2742.48 | 15.7 | 2742.46/2742.48 |
| dZ[U^{E}(−2), A^{N3}(+1)] | 16.9 | 2742.45/2742.48 | 15.7 | 2742.45/2742.48 |

Cross-link 3

| | | | | |
|---|---|---|---|---|
| dC[C^{N3}(−2), H^{E1}(+1)] | 18.08 | 2802.51/2802.54 | 16.52 | 2802.51/2802.54 |
| dZ[C^{N3}(−2), H^{E1}(+1)] | 19.47 | 2787.51/2787.54 | 16.49 | 2787.51/2787.54 |
| FdZ[C^{N3}(−2), H^{E1}(+1)] | 19.63 | 2805.49/2805.52 | 16.52 | 2805.49/2805.50 |
| 4-mer | 11.23 | 1275.27/1275.28 | 9.54 | 1275.27/1275.28 |
| FdZ[C^{N3}(−2), H^{E1}(+1)] | | | | |
| 4-mer dZ[C^{N3}(−2), | 11.20 | 1257.28/1257.29 | 9.59 | 1257.28/1257.29 |
| H^{E1}(+1)] | | | | |
| 3-mer dZ[C^{N3}(−2), | 11.64 | 953.24/953.25 | 9.80 | 953.24/953.25 |
| H^{E1}(+1)] | | | | |
| dZ[C^{N3}(−1), H^{E2}(+1)]* | 13.80 | 2887.54/2887.54 | 11.30 | 2887.54/2887.54 |
| dZ[C^{N3}(−2), H^{E2}(+1)]* | 13.80 | 2887.54/2887.54 | 11.00 | 2887.54/2887.54 |
| dZ[C^{N3}(−1), H^{E3}(+1)]* | 13.40 | 2867.57/2867.57 | 10.70 | 2867.57/2867.57 |
| dZ[C^{N3}(−2), H^{E3}(+1)]* | 13.40 | 2867.57/2867.57 | 10.50 | 2867.57/2867.57 |
| 8-mer FdZ[C^{N3}(−2), | 14.80 | 2501.79/2501.79 | 13.60 | 2501.79/2501.79 |
| H^{E1}(+1)]* | | | | |

^{a)}d stands for a DNA backbone; C, Z or FdZ denote substrate (dC) or inhibitors (dZ, FdZ) of A3 present in position 0 in the DNA sequence ATTTCATTT (SEQ ID NO: 1). The abbreviation of alkyne- and azido-containing nucleotides is presented in square brackets and the position of each modification relative to the position 0 being dC or dZ/FdZ is shown in brackets. X at the end of abbreviation refers to a cross-linked oligo containing 1,4-disubstituted 1,2,3-triazole. For example, abbreviation dC[R^{N3}(−3), A^{Y2}(+1)] indicates a linear cytosine-containing DNA sequence, in which dR^{N3} is present in position −3 instead of T and dA^{Y2} is in position +1 instead of dA of a parent dATTTCATTT (SEQ ID NO: 1) sequence. Letter X at the end of its name, i.e. dC[R^{N3}(−3), A^{Y2}(+1)]X means that it is a cross-linked version of the sequence. Shorter oligos, 8-mer, 5-mer, 4-mer and 3-mer, are based on the shortened parent DNA sequence to 8-mer (dATTTCATT), 5-mer (dTTCAT), 4-mer (dTTCA) and 3-mer (dTCA), respectively, which means that 4-mer FdZ[C^{N3}(−2), H^{E1}(+1)]X is a cross-linked oligo obtained from a linear oligo dC^{N3} T FdZ dH^{E1}.
^{b)}All linear oligos in Table 4 were synthesised using phosphoramidites of azido-containing nucleosides except oligos labelled with an asterisk, which were obtained using H-phosphonates of azido-containing nucleosides.

Additionally, to prove that the cross-linking strategy also works for internal modifications, inventors performed NMR experiments on linear and cross-linked oligos dC[R^{N3}(−2),A^{Y2}(+1)] and dC[IR^{N3}(−2),A^{Y2}(+1)]X, respectively. Differences in chemical shifts between the starting material and the product in ¹H NMR were observed for all methyl peaks of thymidines located between 1.6 to 2.0 ppm suggesting the formation of a new product (cross-linked oligo) as the result of CuAAC reaction. In addition, acetylenic proton at 2.44 ppm in the linear oligo that was assigned by HMBC NMR cross peak with the CH₂ (18.30 ppm in ¹³C NMR) immediately next to it was absent in the cross-linked oligo which provided additional evidence of the successful cross-linking by CuAAC.

It has been reported that A3A and A3B_{CTD} bind to and deaminate cytosine in short 3-4 nucleotide loops of DNA hairpins.¹⁷⁻¹⁹ Inventors synthesized a three-nucleotide loop DNA hairpins as substrates (dC-hairpin-1 and dC-hairpin-2, FIG. 4) and as inhibitors (FdZ-hairpin-1 and FdZ-hairpin-2) and evaluated them together with the cross-linked oligos as described herein.

### 6.5. Example 4: Evaluation of DNA Substrates by NMR-Based Activity Assay

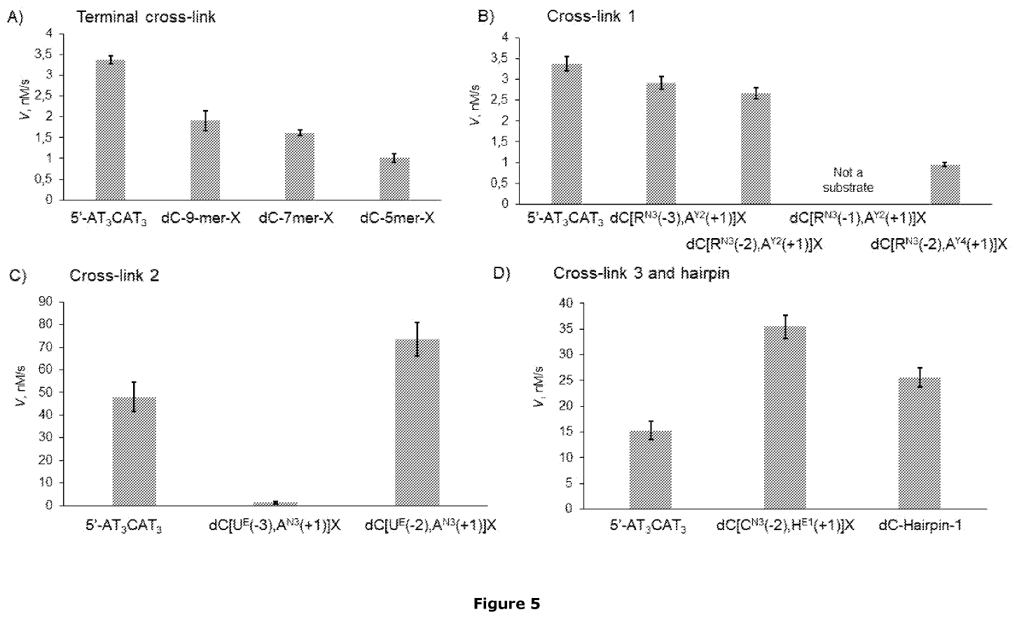

Using the 9-mer oligonucleotide ATTTCATTT (5′-AT3dCAT3) (SEQ ID NO: 1) as the standard substrate, the inventors compared the deamination of both terminally and internally cross-linked oligos prepared as above using a previously described real-time NMR assay.^{1, 45} For the terminally cross-linked oligos, decreased rates of deamination were observed from 9-mer to 5-mer. The number of phosphates of the modified 9-mer sequence is the same as for the 9-mer DNA control (FIG. 5A). A similar decline in deamination rate was observed for oligos having internal cross-link 1 (FIG. 5B). The next internal cross-link 2 was designed based on modified nucleotides dU^{E} and dA^{N3}. In marked contrast, a 1.5 times faster deamination rate was observed for the internally cross-linked oligo dC[U^{E}(−2),A^{N3}(+1)]X compared to the standard, linear substrate (FIG. 5C). An extremely slow deamination rate was detected for dC[U^{E}(−3),A^{N3}(+1)]X oligo. A similar increase in deamination rate to that of internal cross-link 2 was observed for a dC-hairpin-1, which was deaminated 1.6 times faster than the linear substrate (FIG. 5D). However, a marked 2.3 times increase in deamination rate was detected for oligo dC[C^{N3}(−2),H^{E1}(+1)]X having internal cross-link 3 connecting −2 and +1 positions in the DNA sequence (FIG. 5D). It is surprising that synthetic oligo dC[C^{N3}(−2),H^{E1}(+1)]X was deaminated even faster than a native DNA hairpin. Without being bound by theory, the inventors suggest that the high deamination rate may be attributed to additional interactions of carbazole nucleoside in cross-link 3 with amino acid side chains of the enzyme and associated water molecules.

### 6.6. Example 5: Evaluation of Inhibitors by NMR Assay

The inhibition potential of cross-linked oligos was investigated by changing dC to dZ or FdZ in these constructs. The NMR assay described in Example 4 was used to compare the internally cross-linked dZ/FdZ-containing oligos with a linear DNA inhibitor containing dZ/FdZ (dZ-linear is 5′-AT₃dZAT₃, FdZ-linear is 5′-AT₃FdZAT₃) that were characterized earlier.¹⁻² Residual activity of A3B_{CTD}-QM-ΔL3-AL1swap on the unmodified oligo (5′-T4dCAT) as a substrate in the presence of a known concentration of dZ/FdZ-containing inhibitors (linear and cross-linked) was measured using NMR assay.

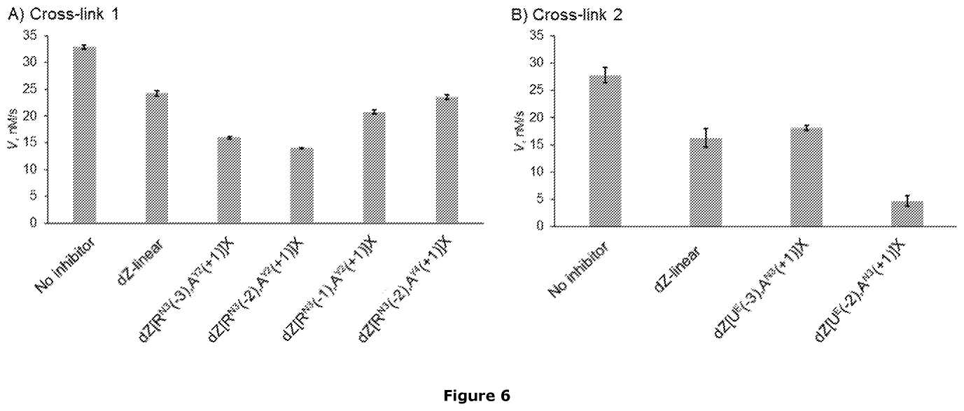

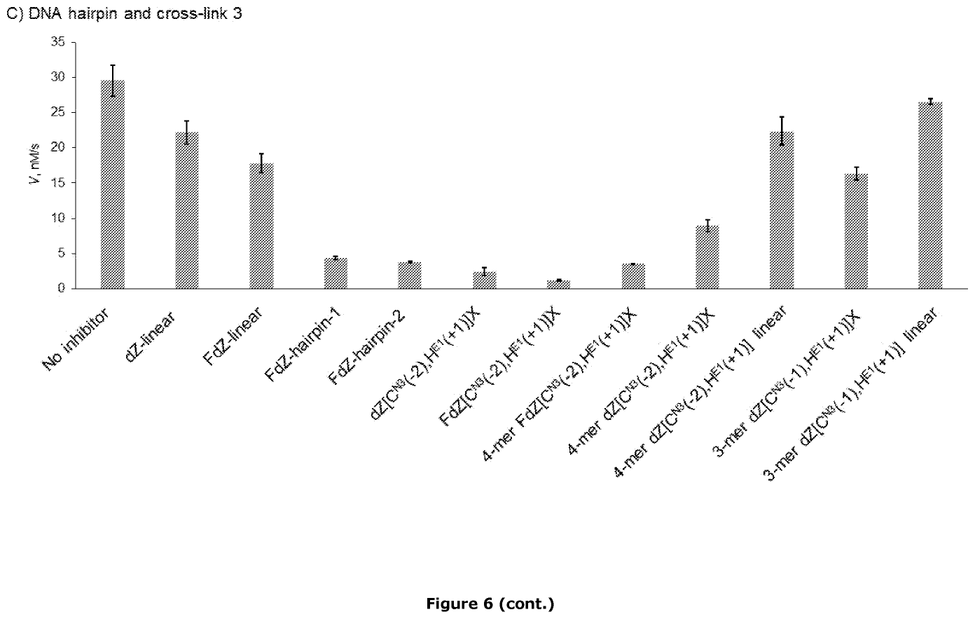

The results revealed that the dZ-containing oligo with an internal cross-link 2 derived from the faster-deaminated substrate dZ[U^{E}(−2),A^{N3}(+1)]X inhibited the A3-catalyzed deamination by greater than an order of magnitude more potently than the best linear dZ oligo (FIG. 6B). On the other hand, dZ[U^{E}(−3),A^{N3}(+1)]X was found to be a weaker inhibitor.

The fastest-deaminated substrate dC[C^{N3}(−2),H^{E1}(+1)]X based on internal cross-link 3 was converted to the most powerful A3 inhibitors studied here having dZ or FdZ nucleotides instead of dC (FIG. 6C). It is notable that FdZ-containing oligo FdZ[C^{N3}(−2),H^{E1}(+1)]X exhibited higher inhibitory potential than dZ-containing oligo dZ[C^{N3}(−2),H^{E1}(+1)]X, which is in line with the inventors' previous observations² that FdZ-linear oligo is a more powerful inhibitor of A3B_{CTD}-QM-ΔL3-AL1swap than dZ-linear oligo (FIG. 6C). It is also interesting that FdZ-hairpin-1 DNA was not as a powerful an inhibitor as cross-linked oligo FdZ[C^{N3}(−2),H^{E1}(+1)]X, which also correlates with lower deamination rate for dC-hairpin-1 in comparison with dC[C^{N3}(−2),H^{E1}(+1)]X (FIG. 5D).

Short 3-mer and 4-mer oligo inhibitors were also synthesized, based on cross-link 3 that contained dZ or FdZ. The 4-mer FdZ[C^{N3}(−2),H^{E1}(+1)]X was a less potent inhibitor than the cross-linked 9-mer oligo but its inhibitory potential was close to the FdZ-hairpin.

Further decline in inhibition of A3B_{CTD}-QM-ΔL3-AL1swap catalyzed C-to-U deamination was observed upon shortening of the DNA length to the 3-mer (−1,+1 link) and for the non-cross-linked oligos (linear oligos containing modified nucleotides before cross-linking).

For determination of inhibition constants (K_{I}) of inhibitors the inventors first evaluated kinetic parameters of A3B_{CTD}-QM-ΔL3-AL1swap on two DNA substrates (Table 5), which were used to detect the residual deamination rate in the presence of inhibitors (Table 6). Fitting the experimental data by non-linear least squares using a global fit provided similar values of kinetic parameters to those obtained by the linearized Lineweaver-Burk plot (Table 5). Then, the inhibition constants for strongest cross-linked inhibitors were calculated by several methods assuming a competitive mode of inhibition¹ (Table 6) based on experiments in which the concentration of the individual inhibitor was varied, and the residual deamination initial rate was measured.

TABLE 5
Kinetic parameters of A3B_{CTD}-QM-ΔL3-AL1swap
on two DNA substrates used in this work

| | | Linear analysis | Non-linear |
|---|---|---|---|
| | | (Lineweaver- | regression |
| Oligo | Parameters | Burk plot) | analysis |
| 5′-AT₃dCAT₃ | Kₘ, μM | 200 ± 30 | 150 ± 30 |
| | Vₘₐₓ, μM/s | 0.014 ± 0.002 | 0.0126 ± 0.0008 |
| | k_{cat}, s⁻¹ | 0.29 ± 0.04 | 0.26 ± 0.02 |
| | k_{cat}/Kₘ, s⁻¹ μM⁻¹ | 0.0014 | 0.0018 |
| 5′-T₄dCAT | Kₘ, μM | 350 ± 70 | 290 ± 70 |
| | Vₘₐₓ, μM/s | 0.056 ± 0.008 | 0.051 ± 0.005 |
| | k_{cat}, s⁻¹ | 0.19 ± 0.03 | 0.171 ± 0.015 |
| | k_{cat}/Kₘ, s⁻¹ μM⁻¹ | 0.0005 | 0.0006 |

TABLE 6
The inhibition constants (Kᵢ) for dZ/FdZ-containing oligo inhibitors
of A3B_{CTD}-QM-ΔL3-AL1swap obtained by various calculation methods^{a)}

| | | Lineweaver-Burk | |
|---|---|---|---|
| | | plot for the | Non-linear regression |
| | | substrate/Dixon | analysis for both |
| Oligo name | Parameters | plot for the inhibitor | substrate and inhibitor |
| dZ-linear | Kᵢ, μM | 7.5 ± 1.7 | 7.0 ± 1.3 |
| | Vₘₐₓ, μM/s | | 0.0126 ± 0.0008 |
| FdZ-linear | Kᵢ, μM | 2.1 ± 0.8 | 4.4 ± 0.7 |
| | Vₘₐₓ, μM/s | | 0.0126 ± 0.0008 |
| dZ[U^{E}(−2), | Kᵢ, μM | 0.66 ± 0.14 | 0.69 ± 0.14 |
| A^{N3}(+1)]X | | | |
| | Vₘₐₓ, μM/s | | 0.052 ± 0.006 |
| FdZ-hairpin-1 | Kᵢ, μM | 0.28 ± 0.05 | 0.35 ± 0.06 |
| | Vₘₐₓ, μM/s | | 0.052 ± 0.005 |
| FdZ[C^{N3}(−2), | Kᵢ, μM | 0.075 ± 0.015 | 0.100 ± 0.016 |
| H^{E1}(+1)]X | | | |
| | Vₘₐₓ, μM/s | | 0.051 ± 0.004 |
| 4-mer FdZ[C^{N3}(−2), | Kᵢ, μM | 0.25 ± 0.05 | 0.28 ± 0.05 |
| H^{E1}(+1)]X | | | |
| | Vₘₐₓ, μM/s | | 0.051 ± 0.005 |

^{a)}dZ/FdZ-linear inhibitors were evaluated against 5′-AT₃dCAT₃substrate using 50 nM of A3B_{CTD}-QM-ΔL3-AL1swap; all other inhibitors were evaluated against 5′-T₄dCAT substrate using 200 nM of A3B_{CTD}-QM-ΔL3-AL1swap, see caption of FIG. 5 for buffer conditions.

All calculation methods revealed that cross-linked and hairpin dZ/FdZ oligos are more powerful inhibitors of A3B_{CTD}-QM-ΔL3-AL1swap than linear oligos. The Kᵢ values calculated for FdZ[C^{N3}(−2),H^{E1}(+1)]X were lowest among inhibitors and Kᵢ of 0.100±0.016 μM obtained using non-linear regression analysis is around 44 times lower than that for the linear FdZ-oligo (4.4±0.7 μM).² The nanomolar inhibitory potential exhibited by the internally cross-linked oligos shows that properly pre-shaped, cross-linked DNA can become a better inhibitor than the linear one and that the better substrate becomes the better inhibitor, which supports the original hypothesis of inventors.

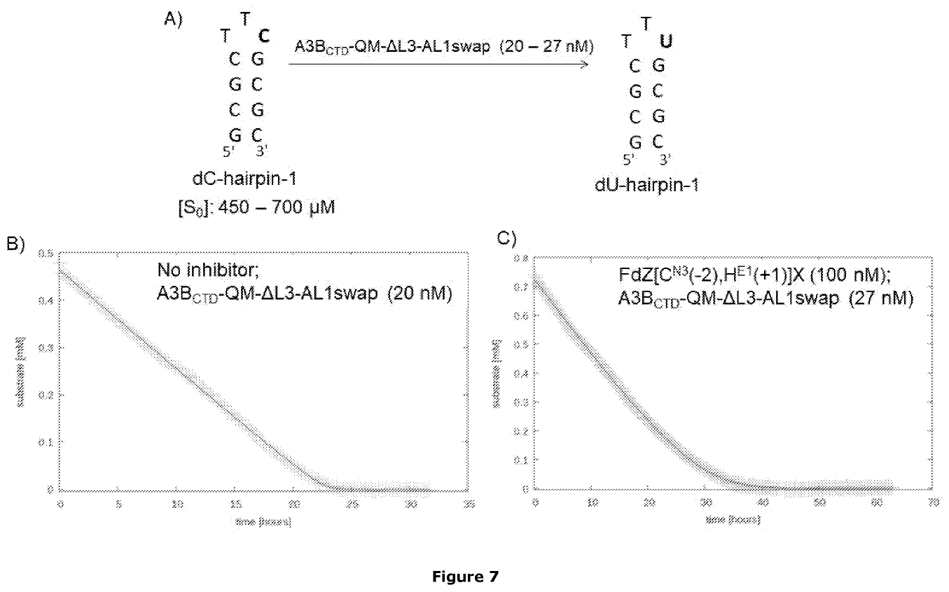

To further characterize the best inhibitor FdZ[C^{N3}(−2),H^{E1}(+1)]X inventors performed deamination of dC-hairpin-1 (FIG. 4), which is a faster deaminating substrate than linear DNA (FIG. 5D), using a much lower concentration of enzyme than in previous assays (20-27 nM versus 200 nM) in the absence and presence of inhibitor (100 nM). The kinetic data, measured over time until exhaustion of the substrate (FIG. 7), were analyzed by global regression using Lambert's W function, which provides superior estimates of observed Kₘ and Vₘₐₓ than non-linear regression analysis of initial rate data or any of the known linearized transformations of the Michaelis-Menten equation, such as Lineweaver-Burk, Hanes-Woolf and the Eadie-Hofstee transformations.⁴⁶

TABLE 7
Kinetic parameters of A3B_{CTD}-QM-ΔL3-AL1swap-catalyzed
deamination of dC-hairpin in the absence and presence of FdZ[C^{N3}(−2),
H^{E1}(+1)]X obtained using Lambert's W function

| | | | Kₘ | Kᵢ | k_{cat}/ |
|---|---|---|---|---|---|
| | Vₘₐₓ, | k_{cat}, | (observed), | (calculated), | Kₘ, s⁻¹ |
| Inhibitor | nM/s | s⁻¹ | μM | nM | μM⁻¹ |
| No inhibitor | 8.0 ± 0.4 | 0.296 ± 0.015 | 6.6 ± 0.7 | — | 0.045 |
| FdZ[C^{N3}(−2), | 8.4 ± 0.4 | 0.311 ± 0.015 | 104 ± 5 | 6.8 ± 1.4 | — |
| H^{E1}(+1)]X (100 | | | | | |
| nM) | | | | | |

Under given experimental conditions, it is evident that dC-hairpin-1 is a better substrate of A3B_{CTD}-QM-ΔL3-AL1swap than linear oligos 5′-AT₃dCAT3 and 5′-T₄dCAT because its Kₘ is in low μM range and k_{cat} of enzyme is similar on these substrates. The specificity constant defined by k_{cat}/Kₘ reinforces that the A3B_{CTD}-QM-ΔL3-AL1swap is ˜25 times more efficient at converting the dC-hairpin-1 than the linear 5′-AT3dCAT₃. In the presence of inhibitor (100 nM), Vₘₐₓ was unchanged (Table 7) which indicates that FdZ[C^{N3}(−2),H^{E1}(+1)]X is a competitive inhibitor of A3B_{CTD}. The calculated inhibition constant (Kᵢ) from the observed Kₘ values in the absence and in the presence of inhibitor was found to be in the low nM range (Kᵢ=6.8±1.4 nM), which again shows that FdZ[C^{N3}(−2),H^{E1}(+1)]X is a powerful inhibitor of A3B_{CTD}.

### 6.7. Example 6: Binding of Cross-Linked Oligos to A3 Enzymes

The inventors used isothermal titration calorimetry (ITC) to determine binding of the cross-linked oligos to A3 enzymes, specifically to A3A-E72A, where the active-site glutamic acid is replaced with alanine, and to the catalytically competent A3B_{CTD}-QM-ΔL3-AL1swap and compared this binding to that of linear oligos.

Desalted unmodified DNA oligonucleotides were purchased (Integrated DNA Technologies) at 1 or 5 μmol synthesis scale and dissolved in one of the buffers described below to give 10 mM solutions. ITC experiments were conducted at 25° C. using a MicroCal ITC200 (now Malvern Instruments) isothermal titration calorimeter. Protein A3A-E72A, which is an inactive protein, was diluted in ITC buffer to concentrations of 10-100 μM and titrated with dC oligonucleotides [5′-AT3dCAT3, 5′-T₄dCAT, cross-linked oligos] in ITC buffer. The ratio of protein to oligonucleotide concentration is usually 1:10. Titrations are made up of 30 individual additions. To prevent protein precipitation during the long time-scale of the experiment it was necessary to used improved ITC buffers. ITC buffer 1: 50 mM MES, pH 6.0, 100 mM NaCl, 200 μM EDTA, 1 mM β-mercaptoethanol and ITC buffer 2: 50 mM Na⁺/K⁺ phosphate, pH 6.0, 50 mM NaCl, 50 mM choline acetate, 2.5 mM TCEP, 200 μM EDTA with 30 mg/mL BSA. After preparation, ITC buffer 2 was frozen and defrosted before the experiments. ITC buffer 2* is freshly prepared buffer 2.

For oligos containing dZ, catalytically active A3B_{CTD}-QM-ΔL3-AL1swap was used, as it has the active-site glutamic acid required for protonation of N₃ in dZ, which activates C₄ in dZ to accept nucleophilic OH⁻/H₂O coordinated to the Zn²⁺, which converts dZ into a transition-state or an intermediate analogue of cytidine deamination.⁸

For A3B_{CTD}-QM-ΔL3-AL1swap we used ITC buffer 3: 50 mM Na⁺/K⁺ phosphate, pH 6.0, 200 mM trimethylamine N-oxide dihydrate, 2.5 mM TCEP, 200 μM EDTA. Data evaluation was performed with the software provided by the supplier of MicroCal ITC200.

In contrast, A3A-E72 Å was used, particularly for oligos containing dC so that the complications of deamination can be avoided, although this mutation, notwithstanding no change to active-site structure, does cause a small but significant diminution in ligand binding¹ through loss of hydrogen bonding to the ligand. The inventors expect that the binding of inactivated A3B_{CTD}-QM-ΔL3-AL1swap to the substrate will be close to or even worse than Kₘ of 200 μM, which places substrate binding outside ITC applicability. Results of ITC experiments are presented in Table 8. One should emphasise that the composition of buffers used for NMR kinetic and ITC binding experiments are different, mainly because proteins in ITC experiments need to be at much higher concentration than for kinetic assays (10-100 μM for ITC versus 20-300 nM in kinetic experiments).

This required a search for buffer components that prevent protein precipitation during ITC titrations. The optimized ITC buffers were also different for A3A(E72A) and A3B_{CTD}-QM-ΔL3-AL1swap, which means that K_{d} and Kᵢ values cannot be directly compared.

TABLE 8
Dissociation constant of oligos from A3 enzymes obtained
by isothermal titration calorimetry (ITC).^{a}

| | | ITC | |
|---|---|---|---|
| Oligo | Protein | buffer^{b} | K_{d} (μM) |
| 5′-AT₃dCAT₃ | A3A-E72A | 1 | 0.20 ± 0.06 |
| 5′-T₄dCAT | A3A-E72A | 1 | 0.19 ± 0.04 |
| dC-9mer-X | A3A-E72A | 1 | 0.26 ± 0.06 |
| dC-7mer-X | A3A-E72A | 1 | 0.25 ± 0.06 |
| dC-5mer-X | A3A-E72A | 1 | 2.7 ± 0.6 |
| Cross-link 1 | A3A-E72A | 1 | 0.28 ± 0.06 |
| dC[R^{N3}(−2), A^{Y2}(+1)] | | | |
| Cross-link 1 | A3A-E72A | 1 | 0.15 ± 0.06 |
| dC[R^{N3}(−2),A^{Y4}(+1)] | | | |
| 5′-AT₃dCAT₃ | A3A-E72A | 2 | 0.12 ± 0.03 |
| dC-hairpin-1 | A3A-E72A | 2 | 0.11 ± 0.03 |
| Cross-link 1 | A3A-E72A | 2 | 0.13 ± 0.04 |
| dC[R^{N3}(−2), A^{Y2}(+1)] | | | |
| Cross-link 1 | A3A-E72A | 2 | 0.15 ± 0.03 |
| dC[R^{N3}(−2), A^{Y2}(+1)] | | | |
| Cross-link 3 | A3A-E72A | 2 | 0.028 ± 0.006 |
| dC[C^{N3}(−2), H^{E1}(+1) ]X | | | |
| 5′-AT₃dCAT₃ | A3A-E72A | 2* | 0.13 ± 0.03 |
| Cross-link 2 | A3A-E72A | 2* | 0.027 ± 0.006 |
| dC[U^{E}(−2), A^{N3}(+1)]X | | | |
| dZ-linear | A3B_{CTD}-QM- | 3 | 11.4 ± 2.3 |
| | ΔL3- | | |
| | AL1swap | | |
| Cross-link 2 | A3B_{CTD}-QM- | 3 | 2.5 ± 0.5 |
| dZ[U^{E}(−2), A^{N3}(+1)]X | ΔL3- | | |
| | AL1swap | | |

^{a}K_{d} is the dissociation constant for the ligand from A3.
^{b}ITC buffer 1: 50 mM MES, pH 6.0, 100 mM NaCl, 200 μM EDTA, 1 mM β-mercaptoethanol; ITC buffer 2: 50 mM Na⁺/K⁺ phosphate, pH 6.0, 50 mM NaCl, 50 mM choline acetate, 2.5 mM TCEP, 200 μM EDTA with 30 mg/mL bovine serum albumin (after preparation, buffer was frozen and defrosted before the experiments); ITC buffer 2* is freshly prepared buffer 2; ITC buffer 3: 50 mM Na⁺/K⁺ phosphate, pH 6.0, 200 mM trimethylamine N-oxide dihydrate, 2.5 mM tris(2-carboxyethyl)phosphine (TCEP), 200 μM EDTA.

For oligos with internal cross-link 1 a slightly higher affinity was detected for the oligo with four-carbon linker dC[R^{N3}(−2),A^{Y4}(+1)] in buffer 1 but not in buffer 2. It is worth mentioning that the substrates with cross-link 1 were not deaminated faster than the linear substrate by A3B_{CTD}-QM-ΔL3-AL1swap (FIG. 5A), which is confirmed by a similar affinity of oligos with cross-link 1 in ITC.

The dC-hairpin-1 and linear oligo 5′-AT3dCAT3 have similar affinity to A3A(E72A) in buffer 2 (pH 6.0). It has been reported that hairpins are preferred substrates for wild-type A3A at pH 7.0 indicating that buffer pH might have a prevalent role in discrimination between linear and hairpin DNA.

dC-Oligo with internal cross-link 3 showed 4-times improved affinity to A3A(E72A) in comparison with linear DNA. This agrees with the improved deamination of this oligo by A3B_{CTD}-QM-ΔL3-AL1swap.

The inventors noticed that the use of freshly prepared buffer 2 (marked as 2* in the Table 8) led to significantly improved stability of A3A(E72A) in solution. It did not affect the affinity of the linear oligo 5′-AT3dCAT3 to A3A(E72A) as K_{d} values in buffers 2 and 2* are similar (K_{d}=0.12±0.03 and 0.13±0.03 μM, respectively). The oligo dC[U^{E}(−2),A^{N3}(+1)]X with internal cross-link 2, which gave high substrate activity, has bound to A3A(72A) four times more strongly (K_{d}=0.027±0.006 μM) in comparison with the linear substrate oligo in buffer 2* (5′-AT3dCAT3, K_{d}=0.13±0.03 μM). The higher substrate activity of this oligo to A3B_{CTD}-QM-ΔL3-AL1swap is backed by the increased binding affinity as shown by our ITC experiments.

Next, inventors evaluated the binding data of dZ-containing oligos towards the catalytically competent A3B_{CTD}-QM-ΔL3-AL1swap. The binding constant of inhibitor dZ[U^{E}(−2),A^{N3}(+1)]X (K_{d}=2.5±0.5 μM) was around 4.5 times more favourable than that of dZ-containing linear oligo (K_{d}=11.4±2.3 μM), similar to the difference seen in substrates. This trend is also consistent with the trend observed in inhibition constants (Kᵢ) determined by the inventors' real-time NMR assay. This effect is smaller in ITC measurements, most likely due to the differences in experimental conditions, as the ITC experiments were conducted in a different buffer and in the presence of trimethylamine N-oxide (TMAO) to stabilize the enzyme.

### 6.8. Example 7: Crystal Structure of DNA Hairpins in Complex with APOBEC3A(E72A)

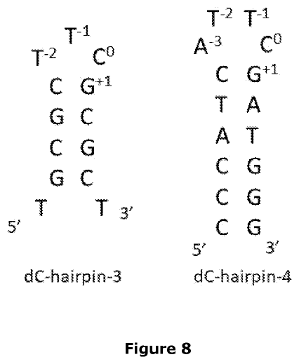

The inventors established that internal cross-links 2 and 3 and dC-hairpin-1 DNA are deaminated faster than linear ssDNA (FIGS. 5C and D). To understand the structural features responsible for this, the inventors crystallised APOBEC3A(E72A) protein with dC-hairpin-3 and dC-hairpin-4 (FIG. 8). Data collection and processing statistics as well as structure refinement statistics are provided in Table 9.

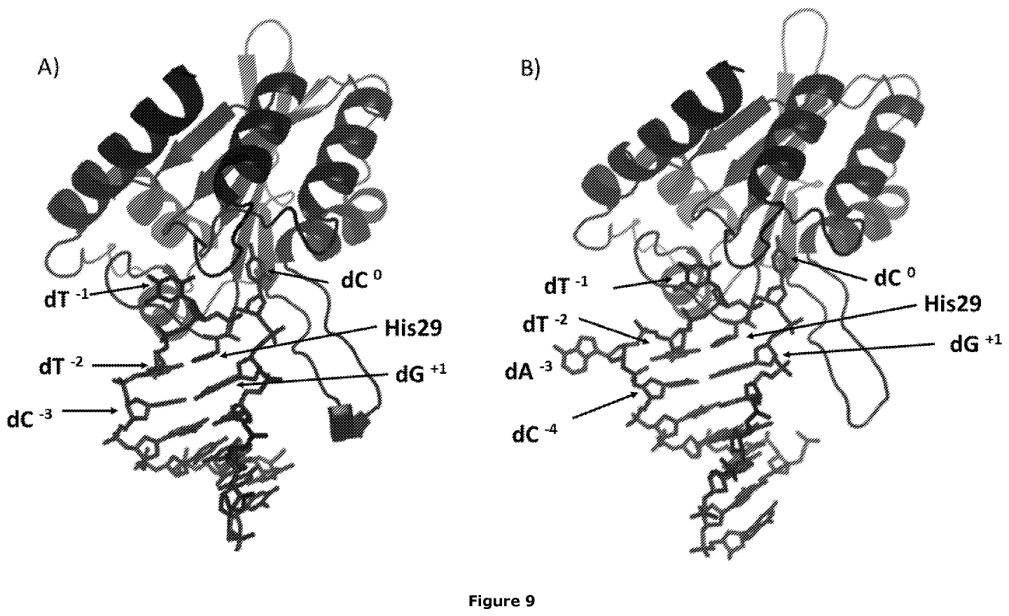

In both structures protein and hairpin components are well-defined (FIG. 9). The thymidine pair at the end of hairpins of one molecule base-stacks with the thymidine pair of the other molecule of the asymmetric unit. The packing of protein and DNA is very tight, but unlike the ssDNA structures, protein residues of one subunit do not interact with the hairpin DNA of another subunit.

Directionality, conformation and nucleotide-protein interactions of dC⁰ and dT⁻¹ are the same in these structures as in the structure of linear ssDNA with APOBEC3A(E72A), pdb numbers 5 keg and 5td5.⁴⁷ dG⁺¹ occupy the same space as dA⁺¹ in 5td5 and dT⁺¹ in 5 keg but the dG⁺¹ nucleobase is flipped to form a H-bonding interaction with the complementary dC⁻³ in dC-hairpin-2 and dC⁻⁴ in dC-hairpin-3 (FIG. 9).

Without being bound by theory, the inventors believe that the most important interaction that makes a hairpin the preferred substrate and better inhibitor if dC is replaced with dZ/FdZ in comparison with the linear ssDNA, is the fact that histidine 29 (His29) of APOBEC3A is in the same plane with thymine −2 which stack with guanine (+1)/cytosine(−3 in dC-hairpin-3 or −4 in dC-hairpin −4) base-pair of the hairpin stem. The thymine nucleobase at position −2 also makes a cation-n interaction with the guanidinium group of Arg28. All these interactions result in an involvement of His29 of the protein in extended base-stacking in the stem of both hairpins dC-hairpin-3 and dC-hairpin-4. Lysine in position 60 of the protein (Lys60) also forms a H-bond with the phosphate present between dG⁺¹ and dC⁺² of hairpins.

TABLE 9
Crystal data, data collection and structure refinement details
for APOBEC3A(E72A) with dC-hairpin-3 and dC-hairpin-4

| | APOBEC3A-E72A/ | | APOBEC3A-E72A/ | |
|---|---|---|---|---|
| Protein/DNA | dC-hairpin-3 ^{a} | | dC-hairpin-4 | |
| Ligand | 5′-T(GC)₂TTC(GC)₂T | | 5′-C₃ATCATTCGATG₃ | |
| Crystal data | | | | |
| Beamline (Temperature) | MX2 (100 K) #131 | | MX2 (100 K) #84 | |
| dataset # | | | | |
| a (/Å) | 53.92 | | 55.00 | |
| b (/Å) | 56.90 | | 57.23 | |
| c (/Å) | 91.55 | | 92.75 | |
| β (/°) | 103.6 | | 106.5 | |
| Crystal habit | Flattened needle | | Flattened needle | |
| Crystal system | Monoclinic | | Monoclinic | |
| Space group | P2₁ | | P2₁ | |
| Z′ | 2 | | 2 | |
| Data collection | | | | |
| Resolution [highest] | 47.94-2.22 | [2.29-2.22] | 48.13-1.91 | [1.96-1.91] |
| # reflections | 89 061 | [6814] | 142 158 | [8859] |
| # unique reflns | 26 243 | [2098] | 42 309 | [2706] |
| # reflns for R_{free} | 1309 | | 2103 | |
| Redundancy | 3.4 | [3.2] | 3.4 | [3.3] |
| Completeness | 97.3 | [85.1] | 98.4 | [94.2] |
| R_{merge} | 0.092 | [1.535] | 0.090 | [0.913] |
| Rₚᵢₘ | 0.059 | [0.979] | 0.057 | [0.589] |
| CC_{1/2} | 0.995 | [0.332] | 0.996 | [0.471] |
| I/σ(I) | 6.2 | [0.6] | 7.5 | [1.0] |
| Mosaicity (/°) | 0.37 | | 0.08 | |
| Wilson B (/Å²) | 51.0 | | 26.3 | |
| Structure refinement | | | | |
| Resolution [highest] | 47.94-2.22 | [2.28-2.22] | 48.17-1.91 | [1.96-1.91] |
| R_{work} | 0.233 | [0.369] | 0.201 | [0.0.308] |
| R_{free} | 0.274 | [0.411] | 0.233 | [0.302] |
| # atoms (total) | 3616 | | 3915 | |
| # protein atoms | 3045 | | 3098 | |
| # nucleotide atoms | 520 | | 614 | |
| # other atoms | 2 × I6P, 2 × (½) Zn²⁺, 2 × Cl⁻ | | 1 × PO₄, 1 × I6P, 2 × Zn²⁺, 2 × Cl⁻ | |
| # water molecules | 11 | | 147 | |
| Wilson B (/Å²) | 57.2 | | 26.3 | |
| Rmsd bond distances (/Å) | 0.006 | | 0.010 | |
| Rmsd bond angles (/°) | 1.58 | | 1.90 | |
| Ramachandran (COOT) | 91.9/6.5/1.6 | | 94.8/5.2/0.0 | |
| OK/OK-ish/Bad (/%) | | | | |

^{a} For refinement, data were truncated at 2.10 Å, giving a working set of 30 741 [2278] reflections and R_{free} set of 1622 [114] reflections, with improved completeness in the highest resolution shell of 98.9%.

The most notable difference between structures with hairpins is bulging of dA⁻³ in dC-hairpin-4 that is essential for the stacking between dT⁻²/His29 pair and G-C nucleotides in the stem to occur (FIG. 9). It also means that other nucleotides can be present instead of dA⁻³ in the sequence of dC-hairpin-2 to allow for efficient deamination. The number of nucleotides in the bulge can be extended to two or even three, providing that such a bulge minimally interferes with interactions between dT⁻²/His29 pair and nucleotide base-pairs in the stem. The inventors argue that nucleotides in the loop of a hairpin can be native or chemically modified nucleotides and if these modified nucleotides at least preserve DNA-protein interactions observed in these crystal structures the modified hairpin will bind to the protein.

From these crystal structures and the inventors' inhibition data of FdZ-hairpin-1 and FdZ-hairpin-2 one can conclude that the composition of nucleotides in the stem can vary but it is essential for the stem to be formed to provide a nucleotide base-pair similar to dG⁺¹/dC⁻³ (in dC-hairpin-3), dG⁺¹/dC⁻⁴ (in dC-hairpin-4) and dA⁺¹/dT⁻³ (in FdZ-hairpin-2), that can stack with the dT⁻²/His29 pair. This means that at least three nucleotide base-pairs need to be present in the sequence of single-stranded DNA to form a thermodynamically stable stem and that the stem can be further extended to 10 base-pairs forming a more thermodynamically stable hairpin.

Several groups in the past evaluated pyrimidinone nucleotides instead of native nucleotides in the middle of palindromic DNA sequences which were self-complementary and meant to form duplexes in solution.⁴⁸⁻⁵¹ Thermal stability of these oligos in solution was evaluated and in one case⁵¹ it was noted that the sequence most likely existed as a mixture of a duplex and a hairpin because chemical modification destabilised a duplex. Inventors identified that formation of a DNA hairpin in solution is essential for binding and inhibition of APOBEC3A and APOBEC3B. Inventors surprisingly observed that the length of hairpin loop can vary and that nucleotides as dA⁻³ in dC-hairpin-4 can be bulged out and not interfere with the interactions of the rest of the hairpin with the protein.

One or more nucleotide derivatives can be included in a hairpin to create a compound of the invention with equivalent or even enhanced properties. As would be understood by a person skilled in the art, the particular derivatives used and their placement within the hairpin would need to selected so as not to disrupt the 3D structure formed by nucleotides in −2, −1, and 0 positions with secondary perturbations caused by moieties in −3 and +1 positions, as observed in the crystal structures generated by the inventors. Accordingly, nucleotide derivatives that would be acceptable in one position in the oligo sequence, might not necessarily be acceptable in another position. For example, RNA-like molecules such as LNA, 2′-OMe-RNA, 2′-F-RNA could be used in the hairpin stem to increase its thermal stability but might diminish binding to A3 if present in −2 and/or −1 and/or 0 positions, as these positions require C(2′)-endo (south) conformation, a DNA-like conformation, of the sugar ring for proper interaction with A3.

### 6.9. Example 8: Evaluation of CDA Inhibitors in the Structure of DNA as Inhibitors of APOBEC3 Enzymes

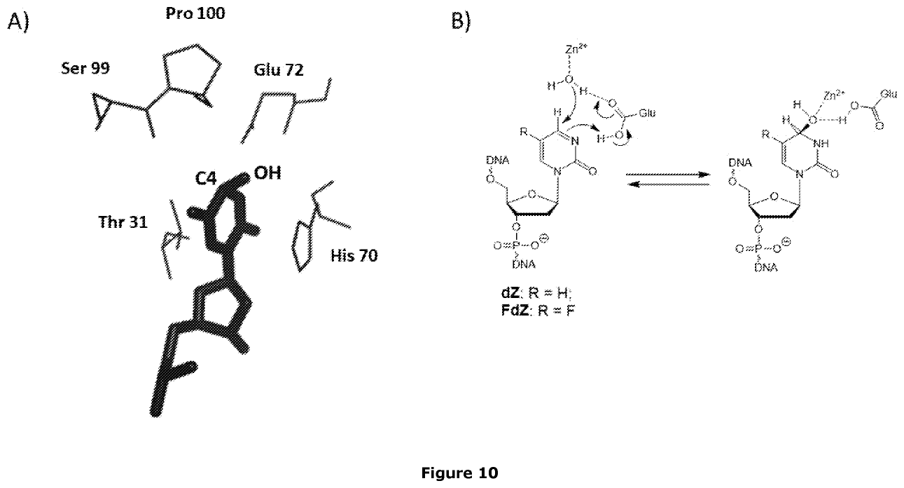

To elucidate structural features of FdZ-containing oligos bound to the active APOBEC3 enzymes, inventors crystallised FdZ-hairpin-1 in a complex with wild-type APOBEC3A. Data collection and processing statistics as well as structure refinement statistics for two datasets are provided in Table 10. Overall structure of FdZ-hairpin-1 bound to APOBEC3A is similar to the structure of dC-hairpin-1 complexed with APOBEC3A(E72A) (FIG. 9A). The major difference was observed for FdZ which was converted into its hydrated form across N3-C4 bond by the enzyme (FIG. 10). The resolution and quality of the crystal data was sufficient to conclude that carbon C4 of FdZ was bound to OH and that its geometry was tetrahedral and not trigonal planar. The 4-OH group was clearly also a fourth ligand of Zn²⁺. Mechanistic pathway for this transformation is illustrated in FIG. 10B. The structure of hydrated FdZ bound to APOBEC3A is similar to the structure of hydrated 5-fluorozebularine, namely 5-fluoro-3,4-dihydrouridine, bound to E. coli cytidine deaminase (CDA).⁶ The similarity between fluorozebularines bound to CDA and APOBEC3A supports inventors' argument that any nucleoside-like inhibitor of CDA can be converted into APOBEC3 inhibitor upon its incorporation into DNA. To further support this argument inventors synthesised following compounds based on seven-membered ring nucleobases.¹⁰

TABLE 10
Crystal data, data collection and structure refinement details
for two structures of wild-type APOBEC3A with FdZ-hairpin-1.

| Protein | Structure 1 | | Structure 2 | |
|---|---|---|---|---|
| Crystal data | | | | |
| Beamline (Temperature) | MX2 (100 K) #339 | | MX2 (100 K) #106 + #108 | |
| dataset # | | | | |
| a (/Å) | 49.94 | | 53.32 | |
| b (/Å) | 56.37 | | 57.20 | |
| c (/Å) | 90.11 | | 91.42 | |
| β (/°) | 104.00 | | 99.37 | |
| Crystal habit | Flattened needle | | Flattened needle | |
| Crystal system | Monoclinic | | Monoclinic | |
| Space group | P2₁ | | P2₁ | |
| Z′ | 2 | | 2 | |
| Data collection | | | | |
| Resolution [highest] | 48.24-2.80 | [2.95-2.80] | 49.05-2.94 | [3.12-2.94] |
| # reflections | 39 433 | [5461] | 45 891 | [7086] |
| # unique reflns | 11 861 | [1629] | 11 619 | [1817] |
| # reflns for R_{free} | 623 | | 567 | |
| Redundancy | 3.3 | [3.4] | 3.9 | [3.9] |
| Completeness | 97.8 | [92.8] | 99.0 | [97.1] |
| R_{merge} | 0.183 | [1.157] | 0.166 | [0.583] |
| Rₚᵢₘ | 0.118 | [0.735] | 0.094 | [0.338] |
| CC_{1/2} | 0.979 | [0.466] | 0.985 | [0.918] |
| I/σ(I) | 4.5 | [0.8] | 5.8 | [2.2] |
| Mosaicity (/°) | 0.28 | | 0.33 | |
| Wilson B (/Å2) | 57.0 | | 42.3 | |
| Structure refinement | | | | |
| Resolution [highest] | 48.29-2.80 | [2.87-2.80] | 48.35 [2.94] | [3.02-2.94] |
| R_{work} | 0.260 | [0.364] | 0.252 | [0.385] |
| R_{free} | 0.323 | [0.370] | 0.314 | [0.356] |
| # atoms (total) | 3325 | | 3368 | |
| # protein atoms | 2926 | | 2884 | |
| # nucleotide atoms | 164 + 202 | | 224 + 224 | |
| # other atoms | 6 × PO₄³⁻,^{a} 2 × Zn²⁺ | | 7 × PO₄³⁻,^{a} 2 × Zn²⁺ | |
| # water molecules | 2 | | | |
| Wilson B (/Å²) | 50.2 | | 44.3 | |
| Rmsd bond distances (/Å) | 0.008 | | 0.007 | |
| Rmsd bond angles (/°) | 1.65 | | 1.57 | |
| Ramachandran (COOT) | 97.5/1.4/1.1 | | 97.7/1.7/0.6 | |
| OK/OK-ish/Bad (/%) | | | | |

^{a}Likely a partially occupied and somewhat disordered I6P (inositol hexaphosphate).

### 6.9.1. Synthesis of Modified R- and S-Hydroxyazepan-2-One Nucleosides and their Phosphoramidites

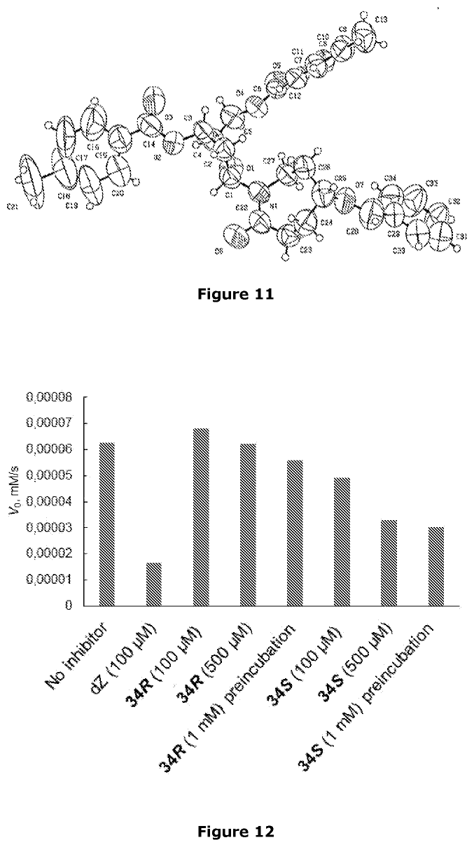

A method reported recently described the synthesis of compound 30 from the ketal 29 followed by Beckmann rearrangement to give the cyclic amide (31) (Scheme 8).⁵² The cyclic amide (31) and Hoffer's chlorosugar 1 were coupled in the presence of K-^{t}BuO in 1,4-dioxane at room temperature to provide a pair of isomers 32R and 32S with moderate yields (52 and 48%, respectively) and β:α ratio of 99:1. These isomers were purified using silica gel column chromatography. After purification, 32R was crystallized from 30% EtOAc in hexane. The stereochemistry of 32R was assigned based on X-ray crystal data (FIG. 11).

Here onwards, these isomers were treated by the same sequence of reactions separately. Benzyl deprotection was done by catalytic hydrogenation using 10% Pd/C, H₂ at room temperature to give 33R and 33S with 92 and 88%, respectively. One part of the benzyl-deprotected compounds was treated with 30% aq. NH₃, MeOH, r.t., 3 days to provide free nucleosides 34R and 34S (72 and 79%, respectively). The rest of the portion was treated with tetrabutyl diphenylsilyl (TBDPS) chloride in the presence of imidazole as a base in DCM to give 35R and 35S with yields 82 and 79%, respectively. Toluoyl groups on 35R and 35S were cleaved by 30% aq. NH₃, MeOH at r.t. for 3 days to provide 36R and 36S (90 and 85%, respectively). After the toluoyl deprotection, 5′-hydroxyl group was selectively protected by DMT chloride under standard DMT protection conditions to give 37R and 37S with 85 and 83% yields, respectively. Phosphoramidites 38R and 38S were prepared employing standard phosphitylation conditions with an isolated yield of 90 and 85%, respectively.

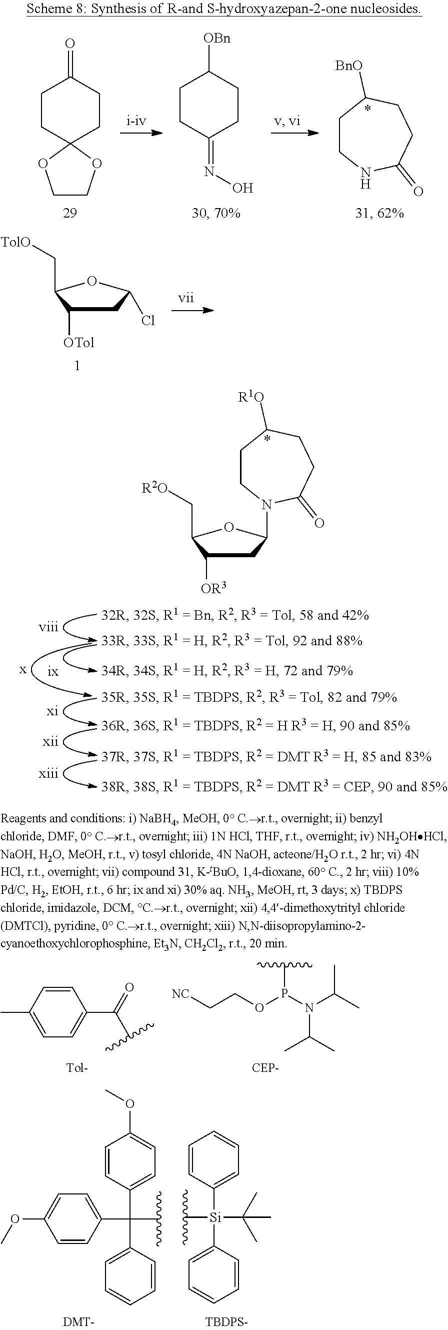

### 6.9.1.1. Synthesis of 5-benzyloxyazepan-2-one (31)

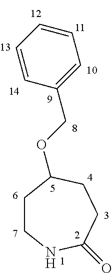 [SMILES: O=C1CCC(OCc2ccccc2)CCN1]

Synthesis of 31 was done from 29 which was prepared by the reported procedure (yield 7.6 g, 70%).⁵²

¹H NMR (500 MHz, CDCl₃) δ 7.36-7.32 (m, 4H, H-10, H-11, H-13, H-14), 7.30-7.25 (m, 1H, H-12), 6.69 (s, 1H, NH), 4.55, 4.51 (2d, 2H, J=11.90 Hz, H-8), 3.70-3.68 (m, 1H, H-5), 3.56-3.50 (m, 1H, H-7), 3.01-2.95 (m, 1H, H₇), 2.84-2.79 (m, 1H, H-4), 2.24-2.19 (m, 1H, H-4), 1.97-1.90 (m, 2H, H-6), 1.87-1.80 (m, 2H, H-3).

HRMS (ESI) m/z: [M+H]⁺ Calcd for C₁₃H₁₈NO₂ 220.1332. found 220.1331.

### 6.9.1.2. Synthesis of 1-[2-deoxy-3,5-bis-O-(4-methylbenzoyl)-β-D-erythro-pentofuranosyl]-(R)-5-benzyloxyazepan-2-one (34R) and 1-[2-deoxy-3,5-bis-O-(4-methylbenzoyl)-β-D-erythro-pentofuranosyl]-(S)-5-benzyloxyazepan-2-one (34S)

To a solution of the compound 31 (7.5 g, 0.99 mmol) in 250 mL of 1,4-dioxane was added K-^{t}BuO (5.8 g, 52 mmol) followed by Hoffer's chlorosugar (14.0 g, 36.0 mmol) and the solution was stirred at rt for 2 hr. After the consumption of the starting materials as observed by TLC, the reaction mixture was diluted with EtOAc (100 mL) and washed with water. Organic layer was separated, dried over anhydrous sodium sulfate, filtered, and concentrated in vacuo. The crude mixture was purified by column chromatography over silica gel eluting with 0-30% EtOAc in hexane to afford the desired pure isomers as a foam (yield 8.5 g, 64% of R isomer and 36% S isomer, with purity over 99% as 3-nucleosides as determined by ¹H NMR).

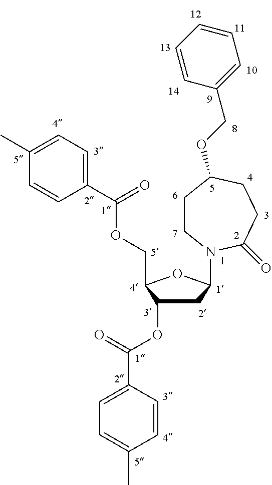

¹H NMR (500 MHz, CDCl₃) δ 7.94-7.92 (m, 4H, H-11, H-12, H-14, H-15), 7.35-7.32 (m, 2H, H-3″), 7.29-7.27 (m, 3H, H-3″, H-13), 7.26-7.23 (m, 4H, H-4″), 6.58 (dd, 1H, J=9.60, 5.40 Hz, H-1′), 5.52-5.50 (m, 1H, H-3′), 4.66 (dd, 1H, J=11.89, 3.45 Hz, H-5′), 4.53 (dd, 1H, J=11.89, 3.87 Hz, H-5′), 4.47, 4.43 (2d, 2H, J=11.90 Hz, H-8), 4.34 (td, 1H, J=3.45, 1.36 Hz, H-4′), 3.63 (bs, 1H, H-5), 3.61-3.58 (m, 1H, H-7), 3.14 (dd, 1H, J=15.69, 7.40 Hz, H-7), 2.94 (t, 1H, 12.65 Hz, H-3), 2.42 (s, 3H, CH₃), 2.41 (s, 3H, CH₃), 2.33-2.28 (m, 1H, H-2′a), 2.27-2.23 (m, 1H, H-3), 2.18-2.12 (m, 1H, H-2′b), 1.97-1.94 (m, 1H, H-6), 1.85-1.75 (m, 2H, H-6, H-4), 1.60-1.55 (m, 1H, H-4).

HRMS (ESI) m/z: [M+Na]⁺ Calcd for C₃₄H₃₇NO₇Na 594.2462. found 594.2466.

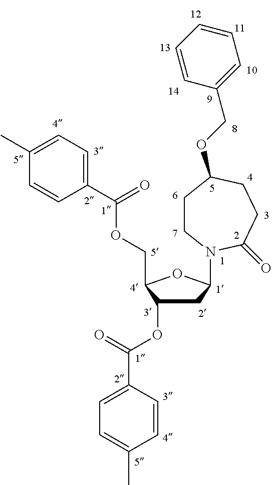

¹H NMR (500 MHz, CDCl₃) δ 7.94-7.93 (m, 4H, H-11, H-12, H-14, H-15), 7.35-7.33 (m, 2H, H-3″), 7.29-7.24 (m, 7H, H-3″, H-4″, H-13), 6.59 (dd, 1H, J=9.47, 5.46 Hz, H₁″), 5.54-5.52 (m, 1H, H-3′), 4.73 (dd, 1H, J=11.88, 3.26 Hz, H-5′), 4.51 (dd, 1H, J=11.88, 3.74 Hz, H-5′), 4.45, 4.41 (2d, 2H, J=11.80 Hz, H-8), 4.32 (td, 1H, J=3.11, 1.92 Hz, H-3′), 3.63-3.60 (m, 1H, H-5), 3.56-3.52 (m, 1H, H-7), 3.08-3.05 (m, 1H, H-7), 2.88-2.85 (m, 1H, H-3), 2.41 (s, 3H, CH₃), 2.41 (s, 3H, CH₃), 2.34-2.31 (m, 1H, H-3), 2.28-2.25 (m, 1H, H-2′a), 2.18-2.14 (m, 1H, H-2′b), 1.93-1.87 (m, 2H, H-4), 1.73-1.67 (m, 2H, H-6).HRMS (ESI) m/z: [M+Na]⁺ Calcd for C₃₄H₃₇NO₇Na 594.2462. found 594.2464.

### 6.9.1.3. Synthesis of 1-[2-deoxy-3,5-bis-O-(4-methylbenzoyl)-β-D-erythro-pentofuranosyl]-(R)-5-hydroxyazepan-2-one (33R) and 1-[2-deoxy-3,5-bis-O-(4-methylbenzoyl)-β-D-erythro-pentofuranosyl]-(S)-5-hydroxyazepan-2-one (33S)

After two vacuum/H₂ cycles to replace air inside the flask with hydrogen, the mixture of the substrate (5 g 8.8 mmol), 10% Pd/C (10 wt % of the substrate) in EtOH (100 mL) was stirred at room temperature under hydrogen (balloon) for 6 h. The reaction mixture was filtered using celite pad, and washed twice with 50 mL EtOH, the filtrate was concentrated in vacuo to provide the crude product. It was purified by column chromatography over silica gel eluting with 0-10% MeOH in DCM to afford the pure products as foams (yield 3.9 g, 93%).

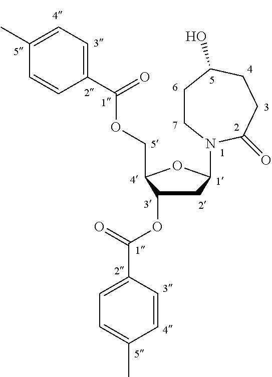

¹H NMR (500 MHz, CDCl₃) δ 7.93-7.90 (m, 4H, H-3″), 7.25-7.22 (m, 4H, H-4″), 6.57 (dd, 1H, J=9.56, 5.40 Hz, H-1′), 5.52-5.50 (m, 1H, H-3′), 4.65 (dd, 1H, J=11.89, 3.36 Hz, H-5′a), 4.52 (dd, 1H, J=11.88, 3.82 Hz, H-5′b), 4.33 (td, J=3.0, 1.60 Hz, H-4′), 3.93 (bs, 1H, H-5), 3.55 (dd, 1H, J=15.49, 9.14 Hz, H-7), 3.12 (dd, 1H, J=15.49, 8.05 Hz, H-7), 2.87-2.82 (m, 1H, H-3), 2.41 (s, 3H, CH₃), 2.40 (s, 3H, CH₃), 2.34-2.29 (m, 1H, H-4), 2.25 (dd, 1H, J=13.80, 5.24 Hz, H-2′a), 2.17-2.11 (m, 1H, H-2′b), 1.92 (t, 1H, J=12.24 Hz, H-3), 1.79-1.76 (m, 1H, H-4), 1.74-1.68 (m, 1H, H-6), 1.64-1.58 (m, 1H, H-6).

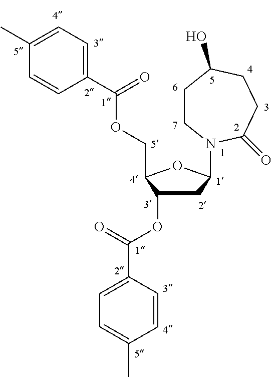

¹H NMR (500 MHz, CDCl₃) δ 7.93-7.90 (m, 4H, H-3″), 7.25-7.23 (m, 4H, H-4″), 6.57 (dd, 1H, J=9.52, 5.52 Hz, H-1′), 5.52-5.50 (m, 1H, H-3′), 4.67 (dd, 1H, J=11.90, 3.25 Hz, H-5′a), 4.51 (dd, 1H, J=11.90, 3.80 Hz, H-5′b), 4.31 (td, 1H, J=3.20, 1.70 Hz, H-4′), 3.96-3.92 (m, 1H, H-5), 3.54 (dd, 1H, J=15.47, 9.02 Hz, H-7), 3.08 (dd, 1H, J=15.47, 8.32 Hz, H-7), 2.81 (t, 1H, J=12.35 Hz, H-3), 2.41 (s, 3H, CH₃), 2.40 (s, 3H, CH₃), 2.37-2.33 (m, 1H, H-4), 2.26 (dd, 1H, 13.62, 5.17 Hz, H-2′a), 2.19-2.13 (m, 1H, H-2′b), 1.94 (t, 1H, J=12.10 Hz, H-3), 1.84-1.78 (m, 2H, H-6), 1.75-1.69 (m, 1H, H-4), 1.53-1.47 (m, 1H, H-6).

### 6.9.1.4. Synthesis of 1-{2-deoxy-3,5-di-O-(4-methylbenzoyl)-3-D-erythro-pentofuranosyl}-[(tert-butyldiphenylsilyl)-(R)-5-oxy]azepan-2-one (34R) and 1-{2-deoxy-3,5-di-O-(4-methylbenzoyl)-β-D-erythro-pentofuranosyl}-[(tert-butyldiphenylsilyl)-(S)-5-oxy]azepan-2-one (34S)

To a stirring solution of 33R or 33S (1.8 g, 4.0 mmol) in dry DCM (100 mL) at 0° C. was added imidazole (1.0 g, 14.7 mmol) followed by TBDPS chloride (1.46 mL, 3.6 mmol) dropwise. After the addition, the reaction mixture was slowly brought to room temperature and continued stirring for 6 hr. The reaction was monitored by TLC, after consumption of the starting material, 100 mL DCM was added to the reaction mixture and washed with water followed by brine. The organic layer was separated, dried over anhydrous sodium sulfate, filtered, and concentrated in vacuo. The crude product was purified by column chromatography over silica gel eluting with 0-30% EtOAc in hexane to afford the product as a foam (yield 2.3 g, 85%).

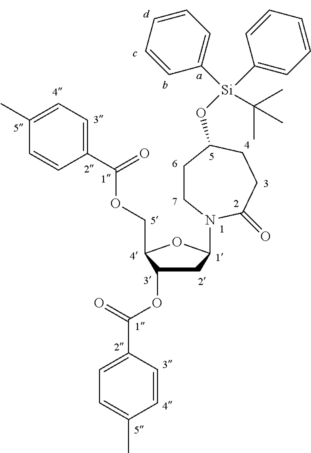

¹H NMR (500 MHz, CDCl₃) δ 7.93-7.89 (in, 4H, H-c), 7.61-7.58 (in, 4H, H-b) 7.44-7.40 (m, 2H, H-d), 7.37-7.32 (m, 4H, H-3″), 7.25-7.18 (m, 4H, H-4″), 6.58 (dd, 1H, J=9.62, 5.32 Hz, H-1′), 5.50 (dt, 1H, J=3.40, 2.55 Hz H-3′); 4.67 (dd, 1H, J=11.90, 3.40 Hz, H-5′a), 4.56 (dd, 1H, J=9.52, 5.52 Hz, H-5′b), 4.35 (td, 1H, J=3.30, 1.80 Hz, H-4′), 4.00 (s, 1H, H-5), 3.76 (td, 1H, J=14.62, 5.3 Hz, H-7) 3.17-3.08 (m, 2H, H-7, H-3), 2.41 (2s, 6H, CH₃), 2.24 (dd, 1H, J=14.18, 8.46 Hz, H-3), 2.20 (dd, 1H, J=14.55, 6.7 Hz, H-2′a); 2.10 (ddd, 1H, J=16.30, 9.60, 6.7 Hz, H-2′b), 1.78-1.73 (m, 1H, H-4), 1.65-1.59 (m, 2H, H-6, H-4), 1.39 (t, 1H, J=10.97 Hz, H-6), 1.04 (s, 9H, CH₃).

HRMS (ESI) m/z: [M+H]⁺ Calcd for C₄₃H₅₀NO₇Si 720.3351. found 720.3340.

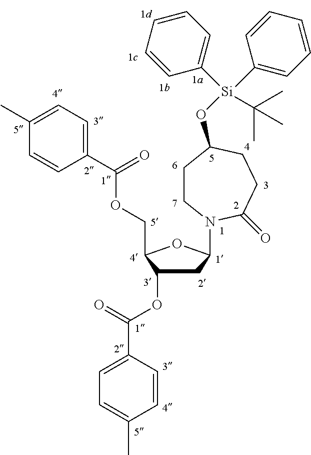

¹H NMR (500 MHz, CDCl₃) δ 7.94-7.91 (m, 4H, H-c) 7.59-7.54 (m, 4H, H-b), 7.44-7.40 (m, 2H, H-d), 7.35-7.32 (m, 4H, H-3″) 7.25-7.20 (m, 4H, H-4″), 6.57 (dd, 1H, J=9.27, 5.58 Hz, H-1′), 5.54-5.52 (m, 1H, H-3′), 4.67 (dd, 1H, J=11.91, 3.16 Hz, H-5′a), 4.48 (dd, 1H, J=11.91, 3.47 Hz, H-5′b), 4.29 (dt, 1H, J=3.40, 2.0 Hz, H-4′), 3.94 (s, 1H, H-5)), 3.67-3.62 (m, 1H, H-7), 3.08-3.03 (m, 1H, H-7), 2.96 (bs, 1H, H-3), 2.41 (s, 3H, CH₃), 2.37 (s, 3H, CH₃), 2.28 (dd, 1H, J=13.85, 5.60 Hz, H-2′a), 2.27 (d, 1H, H-3); 2.25-2.17 (m, 2H, H-2′b), 1.72-1.68 (m, 2H, H-4) 1.61-1.52 (m, 2H, H-6), 1.03 (s, 9H, CH₃).HRMS (ESI) m/z: [M+H]⁺ Calcd for C₄₃H₅₀NO₇Si 720.3351. found 720.3350.

### 6.9.1.5. Synthesis of 1-[2-deoxy-β-D-erythro-pentofuranosyl]-(R)-5-hydroxyazepan-2-one (34R) and 1-[2-deoxy-β-D-erythro-pentofuranosyl]-(S)-5-hydroxyazepan-2-one (34S)

Toluoyl protected 2′-deoxynucleoside 33R or 33S (6.10 g, 13.6 mmol) was dissolved in MeOH (500 ML) and aq. ammonia (28%, 50 mL) was added in one portion. Reaction mixture was stirred at room temperature for 48 h, evaporated all the volatiles in vacuo, co-evaporated with H₂O (2×200 mL), MeOH (200 mL) several times to get the desired products (yield 3 g, 96%).

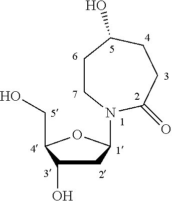

¹H NMR (500 MHz, D₂O) δ 6.33 (dd, 1H, J=8.0, 5.9 Hz, H-1′), 4.30-4.34 (m, 1H, H-3′) 4.01-3.95 (m, 1H, H-5), 3.88 (td, 1H, J=4.0, 2.10 Hz, H-4′), 3.76 (dd, 1H, J=12.24, 3.67 Hz, H-5′a), 3.69 (dd, 1H, J=12.24, 5.43 Hz, H-5′b), 3.58 (dd, 1H, J=15.75, 8.53 Hz, H-7), 3.30 (dd, 1H, J=15.75, 9.04 Hz, H-7), 2.71 (t, 1H, J=10.77 Hz, H-3), 2.48-2.41 (m, 1H, H-3), 2.18-2.12 (m, 1H, H-4), 2.07-2.04 (m, 1H, H-2′a), 2.03-1.95 (m, 3H, H-2′b, H₄), 1.69-1.57 (m, 2H, H-6).

HRMS (ESI) m/z: [M+Na]⁺ Calcd for C₁₁H₁₉NO₅Na 268.1155. found 268.1150.

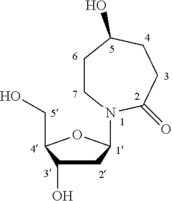

¹H NMR (500 MHz, D₂O) δ 6.33 (dd, 1H, J=8.47, 6.32 Hz, H-1′), 4.37-4.34 (m, 1H, H-3′) 3.98-3.94 (m, 1H, H-5), 3.89 (td, 1H, J=4.3, 2.20 Hz, H-4′), 3.75 (dd, 1H, J=12.26, 4.03 Hz, H-5′a), 3.68 (dd, 1H, J=12.26, 5.57 Hz, H-5′b), 3.59 (dd, 1H, J=15.89, 7.97 Hz, H-7), 3.27 (dd, 1H, J=15.89, 9.77 Hz, H-7), 2.67-2.51 (m, 2H, H-3), 2.21-2.15 (m, 1H, H-4), 2.08-1.98 (m, 2H, H-2′), 1.63-1.48 (m, 3H, H₄, H-6).

HRMS (ESI) m/z: [M+Na]⁺ Calcd for C₁₁H₁₉NO₅Na 268.1155. found 268.1151.

### 6.9.1.6. Synthesis of 1-[2-deoxy-β-D-erythro-pentofuranosyl]-(R)-5-[(tert-butyldiphenylsilyl)oxy]azepan-2-one (36R) and 1-[-2-deoxy-β-D-erythro-pentofuranosyl]-(S)-5-[(tert-butyldiphenylsilyl)oxy]azepan-2-one (36S)

Compound 37R or 37S (1.2 g, 2.5 mmol) was dissolved in MeOH (100 mL) and aq. ammonia (28%, 15 mL) was added in one portion. Reaction mixture was stirred at room temperature for 48 h, evaporated all the volatiles, co-evaporated with H₂O (2×100 mL), MeOH (100 mL) several times to get 38R or 38S as pure compound (yield 0.72 g, 89%).

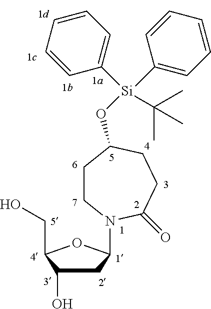

¹H NMR (500 MHz, CDCl₃) δ 7.63-7.62 (m, 4H, H-1b), 7.44-7.41 (m, 2H, H-1d); 7.38-7.36 (m, 4H, H-1c); 6.27 (dd, 1H, J=8.20, 6.50 Hz, H-1′); 4.32-4.29 (m, 1H, H-3′); 4.05 (s, 1H, H-5); 3.82 (td, 1H, J=3.80, 2.80 Hz, H-4′); 3.81-3.79 (m, 1H, H-7); 3.76 (dd, 1H, J=11.60, 3.75 Hz, H-5′a); 3.70 (dd, 1H, J=11.60, 4.05, H-5′b); 3.50 (s, 1H, 3′-OH); 3.10 (d, J=6.95 Hz, 1H, H-7); 3.17 (d, J=6.80 Hz, 1H, H-3); 2.93 (s, 1H, 5′-OH); 2.20 (dd, 1H, J=14.12, 8.47 Hz, H-3); 2.05-1.99 (m, 1H, H-2′a); 1.96-1.92 (ddd, J=13.30, 6.05, 2.90 Hz, 1H, H-2′b); 1.78-1.72 (m, 2H, H-6, H-4); 1.63 (t, 1H, J=13.20 Hz, H-4); 1.49 (m, 1H, H-6); 1.07 (s, 9H, CH₃).

HRMS (ESI) m/z: [M+H]⁺ Calcd for C₂₇H₃₈NO₅Si 484.2514. found 484.2514.

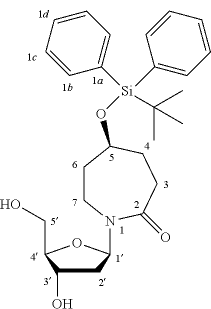

¹H NMR (500 MHz, CDCl₃) δ 7.63-7.61 (m, 4H, H-1b); 7.44-7.41 (m, 2H, H-1d); 7.38-7.35 (m, 4H, H-1c); 6.27 (dd, 1H, J=7.80, 6.40 Hz, H-1′); 4.31-4.28 (m, 1H, H-3′); 4.00 (s, 1H, H-5); 3.79 (td, 1H, J=4.05, 2.80 Hz, H-4′); 3.76-3.72 (m, 1H, H-7); 3.70 (dd, 1H, J=11.40, 3.30 Hz, H-5′a); 3.64 (dd, 1H, J=11.40, 4.05 Hz, H-5′b); 3.59 (s, 1H, 3′-OH); 3.03 (dd, 1H, J=15.55, 6.90 Hz, H-7); 3.01-2.95 (m, 1H, H-3); 2.93 (s, 1H, 5′-OH); 2.23-2.18 (m, 1H, H-3); 2.10-2.04 (m, 1H, H-2′a); 2.0 (ddd, 1H, J=13.55, 6.30, 3.20 Hz, H-2′b); 1.74-1.61 (m, 4H, H-6, H-4); 1.07 (s, 9H, CH₃).

HRMS (ESI) m/z: [M+H]⁺ Calcd for C₂₇H₃₈NO₅Si 484.2514. found 484.2517.

### 6.9.1.7. Synthesis of 1-{2-deoxy-5-O-(4,4′-dimethoxytrityl)-β-D-erythro-pentofuranosyl}-(R)-5-[(tert-butyldiphenylsilyl)oxy]azepan-2-one (37R) and 1-{2-deoxy-5-O-(4,4′-dimethoxytrityl)-β-D-erythro-pentofuranosyl}-(S)-5-[(tert-butyldiphenylsilyl)oxy]azepan-2-one (37S)

To a stirring solution of a nucleoside 36R or 36S (0.4 g, 1.16 mmol) in dry pyridine (10 mL), 4,4′-O-dimethoxytrityl chloride (0.39 g, 1.15 mmol) was added at 0° C. and the mixture was stirred at r.t. overnight under argon. After consumption of the starting nucleoside, water (1 mL) was added. Solvents were evaporated in vacuo and the residue was dissolved in 50 mL DCM and washed with brine (2×10 mL). Organic layer was dried over anhydrous sodium sulfate, filtered, and concentrated in vacuo. The crude product was purified by column chromatography over silica gel treated with 10% Et₃N in DCM and eluted with 0-5% MeOH in DCM to afford the desired products as foams (0.68 g, 90%).

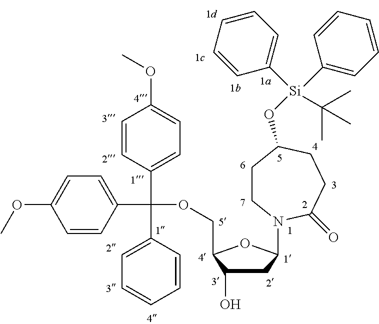

¹H NMR (500 MHz, CDCl₃) δ 7.62-7.61 (m, 2H, H-b); 7.57-7.55 (m, 2H, H-b); 7.44-7.40 (m, 3H, H-d, H-4″); 7.37-7.29 (m, 7H, H-c, H-2″, H-2′″); 7.27-7.19 (m, 5H, H-c, H-2′″); 6.84-6.81 (m, 4H, H-3′″); 6.51 (dd, 1H, J=8.10, 6.55 Hz, H-1′); 4.38-4.36 (m, 1H, H-3′); 4.01 (s, 1H, H-5); 3.89 (td, 1H, J=3.90, 2.20 Hz, H-4′); 3.78 (s, 6H, OCH₃), 3.32 (m, 2H, H-5′); 3.12 (bs, 1H, H-4); 2.81 (m, 2H, H-7); 2.23 (dd, 1H, J=13.99, 8.56 Hz, H-4); 2.03-1.96 (m, 2H, H-2′); 1.80-1.74 (m, 1H, H-3); 1.71-1.63 (m, 2H, H-6, H-3); 1.48-1.40 (m, 1H, H-6); 1.03 (s, 9H, CH₃).

HRMS (ESI) m/z: [M+Na]⁺ Calcd for C₄₈H₅₅NNaO₇Si 808.3640. found 808.3640.

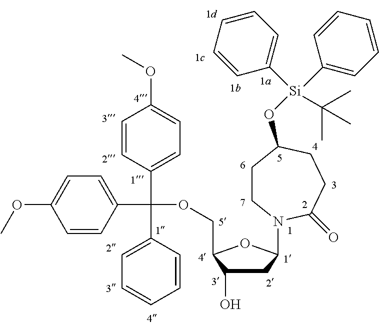

¹H NMR (500 MHz, CDCl₃) δ 7.62-7.61 (m, 2H, H-b); 7.58-7.57 (m, 2H, H-b); 7.44-7.39 (m, 3H, H-d, H-2″); 7.38-7.34 (m, 3H, H-2″, H-c); 7.31-7.28 (m, 6H, H-2′″, H-3″); 7.25-7.18 (m, 3H, H₄″, H-c); 6.82-6.79 (m, 4H, H-3′″); 6.48 (dd, 1H, J=8.10, 6.55 Hz, H-1′); 4.40 (m, 1H, H-3′); 4.00 (s, 1H, H-5); 3.85 (td, 1H, J=3.50, 2.15 Hz, H-4′); 3.78 (2s, 6H, OCH₃), 3.33-3.25 (m, 2H, H-5′); 3.20-3.17 (m, 1H, H-4); 2.99 (bs, 1H, H-4); 2.78 (m, 2H, H-7); 2.28-2.23 (m, 1H, H-6); 2.13-2.07 (m, 2H, H₂′); 1.78-1.73 (m, 1H, H-3); 1.66-1.62 (m, 2H, H-6, H-3); 1.08 (s, 9H, CH₃).

HRMS (ESI) m/z: [M+Na]⁺ Calcd for C₄₈H₅₅NNaO₇Si 808.3640. found 808.3633.

### 6.9.1.8. Synthesis of 1-{3-O—(N,N-diisopropylamino-2-cyanoethoxyphosphanyl)-2-deoxy-5-O-(4,4′-dimethoxytrityl)-β-D-erythro-pentofuranosyl}-(R)-5-[(tert-butyldiphenylsilyl)oxy]azepan-2-one (38R) and 1-{3-O—(N,N-diisopropylamino-2-cyanoethoxyphosphanyl)-2-deoxy-5-O-(4,4′-dimethoxytrityl)-β-D-erythro-pentofuranosyl}-(S)-5-[(tert-butyldiphenylsilyl)oxy]azepan-2-one (38S)

To a stirring solution of 37R or 37S (0.4 g, 0.66 mmol) in dry DCM (10 mL) under argon at rt were added Et₃N (0.12 ml, 0.86 mmol) followed by 2-cyanoethyl-N,N-diisopropyl chlorophosphoramidite (0.17 g, 0.71 mmol). After the disappearance of the starting material on TLC in 10 min the reaction mixture was washed with saturated sodium bicarbonate solution (2×5 mL) followed by brine (5 mL). The organic layer was dried over anhydrous sodium sulfate, filtered, and the combined fractions were evaporated in vacuo. The crude product was purified by column chromatography over silica gel (60-120 mesh) saturated with Et₃N (10%) and eluted with CH₂Cl₂/acetone (9:1) to give a white foam (0.45 g, 85%).

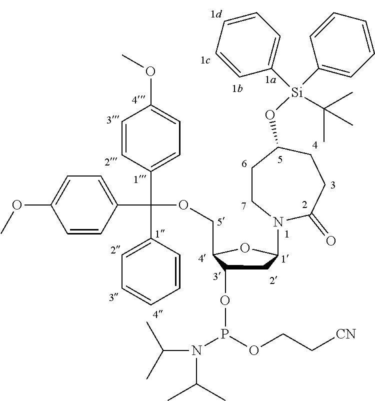

¹H NMR (500 MHz, DMSO-d₆) δ 7.59-7.55 (m, 2H, H-b); 7.51-7.48 (m, 2H, H-b); 7.45-7.41 (m, 1H, H-4″); 7.40-7.35 (m, 6H, H-2″ H-c); 7.29-7.19 (m, 8H, H-2′″, H-3″, H-d); 6.88-6.83 (m, 4H, H-3′″); 6.25 (dd, 1H, J=8.45, 6.10 Hz, H-1′); 4.44-4.38 (m, 1H, H-3′); 3.98-4.02 (m, 1H, H-5); 3.92-3.85 (m, 1H, H-4′); 3.70 (s, 6H, OCH₃); 3.62-3.57 (m, 2H, CH₂CH₂CN); 3.57-3.55 (m, 1H, H-7); 3.54-3.43 (m, 2H, NCHCH₃); 3.29 (bs, 1H, H-4); 3.19-3.09 (m, 2H, H-5′); 2.73, 2.62 (2t, J=5.90 Hz, CH₂CH₂CN); 2.21-2.15 (m, 1H, H-7); 2.08-2.10 (m, 1H, H-2′a); 1.97-1.86 (m, 1H, H-2′b); 1.69-1.59 (m, 2H, H-3); 1.58-1.52 (m, 2H, H-6); 1.19-1.06 (m, 12H, NCHCH₃); 0.96 (s, 9H, CH₃).

³¹P NMR (202.5 MHz, DMSO-d₆, ref. 85% H₃PO₄) δ 147.10, 146.63 in −1:1 ratio.

HRMS (ESI) m/z: [M+H]⁺ Calcd for C₅₇H₇₃N₃O₈PSi 986.4899. found 986.5901.

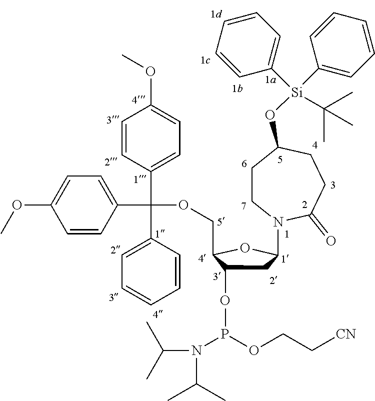

¹H NMR (500 MHz, DMSO-d₆) δ 7.57-7.55 (in, 2H, H-b); 7.53-7.51 (in, 2H, H-b); 7.45-7.41 (in, 1H, H-4″); 7.40-7.30 (in, 7H, H-2″, H-c, H-d); 7.25-7.17 (in, 6H, H-2′″, H-3″); 6.85-6.81 (in, 4H, H-3′″); 6.23 (dd, 1H, J=8.00, 6.00 Hz, H-1′); 4.44-4.38 (in, 1H, H-3′); 3.95 (bs, 1H, H-5); 3.87-3.81 (in, 1H, H-4′); 3.70 (s, 6H, OCH₃); 3.61-3.57 (in, 2H, CH₂CH₂CN); 3.56-3.44 (in, 2H, NCHCH₃); 3.18-3.11 (in, 2H, H-5′); 3.10-3.05 (1H, H-4); 2.75, 2.70 (bs, 1H, H-7); 2.62 (2t, J=5.84 Hz, CH₂CH₂CN); 2.28-2.23 (1H, H-7); 2.14-2.08 (in, 1H, H-2′a); 2.03-1.92 (in, 1H, H-2′b); 1.73-1.68 (in, 1H, H-3); 1.64-1.57 (in, 2H, H-6, H-3); 1.54-1.47 (in, 1H, H-6); 1.12-1.08 (in, 9H, NCHCH₃); 1.00 (s, 9H, CH₃); 0.97, 0.96 (2s, NCHCH₃).

³¹P NMR (202.5 MHz, DMSO-d₆, ref. 850/H₃PO₄) δ 147.17, 146.72 in ˜1:1 ratio.

### 6.9.2. Synthesis of Diazapin-2-One Nucleoside and its Phosphoramidite

Diazapin-2-one nucleoside was reported previously as a tight-binding inhibitor of CDA. The synthesis started with the formation of diallyl urea (40, Scheme 9) by the condensation of allylamine (39) in presence of N,N-disuccinimidyl carbonate in THF. Coupling of 40 to Hoffer's chlorosugar 1 was performed by previously described silyl modification of the Hilbert-Johnson reactions⁵³ using SnCl₄ as Lewis acid in dichloroethane at −35° C. to furnish 41 with moderate yield (45%) and 13:a ratio of 9:1. The 3 isomer as a major product was confirmed by NOESY NMR experiment. The NOESY spectrum shows cross-peak of 1′-proton (6.43 ppm) and 4′-proton (4.30 ppm) of the major isomer of compound 41 through space, confirming it as a 1-nucleoside, while the minor α-anomer had a cross-peak between 1′ and 3′-protons (6.36 and 5.00 ppm, respectively).

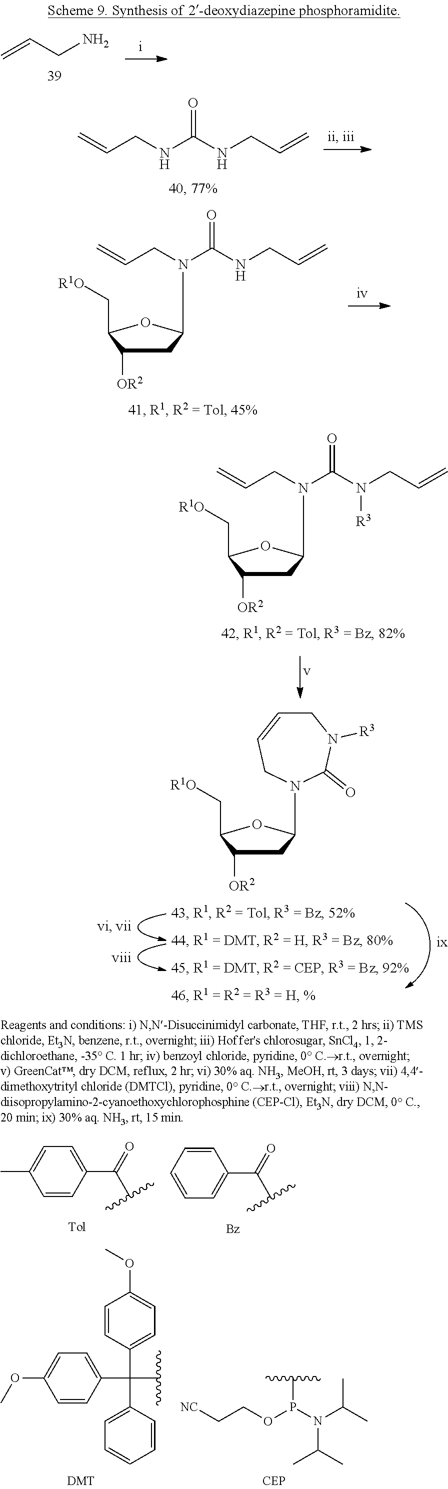

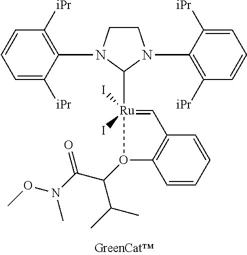

For alkylation of the amide as reported in the literature it was necessary to lock the allyl groups oriented in trans orientation for the successful ring-closure metathesis (RCM).⁵⁴ Inventors protected the amide (41) with benzoyl chloride to provide 42 and performed RCM with GreenCat™, which provided the required seven-membered ring (43) with 52% isolated yield. Resuspending and washing the precipitate in methanol provided >99% pure P anomer. Toluoyl groups on 43 were selectively deprotected by 30% aq. NH₃ in MeOH at r.t. for 3 days followed by selective protection of 5′-hydroxyl group using standard DMT protection conditions to provide 44 (80% yield). Phosphoramidite 45 was prepared employing standard phosphitylation conditions with an isolated yield of 92%. A deprotected nucleoside 46 was obtained from compound 43 using 30% aq. ammonia solution at rt, for 15 min.

### 6.9.2.1. Synthesis of Diallyl Urea (40)

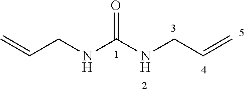

To a stirring solution of allylamine (1 g, 17 mmol) in dry THF (15 mL), N,N-disuccinimidyl carbonate (2.25 g, 9 mmol) was added at r.t. and the mixture was stirred at r.t. for 2 hr under argon. After consumption of the starting amine (TLC analysis), solvent was evaporated in vacuo. The crude product was purified by column chromatography over silica gel eluting with 0-50% EtOAc in hexane to afford the desired product as a white solid (yield 1.9 g, 77%).

¹H NMR (500 MHz, CDCl₃) δ 5.84-5.76 (m, 2H, H-4); 5.85 (bs, 2H, NH); 5.14 (dd, 2H, J=17.20, 1.55 Hz, H-5a); 5.04 (dd, 2H, J=10.25, 1.55 Hz, H-5b); 3.75-3.72 (m, 4H, H-3).

HRMS (ESI) m/z: [M+H]⁺ Calcd for C₇H₁₃N₂₀ 141.1022. found 141.1021.

### 6.9.2.2. Synthesis of 1-[2-deoxy-3,5-bis-O-(4-methylbenzoyl)-β-D-erythro-pentofuranosyl]diallylurea (41)

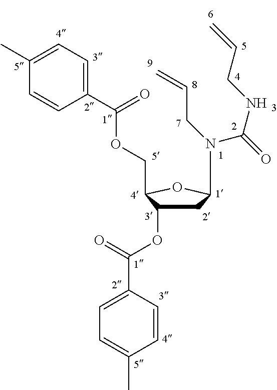

To the solution of the compound 40 (2.0 g, 14.0 mmol) in benzene (30 mL) NEt₃ (3.38 mL, 24 mmol) and trimethylsilyl chloride (2.2 mL, 17 mmol) were sequentially added at r.t. under argon. The reaction mixture was stirred overnight, and then filtered over sintered funnel. Solvents were evaporated in vacuo to afford silyl protected diallyl urea derivative. To the solution of silyl-protected diallyl urea (2.5 g, 9.0 mmol) in dichloroethane (60 mL) freshly distilled SnCl₄ (3.36 mL, 28 mmol) and Hoffer's chlorosugar 1 (3.36 g, 8.6 mmol) were sequentially added at −35° C. Reaction mixture was stirred for 1.5 h at −35° C. and after the consumption of starting silylated diallyl amine, pyridine (10 mL) and H₂O (50 mL) were added and reaction mixture was stirred at r.t. for 1h followed by addition of H₂O (100 mL). The resulting mixture was extracted with DCM (4×100 mL), combined organic layers were dried over Na₂SO₄, filtered and the combined organic fractions were evaporated in vacuo. The crude product was purified by flash chromatography on silica eluting with 0-30% EtOAc in hexane to afford the product as a form (yield 2 g, 45%, β/α=9:1).

¹H NMR (500 MHz, CDCl₃) δ 7.93-7.89 (m, 4H, H-3″); 7.26-7.21 (m, 4H, H-4″); 6.42 (dd, 1H, J=8.70, 6.85 Hz, H-1′); 5.87-5.76 (m, 2H, H-5, H-8); 5.50 (dt, 1H, J=8.70, 6.85 Hz, H-3′); 5.26-5.22 (m, 2H, H-9); 5.14-5.12 (m, 1H, H-6a); 5.09-5.08 m, 1H, H-6b); 4.90 (t, 1H, J=5.28 Hz, H-3); 4.61 (dd, 1H, J=11.90, 3.50 Hz, H-5′a); 4.55 (dd, 1H, J=11.90, 3.64 Hz, H-5′b); 4.36 (m, 1H, H-4′); 3.99-3.95 (m, 1H, H-4a); 3.86-3.84 (m, 2H, H-7), 3.80-3.77 (m, 1H, H-4b); 2.43 (2s, 6H, CH₃); 2.26-2.24 (m, 2H, H-2′).

### 6.9.2.3. Synthesis of 3-[2-deoxy-3,5-bis-O-(4-methylbenzoyl)-3-D-erythro-pentofuranosyl]-1-benzoyl-1,3,4,7-tetrahydro-2H-1,3-diazepin-2-one (43)

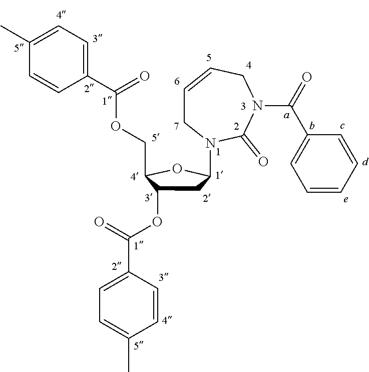

To a stirring solution of the compound 41 (4 g, 8.0 mmol) in pyridine (100 mL) at 0° C. NEt₃ (4.8 mL, 24 mmol) and benzoyl chloride (2.83 mL, 24 mmol) were added sequentially, and the reaction mixture was allowed to stir overnight under r.t. After the consumption of starting material by observing TLC, pyridine was evaporated in vacuo. The residue was resuspended in 100 mL ethyl acetate, washed with brine (2×10 mL), the organic layer was dried over anhydrous sodium sulfate, filtered, and concentrated in vacuo. The crude product was purified by column chromatography over silica gel eluting with 0-30% EtOAc in hexane to afford the desired product as a foam (yield 4 μm, 82%).

To a solution of the benzoyl-protected compound 42 (5 g, 8.0 mmol), in dry dichloromethane was added GreenCat™ (10 mol %) and reaction mixture was refluxed for 2 hr. After the consumption of the starting material, solvent was evaporated in vacuo and the crude product was purified by silica gel column chromatography eluting with 0-40% EtOAc in hexane to yield the desired product as a form (yield 1.9 g, yield 52%). The material was resuspended and washed with methanol to afford pure P product in >99% purity.

¹H NMR (500 MHz, CDCl₃) δ 7.98-7.95 (m, 2H, H-3″); 7.89-7.86 (m, 2H, H-3″); 7.56-7.53 (m, 2H, H-c); 7.47-7.44 (m, 1H, H-e); 7.40-7.35 (m, 2H, H-d); 7.31-7.27 (m, 2H, H-4″); 7.23-7.20 (m, 2H, H-4″); 6.19 (dd, 1H, J=9.25, 5.25 Hz, H-1′); 5.82-5.76 (m, 1H, H-5); 5.70-5.65 (m, 1H, H-6); 5.55 (m, 1H, H₃′); 4.74 (dd, 1H, J=15.35, 12.05 Hz, H-5′a); 4.68-4.62 (m, 1H, H₇); 4.57 (dd, 1H, J=15.60, 12.05 Hz, H-5′b); 4.35 (td, 1H, J=3.27, 2.45 Hz, H-4′); 4.30-4.23 (m, 1H, H-7); 4.08 (dd, 1H, J=19.15, 16.85 Hz, H-4); 3.96 (dd, 1H, J=21.90, 16.85 Hz, H-4); 2.44, 2.40 (2s, 6H, CH₃); 2.31 (ddd, 1H, J=16.00, 6.70, 5.25 Hz, H-2′b); 22.18 (ddd, 1H, J=15.35, 9.25, 6.70 Hz, H-2′a).

HRMS (ESI) m/z: [M+Na]⁺ Calcd for C₃₃H₃₂N₂NaO₇ 591.2102. found 591.2097.

### 6.9.2.4. Synthesis of 1-[2-deoxy-5-O-(4,4′-dimethoxytrityl)-3-D-erythro-pentofuranosyl]-3-benzoyl-1,3,4,7-tetrahydro-2H-1,3-diazepin-2-one (44)

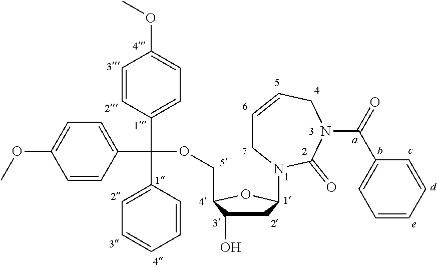

To a stirring solution of the compound 43 (4 g, 7.0 mmol) in 400 mL methanol was added 40 mL of 30% aq. ammonia solution and kept stirring for 3 days. After the disappearance of the starting material, volatiles were removed by rotary vacuum evaporator and residue was co-evaporated again with 100 mL water to remove formed methyl toluate and then freeze dried from water 50 mL to get the deprotected compound (2.25 g). This product was used without further purification to protect it with DMT. To a stirring solution of the deprotected product (2 g, 6.0 mmol) in dry pyridine (40 mL) at 0° C. 4,4′-dimethoxytrityl chloride (3.72 g, 11 mmol) was added and mixture was stirred at r.t. overnight. Pyridine was evaporated in vacuo. The residue was dissolved in 50 mL ethyl acetate and washed with brine (2×10 mL). The organic layer was dried over anhydrous sodium sulfate, filtered, and evaporated in vacuo. The crude product was purified by column chromatography over silica gel treated with 10% Et₃N in DCM and compound was eluted with 0-20% acetone in DCM to afford the desired product 46 as a foam (3.13 g, 80%).

¹H NMR (500 MHz, CDCl₃) δ 7.57-7.54 (m, 2H, H-c); 7.46-7.42 (m, 3H, H-2″, H-e); 7.40-7.36 (m, 2H, H-d); 7.34-7.30 (m, 6H, H-3′″, H₃″); 7.27-7.24 (m, 1H, H-4″); 6.87-6.83 (m, 4H, H-3′″); 6.05 (app t, 1H, J=7.05 Hz, H-1′); 5.79-5.75 (m, 1H, H-5); 5.70-5.66 (m, 1H, H-6); 4.62-4.55 (m, 1H, H-7); 4.43 (td, 1H, J=4.75, 2.80 Hz, H-4′); 4.40-4.34 (m, 1H, H-7); 4.12 (dd, 1H, J=19.35, 16.80 Hz, H-4); 3.96 (dd, 1H, J=21.65, 16.80 Hz, H-4); 3.87 (td, 1H, J=3.75, 2.20 Hz, H-3′); 3.80 (s, 6H, OCH₃); 3.40-3.31 (m, 2H, H-5′); 2.05 (dd, 2H, J=6.85, 5.20 Hz, H-2′).

HRMS (ESI) m/z: [M+Na]⁺ Calcd for C₃₃H₃₂N₂NaO₇ 591.2102. found 591.2097.

### 6.9.2.5. Synthesis of 1-[3-O—(N,N-diisopropylamino-2-cyanoethoxyphosphanyl)-2-deoxy-5-O-(4,4′-dimethoxytrityl)-β-D-erythro-pentofuranosyl]-3-benzoyl-1,3,4,7-tetrahydro-2H-1,3-diazepin-2-one (45)

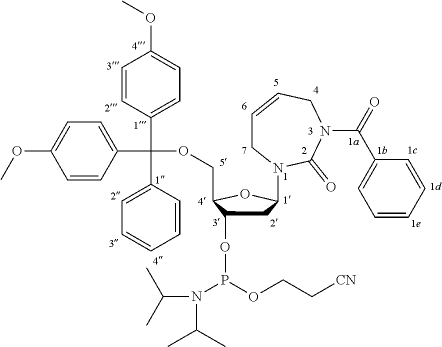

To a stirring solution of 5′-O-DMT protected compound 44 (0.20 g, 0.36 mmol) in dry DCM (10 mL) under argon at r.t., were added Et₃N (0.07 mL, 0.50 mmol) followed by 2-cyanoethyl N,N-diisopropyl chlorophosphoramidite (0.09 g, 0.38 mmol). After the disappearance of the starting material in 10 min the reaction mixture was washed with saturated sodium bicarbonate solution (2×5 mL) followed by brine (5 mL). The organic layer was dried over anhydrous sodium sulfate collum, filtered and the combined fractions were evaporated in vacuo. The crude product was purified by column chromatography over silica gel saturated with Et₃N (10%) in DCM and eluting with CH₂Cl₂/acetone (9:1) to give the desired product as a white foam (yield 0.32 μm, 92%).

¹H NMR (500 MHz, CDCl₃) δ 7.62-7.59 (m, 2H, H-c); 7.51-7.46 (m, 3H, H-2″, H-e); 7.45-7.41 (m, 2H, H-d); 7.38-7.31 (m, 6H, H-2′″, H-3″); 7.29-7.26 (m, 1H, H-4″); 6.87-6.83 (m, 4H, H-3′″); 6.10 (app t, 1H, J=7.50 Hz, H-1′); 5.83-5.79 (m, 1H, H-5); 5.74-5.68 (m, 1H, H-6); 4.68-4.63 (m, 1H, H-7); 4.62-4.57 (m, 1H, H-4′); 4.46-4.35 (m, 1H, H-7); 4.25-4.15 (m, 1H, H-4); 4.13-4.08 (m, 1H, H-4); 4.05, 4.02 (2td, 1H, J=3.50, 1.80 Hz, H-3′); 3.82, 3.83 (2s, 6H, OCH₃); 3.75-3.67 (m, 1H, NCHCH₃); 3.66-3.60 (m, 1H, NCHCH₃); 3.58-3.51 (m, 2H, 2H, CH₂CH₂CN); 3.47, 3.43 (2dd, 1H, J=10.35, 2.85 Hz, H-5′a); 3.31-3.26 (m, 1H, H-5′b); 2.60, 2.40 (2t, 2H, J=7.50 Hz, CH₂CH₂CN); 2.27-2.10 (m, 2H, H-2′); 1.28-1.21 (m, 2H, C—CH₃); 1.19-1.13 (m, 8H, C—CH₃); 1.07-1.03 (m, 2H, C—CH₃).

³¹P NMR (202.5 MHz, CDCl₃ ref. 85% H₃PO₄) δ 148.48, 148.25 in −1:1 ratio.

HRMS (ESI) m/z: [M+H]⁺ Calcd for C₄₇H₅₆N₄O₈P 835.3830. found 835.3836.

### 6.9.2.1. Synthesis of 1-[2-deoxy-β-D-erythro-pentofuranosyl]-1,3,4,7-tetrahydro-2H-1,3-diazepin-2-one (46)

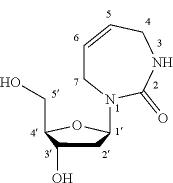

To a stirring solution of compound 45 (4 g, 7.0 mmol) in methanol (400 mL) was added 40 mL of 30% aq. ammonia solution and kept stirring for 3 days at rt. After the disappearance of the starting material, volatiles were removed by rotary vacuum evaporator and residue was co-evaporated again with 100 mL water to remove formed methyl toluate and then freeze dried from water (50 mL) to get the toluoyl deprotected compound (2.25 g). To 500 mg of this product was added 30% aq. ammonia solution (3 mL) and the mixture was kept at rt, for 15 min. After the disappearance of the starting material as evidenced by TLC (40% MeOH in DCM), ammonia was evaporated in vacuo and the crude product was purified by preparative TLC using 20% MeOH in DCM as eluent to afford the required product as a foam (Yield: 0.2 g, 60%).

¹H NMR (500 MHz, D₂O) δ 6.97 (dd, 1H, J=8.51, 6.62 Hz, H-1′), 5.93-5.86 (m, 2H, H-5 and H-6); 4.36-4.32 (m, 1H, H-3′) 3.86-3.75 (m, 4H, H-3 and H-7), 3.75-3.67 (m, 2H, H-5′), 2.20 (ddd, 1H, J=15.10, 8.20, 7.25 Hz, H-2′b); 2.03 (ddd, 1H, J=15.10, 6.60, 3.15 Hz, H-2′a).

### 6.9.3. Evaluation of 7-Membered Ring Nucleosides as CDA Inhibitors Using UV-Vis Based Deamination Assay

The seven-membered ring-containing nucleosides synthesized were tested for their inhibition potential against human CDA enzyme by a UV-Vis based deamination assay. All data were obtained by monitoring absorbance of 2′-deoxycytidine at 286 nm and 25° C., where the deamination of cytosine leads to a decrease in absorption. For nucleosides 34R and 34S, the condition of the assay used was 27 nM human CDA and 100 μM dC as a substrate in 20 mM Na-phosphate buffer, 100 mM NaCl at pH 6.0; an average of 8 measurements was done for each nucleoside. As the original 7-membered ring nucleosides were reported as slow-binding inhibitors of CDA,¹⁰ preincubation conditions of an inhibitor with CDA were also tested for the assay and labelled as ‘preincubation’. Here, the nucleosides were preincubated with the enzyme for 5 min and then 100 μM dC substrate was added to start the measurements (FIG. 12).

These results indicate that compound 34S has some inhibitory potential against human CDA but not 34R which highlights importance of chirality of carbon bearing OH in 7-membered ring. Both compounds have CH₂ instead of N in position 3 of the 7-membered ring, which is probably detrimental for their inhibition of CDA.

The ribose form of compound 46 was reported in the past to be a sub-nM inhibitor of mouse kidney and human liver CDA.¹¹ Here, inventors monitored deamination of 2 mM dC over a period of time after the addition of a known concentration of an inhibitor and the UV absorbance was measured at 286 nm monitoring consumption of dC by 27 nM human CDA. The data was analysed using Lambert's W function as described in Example 5 and Kᵢ values for dZ and compound 46 are provided in the table below.

TABLE 11
Derived Kᵢ values of dZ and 46 determined
by UV-vis based assay against human CDA.

| Inhibitor | Kᵢ (μM) |
|---|---|
| dZ | 10.7 ± 0.5 |
| 46 | 0.43 ± 0.08 |

These experiments showed that 2′-deoxy form of the 7-membered ring nucleoside containing a double bond, namely compound 46 was a more powerful inhibitor of human CDA than dZ.

### 6.9.4. Synthesis of Oligodeoxynucleotides Containing Compounds 34R, 34S and 46 and their Evaluation as APOBEC3 Inhibitors

Oligonucleotides were prepared on a MerMade-4 DNA/RNA synthesizer (BioAutomation) on a 5 μmol scale using standard manufacturer's protocol. Coupling times of modified phosphoramidites 38R, 38S and 45 were increased from 2 to 10 min. The final detritylated oligos were cleaved from the solid support and deprotected at r.t. using conc. NH₄OH. The deprotected oligos in solution were freeze-dried and dry pellets were dissolved in milli-Q water (1 mL) and purified and isolated by reverse-phase HPLC on 250/4.6 mm, 5 μm, 300 Å C18 column (Thermo Fisher Scientific) in a gradient of CH₃CN (0→20% for 20 min, 1.3 mL/min) in 0.1 M TEAA buffer (pH 7.0) with a detection at 260 nm. For the deprotection of the TBDPS protecting group in oligos containing 34R and 34S, the dry pellets were dissolved in a solution of 200 μL Et₃N·3HF. The mixture was kept at 22° C. for 2 hr. The reaction mixture was quenched by the addition of 2 M TEAA buffer (pH 7.0) and oligos were purified by reverse-phase HPLC on 250/4.6 mm, 5 μm, 300 Å C18 column (Thermo Fisher Scientific) in a gradient of CH₃CN (0→80% for 14 min, 1.3 mL/min) in 0.1 M TEAA buffer (pH 7.0) with a detection at 260 nm. Oligonucleotides were freeze-dried, pellets were dissolved in milli-Q water (1.5 mL) and desalted by reverse-phase HPLC on 100/10 mm, 5 μm, 300 Å C18 column (Phenomenex) in a gradient of CH₃CN (0→80% for 15 min, 5 mL/min) in milli-Q water with detection at 260 nm. Pure products were quantified by measuring absorbance at 260 nm, analyzed by ESI-MS (Table 12) and concentrated by freeze-drying.

TABLE 12
List of oligos synthesized.

| | | Retention | ESI-MS |
|---|---|---|---|
| Name | DNA sequence, 5′→3′ | time (min) | [Da] found/calculated |
| 7-mer dZ-linear | TTTTdZAT | 14.2 | 2045.3722/2045.3678 |
| 9-mer 46-linear | ATTT46ATTT | 15.9 | 2679.4839/2679.5024 |
| 7-mer 34R-linear | TTTT34RAT | 13.4 | 2078.3888/2078.4141 |
| 7-mer 34S-linear | TTTT34SAT | 13.4 | 2078.3824/2078.4141 |

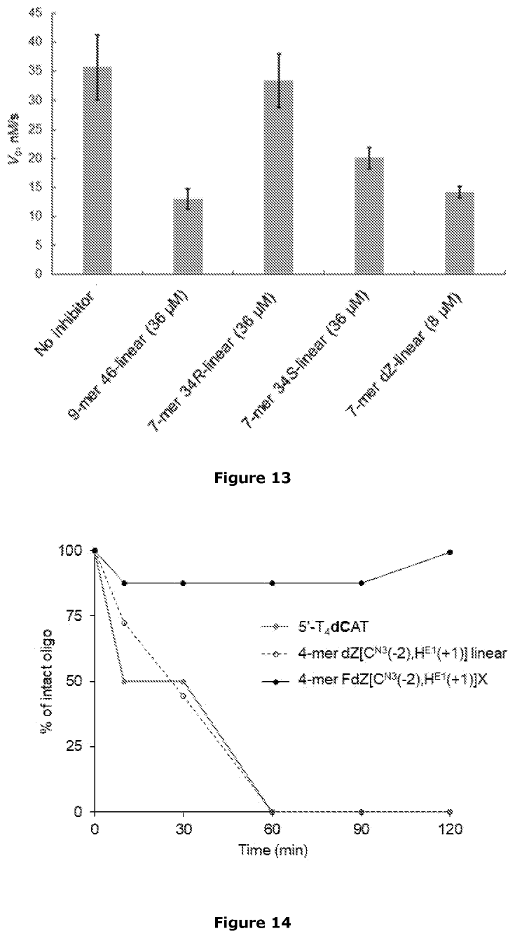

Residual activity of A3B_{CTD}-QM-ΔL3-AL1swap on the unmodified oligo (5′-T₄CAT) as a substrate in the presence of a known concentration of inhibitors was measured using the NMR assay as described in Example 5. Results are shown in FIG. 13.

The results revealed that although nucleoside 46 is as a powerful inhibitor of CDA, when incorporated into a preferred sequence of ssDNA it was not a better inhibitor of A3B_{CTD}-QM-ΔL3-AL1swap compared to the 7-mer dZ-linear. It was observed that 4.5 times the concentration of this oligo gave us a comparable inhibition as that of 7-mer dZ-linear. Interestingly, the inhibition by 7-mer 34R-linear and 7-mer 34S-linear oligos was poor compared to the standard inhibitor (7-mer dZ-linear), but difference in inhibitory potential observed between R and S isomers suggested the position of attack of H₂O relative to the cytosine in A3B_{CTD}-QM-ΔL3-AL1swap. The position of OH relative to the nucleobase was the same as observed in the structure of hydrated FdZ-hairpin with the wild-type APOBEC3A (FIG. 10). The 7-mer 34R-linear and 7-mer 34S-linear oligos were not inhibiting the enzyme efficiently presumably due to the lack of the N3 atom on the nucleobase, which is present in almost all reported transition-state analogues of CDA providing key hydrogen-bonding interactions between nucleobase and the enzyme. These examples provide evidence that once 2-deoxy analogues of nucleoside-like inhibitors of CDA are incorporated into oligos they become inhibitors of APOBEC3 enzymes with variable potency.

### 6.10. Example 9: Evaluation of Inhibitory Potential of Cross-Linked and Hairpin Oligos Against Wild-Type APOBEC3A

To evaluate inhibitory potential of cross-linked and hairpin oligos against wild-type A3, the inventors expressed and purified wild-type APOBEC3A possessing (his)₇ tag at C-terminal end. Inhibitors were tested in the NMR assay monitoring APOBEC3A-catalysed deamination of dC-hairpin-3. Deamination data was analysed using Lambert's W function as described in Example 5. The calculated inhibition constants are provided in Table 13.

TABLE 13
Ki values of inhibitors of wild-type APOBEC3A.

| Inhibitor | Ki (nM) |
|---|---|
| FdZ-linear | 2400 ± 940 |
| FdZ-hairpin-1 | 54 ± 47 |
| 46-hairpin-3 | 290 ± 40 |
| (5'-TGCGCTT-46-GCGCT) | |
| cross-link 2: | 360 ± 120 |
| dZ[U^{E}(2), A^{N3}(+1)]X | |
| cross-link3: | 44 ± 8 |
| FdZ[C^{N3}( ± 2), H^{E1}(+1)]X | |

Conditions: 50 mM Na⁺/K⁺ phosphate buffer, pH 7.4 supplemented with 100 mM NaCl, 1 mM TCEP, 100 μM DSS and 10% D₂O; enzyme concentration: 140 nM; substrate concentration (dC-hairpin-3): 500 μM. Kₘ for dC-hairpin-3 against APOBEC3A is 21 μM, K_{cat} is 0.13 s⁻¹, K_{cat}/Kₘ is 6.2 s⁻¹ mM⁻¹. 46-hairpin-3 has a retention time of 16.7 min (see conditions listed for RP-HPLC in section 6.9.4), ESI-MS: Found: 3917.6466, Calculated: 3917.6906.

Obtained Kᵢ values show that dZ/FdZ-containing hairpin and cross-linked oligos are superior inhibitors of the wild-type APOBEC3A in comparison with the linear oligo (FdZ-linear), which is consistent with the results obtained for A3B_{CTD}-QM-ΔL3-AL1swap in Example 5. It is also evident from this data that compound 46 is a more potent inhibitor of A3 when it is used instead of dC in the structure of hairpin-3 than in the linear 9-mer sequence (FIG. 13). It was also surprising that 46-hairpin-3 had a Kᵢ value in nM range close to that of dZ in cross-link 2. This supports inventors' assertion that nucleoside-like inhibitors of CDA can be converted into inhibitors of A3 when used instead of dC in hairpin and cross-linked oligos.

### 6.11. Example 10: Evaluation of Nuclease Stability of Cross-Linked Oligo

Nuclease stability of modified oligos was evaluated using snake venom phosphodiesterase (phosphodiesterase I, Sigma) and compared with degradation of unmodified sequence 5′-T₄dCAT. Under the conditions used in this experiment, 5′-T₄dCAT and linear 4-mer dZ oligo were completely degraded within 60 min (FIG. 14) whereas cross-linked 4-mer inhibitor FdZ[C^{N3}(−2),H^{E1}(+1)]X showed significantly enhanced stability against enzymatic digestion. These data demonstrate an additional benefit of introduction of a cross-link into DNA-based inhibitors of A3B_{CTD}, meaning that cross-linked oligos will have extended life-time in biological media in comparison with linear oligos.

### 6.12. Discussion

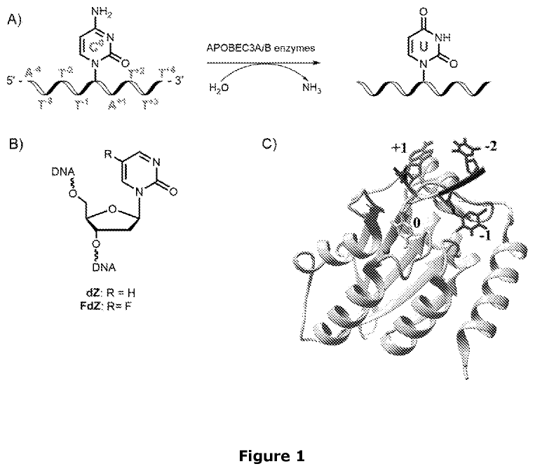

In previous work the inventors developed the first selective A3 inhibitors^{1-2, 45} based on incorporation of cytosine-like 2′-deoxyzebularine^{14, 55} (dZ on FIG. 1B) into short, linear ssDNA, achieving low micromolar inhibition constants (Kᵢ). In their current work the inventors have surprisingly demonstrated that by using DNA hairpin structures or cross-linking dA⁺¹ and T⁻², mimicking the close proximity observed in the crystal structure of A3B_{CTD},⁴⁷ a faster deaminated substrate and a better inhibitor can be obtained in comparison with linear ssDNA of the same length and nucleotide content. These results obtained in the NMR-based activity assay have been corroborated in ITC binding experiments.

The overall inhibition effect was improved from 20-fold (dZ-linear) and 35-fold (FdZ-linear) to more than 400-fold for FdZ-hairpin-1 and 1500-fold for FdZ[C^{N3}(−2),H^{E1}(+1)]X when the apparent inhibition constants (Kᵢ) of oligos are compared with the Kₘ of the linear ssDNA substrate 5′-AT₃dCAT₃ (Kₘ=150 μM, non-linear regression analysis) for A3B_{CTD}-QM-ΔL3-AL1swap. When the best inhibitor, FdZ[C^{N3}(−2),H^{E1}(+1)]X, was evaluated against preferred substrate of A3B_{CTD}, dC-hairpin, a similar inhibition effect of ˜1000-fold was observed, i.e. Kₘ (dC-hairpin)/Kᵢ (FdZ[C^{N3}(−2),H^{E1}(+1)]X), Table 6. A similar pattern of inhibition by hairpin and cross-linked oligos was observed against wild-type APOBEC3A enzyme (Table 13), which suggests that these inhibitors can be used against wild-type APOBEC3A and APOBEC3B. As Kₘ and Kᵢ depend strongly on the surrounding environment (for example, buffer composition, pH and ionic strength) inhibition with even lower Kᵢ may be observed in cellular environments, because Kₘ/K_{d} values in low μM¹⁸⁻¹⁹ and nM⁵⁶ range have been reported.

Inventors observed in crystal structure that FdZ being a part of an oligo is hydrated by wild-type APOBEC3A in a similar way as it was observed in the past in the crystal structure of 5-fluorozebularine bound to CDA. This observation along with correlation of inhibitory data of compounds 34R, 34S and 46 against human CDA and of compounds 9-mer 46-linear, 7-mer 34R-linear and 7-mer 34S-linear against A3B_{CTD}-QM-ΔL3-AL1swap support inventors' argument that 2-deoxy forms of nucleoside-like inhibitors of CDA can be converted to APOBEC3 inhibitors after their incorporation into the preferred DNA substrate instead of the target dC.

Additionally, without wishing to be bound by theory, the inventors believe that the cross-linked oligos described herein, by virtue of their unique shape, will not interact strongly with other proteins and will not be able to form duplexes or other secondary structures with partially or fully complementary genomic sequences. In this manner, the therapeutic use of the cross-linked oligos described herein is supported by a potential reduction in toxicity, making these inhibitors suitable for clinical applications.

Moreover, the cross-linked 4-mer oligos described herein show enhanced stability towards enzymatic digestion by phosphodiesterase I, which indicates that these inhibitors will have extended life-time in biological media.

### 7. INDUSTRIAL APPLICABILITY

Inhibition of APOBEC3 enzymes, especially A3A and A3B, is clinically relevant as these enzymes are involved in cancer mutagenesis and development of drug resistance. In this study the inventors developed ssDNAs that are better substrates for A3 enzymes using a cross-linking strategy employing Cu(I)-catalyzed ‘click chemistry’ between alkyne and azide moieties embedded in a ssDNA fragment. Based on the work disclosed herein, the inventors have unexpectedly made the first nM inhibitors of A3B_{CTD} and wild-type A3A by incorporating C-to-U inhibitor dZ or FdZ in place of the target dC in the cross-linked DNA fragments and DNA hairpins.

### 8. REFERENCES

1. Kvach, M. V.; Barzak, F. M.; Harjes, S.; Schares, H. A. M.; Jameson, G. B.; Ayoub, A. M.; Moorthy, R.; Aihara, H.; Harris, R. S.; Filichev, V. V.; Harki, D. A.; Harjes, E., Inhibiting APOBEC3 Activity with Single-Stranded DNA Containing 2′-Deoxyzebularine Analogues. Biochemistry 2019, 58 (5), 391-400.
2. Kvach, M. V.; Barzak, F. M.; Harjes, S.; Schares, H. A. M.; Kurup, H. M.; Jones, K. F.; Sutton, L.; Donahue, J.; D'Aquila, R. T.; Jameson, G. B.; Harki, D. A.; Krause, K. L.; Harjes, E.; Filichev, V. V., Differential Inhibition of APOBEC3 DNA-Mutator Isozymes by Fluoro- and Non-Fluoro-Substituted 2′-Deoxyzebularine Embedded in Single-Stranded DNA. ChemBioChem 2020, 21 (7), 1028-1035.
3. Law, E. K.; Sieuwerts, A. M.; LaPara, K.; Leonard, B.; Starrett, G. J.; Molan, A. M.; Temiz, N. A.; Vogel, R. I.; Meijer-van Gelder, M. E.; Sweep, F. C. G. J.; Span, P. N.; Foekens, J. A.; Martens, J. W. M.; Yee, D.; Harris, R. S., The DNA cytosine deaminase APOBEC3B promotes tamoxifen resistance in ER-positive breast cancer. Sci. Adv. 2016, 2 (10), e1601737.
4. Astakhova, I. K.; Santhosh Kumar, T.; Campbell, M. A.; Ustinov, A. V.; Korshun, V. A.; Wengel, J., Branched DNA nanostructures efficiently stabilised and monitored by novel pyrene-perylene 2′-α-L-amino-LNA FRET pairs. ChemComm 2013, 49 (5), 511-513.
5. Graham, D.; Parkinson, J. A.; Brown, T., DNA duplexes stabilized by modified monomer residues: synthesis and stability. J. Chem. Soc. Perkin Trans. 1 1998, (6), 1131-1138.
6. Betts, L.; Xiang, S.; Short, S. A.; Wolfenden, R.; Carter, C. W., Jr., Cytidine deaminase. The 2.3 Å crystal structure of an enzyme: transition-state analog complex. J. Mol. Biol. 1994, 235 (2), 635-56.
7. Frick, L.; Yang, C.; Marquez, V. E.; Wolfenden, R. V., Binding of pyrimidin-2-one ribonucleoside by cytidine deaminase as the transition-state analog 3,4-dihydrouridine and contribution of the 4-hydroxyl group to its binding affinity. Biochemistry 1989, 28 (24), 9423-9430.
8. Borchers, C. H.; Marquez, V. E.; Schroeder, G. K.; Short, S. A.; Snider, M. J.; Speir, J. P.; Wolfenden, R., Fourier transform ion cyclotron resonance MS reveals the presence of a water molecule in an enzyme transition-state analogue complex. Proc. Natl. Acad. Sci. U.S.A. 2004, 101 (43), 15341-15345.
9. Schroeder, G. K.; Zhou, L.; Snider, M. J.; Chen, X.; Wolfenden, R., Flight of a Cytidine Deaminase Complex with an Imperfect Transition State Analogue Inhibitor: Mass Spectrometric Evidence for the Presence of a Trapped Water Molecule. Biochemistry 2012, 51 (32), 6476-6486.
10. Marquez, V. E.; Liu, P. S.; Kelley, J. A.; Driscoll, J. S.; Mccormack, J. J., Synthesis of 1,3-diazepin-2-one nucleosides as transition-state inhibitors of cytidine deaminase. J. Med. Chem. 1980, 23 (7), 713-715.
11. Liu, P. S.; Marquez, V. E.; Driscoll, J. S.; Fuller, R. W.; Mccormack, J. J., Cyclic urea nucleosides. Cytidine deaminase activity as a function of aglycon ring size. J. Med. Chem. 1981, 24 (6), 662-666.
12. Marquez, V. E.; Rao, K. V. B.; Silverton, J. V.; Kelley, J. A., A ring expansion approach to 1,3-diazepin-2-one nucleosides. J. Org. Chem. 1984, 49 (5), 912-919.
13. Kim, C. H.; Marquez, V. E.; Mao, D. T.; Haines, D. R.; Mccormack, J. J., Synthesis of pyrimidin-2-one nucleosides as acid-stable inhibitors of cytidine deaminase. J. Med. Chem. 1986, 29 (8), 1374-80.
14. Barchi, J. J.; Haces, A.; Marquez, V. E.; Mccormack, J. J., Inhibition of Cytidine Deaminase by Derivatives of 1-(β-D-Ribofuranosyl)-Dihydropyrimidin-2-One (Zebularine). Nucleosides Nucleotides 1992, 11 (10), 1781-1793.
15. Jeong, L. S.; Buenger, G.; Mccormack, J. J.; Cooney, D. A.; Hao, Z.; Marquez, V. E., Carbocyclic Analogues of the Potent Cytidine Deaminase Inhibitor 1-(β-d-Ribofuranosyl)-1,2-dihydropyrimidin-2-one (Zebularine). J. Med. Chem. 1998, 41 (14), 2572-2578.
16. Kim, M.; Gajulapati, K.; Kim, C.; Jung, H. Y.; Goo, J.; Lee, K.; Kaur, N.; Kang, H. J.; Chung, S. J.; Choi, Y., A facile synthetic route to diazepinone derivatives via ring closing metathesis and its application for human cytidine deaminase inhibitors. ChemComm 2012, 48 (93), 11443-11445.
17. Buisson, R.; Langenbucher, A.; Bowen, D.; Kwan, E. E.; Benes, C. H.; Zou, L.; Lawrence, M. S., Passenger hotspot mutations in cancer driven by APOBEC3A and mesoscale genomic features. Science 2019, 364 (6447), eaaw2872.
18. Silvas, T. V.; Hou, S.; Myint, W.; Nalivaika, E.; Somasundaran, M.; Kelch, B. A.; Matsuo, H.; Kurt Yilmaz, N.; Schiffer, C. A., Substrate sequence selectivity of APOBEC3A implicates intra-DNA interactions. Sci. Rep. 2018, 8 (1), 7511.
19. Hou, S.; Silvas, T. V.; Leidner, F.; Nalivaika, E. A.; Matsuo, H.; Kurt Yilmaz, N.; Schiffer, C. A., Structural Analysis of the Active Site and DNA Binding of Human Cytidine Deaminase APOBEC3B. J. Chem. Theory Comput. 2019, 15 (1), 637-647.
20. Yang, H.; Tang, J. A.; Greenberg, M. M., Synthesis of Oligonucleotides Containing the N(6)-(2-Deoxy-a,b-D-erythropentofuranosyl)-2,6-diamino-4-hydroxy-5-formamidopyrimidine (FapydG) Oxidative Damage Product Derived from 2′-Deoxyguanosine. Chem.-Eur. J. 2020, 26 (24), 5441-5448.
21. Štimac, A.; Kobe, J., Stereoselective synthesis of 1,2-cis- and 2-deoxyglycofuranosyl azides from glycosyl halides. Carbohydr. Res. 2000, 329 (2), 317-324.
22. Damha, M. J.; Giannaris, P. A.; Zabarylo, S. V., An improved procedure for derivatization of controlled-pore glass beads for solid-phase oligonucleotide synthesis. Nucleic Acids Res. 1990, 18 (13), 3813-3821.
23. Pourceau, G.; Meyer, A.; Vasseur, J. J.; Morvan, F., Azide Solid Support for 3′-Conjugation of Oligonucleotides and Their Circularization by Click Chemistry. J. Org. Chem. 2009, 74 (17), 6837-6842.
24. Vives, M.; Eritja, R.; Tauler, R.; Marquez, V.; Gargallo, R., Synthesis, stability, and protonation studies of a self-complementary dodecamer containing the modified nucleoside 2′-deoxyzebularine. Biopolymers: Original Research on Biomolecules 2004, 73 (1), 27-43.
25. Ottria, R.; Casati, S.; Baldoli, E.; Maier, J. A. M.; Ciuffreda, P., N6-Alkyladenosines: Synthesis and evaluation of in vitro anticancer activity. Bioorg. Med. Chem. 2010, 18 (23), 8396-8402.
26. Tararov, V. I.; Kolyachkina, S. V.; Alexeev, C. S.; Mikhailov, S. N., N6-Acetyl-2′,3′,5′-tri-O-acetyladenosine; A Convenient, ‘Missed Out’ Substrate for Regioselective N6-Alkylations. Synthesis 2011, 2011 (15), 2483-2489.
27. Sugai, N.; Heguri, H.; Ohta, K.; Meng, Q.; Yamamoto, T.; Tezuka, Y., Effective Click Construction of Bridged- and Spiro-Multicyclic Polymer Topologies with Tailored Cyclic Prepolymers (kyklo-Telechelics). J. Am. Chem. Soc. 2010, 132 (42), 14790-14802.
28. Fomich, M. A.; Kvach, M. V.; Navakouski, M. J.; Weise, C.; Baranovsky, A. V.; Korshun, V. A.; Shmanai, V. V., Azide Phosphoramidite in Direct Synthesis of Azide-Modified Oligonucleotides. Org. Lett. 2014, 16 (17), 4590-4593.
29. Fall, A.; Seck, I.; Diouf, O.; Gaye, M.; Seck, M.; Gomez, G.; Fall, Y., Synthesis of new triazolium-based ionic liquids and their use in the Morita-Baylis-Hillman reaction. Tetrahedron letters 2015, 56 (36), 5128-5131.
30. Miah, A.; Reese, C. B.; Song, Q., Convenient Intermediates for the Preparation of C-4 Modified Derivatives of Pyrimidine Nucleosides. Nucleosides Nucleotides 1997, 16 (1-2), 53-65.
31. Budhathoki-Uprety, J.; Jena, P. V.; Roxbury, D.; Heller, D. A., Helical polycarbodiimide cloaking of carbon nanotubes enables inter-nanotube exciton energy transfer modulation. J. Am. Chem. Soc. 2014, 136 (44), 15545-15550.
32. Stawinski, J.; Stromberg, R., Deoxyribo- and Ribonucleoside H-Phosphonates. Curr. Protoc. Nucleic Acid Chem. 2001, 4 (1), 2.6.1-2.6.15.
33. Bonesi, S. M.; Erra-Balsells, R., Synthesis and isolation of iodocarbazoles. Direct iodination of carbazoles by N-iodosuccinimide and N-iodosuccinimide-silica gel system. Journal of Heterocyclic Chemistry 2001, 38 (1), 77-87.
34. Hong, I. S.; Ding, H.; Greenberg, M. M., Oxygen Independent DNA Interstrand Cross-Link Formation by a Nucleotide Radical. J. Am. Chem. Soc. 2006, 128 (2), 485-491.
35. Shibata, A.; Ueno, Y.; Iwata, M.; Wakita, H.; Matsuda, A.; Kitade, Y., Double-stranded oligonucleotides containing 5-aminomethyl-2′-deoxyuridine form thermostable anti-parallel triplexes with single-stranded DNA or RNA. Bioorg. Med. Chem. Lett. 2012, 22 (8), 2681-2683.
36. Iyidogan, P.; Sullivan, T. J.; Chordia, M. D.; Frey, K. M.; Anderson, K. S., Design, Synthesis, and Antiviral Evaluation of Chimeric Inhibitors of HIV Reverse Transcriptase. ACS Medicinal Chemistry Letters 2013, 4 (12), 1183-1188.
37. Ries, A.; Kumar, R.; Lou, C.; Kosbar, T.; Vengut-Climent, E.; Jorgensen, P. T.; Morales, J. C.; Wengel, J., Synthesis and Biophysical Investigations of Oligonucleotides Containing Galactose-Modified DNA, LNA, and 2′-Amino-LNA Monomers. J. Org. Chem. 2016, 81 (22), 10845-10856.
38. Ingale, S. A.; Leonard, P.; Yang, H.; Seela, F., 5-Nitroindole oligonucleotides with alkynyl side chains: universal base pairing, triple bond hydration and properties of pyrene “click” adducts. Org. Biomol. Chem. 2014, 12 (42), 8519-8532.
39. Ingale, S. A.; Mei, H.; Leonard, P.; Seela, F., Ethynyl side chain hydration during synthesis and workup of “clickable” oligonucleotides: bypassing acetyl group formation by triisopropylsilyl protection. J. Org. Chem. 2013, 78 (22), 11271-11282.
40. Barchi, J. J., Jr.; Musser, S.; Marquez, V. E., The decomposition of 1-(β-D-ribofuranosyl)-1,2-dihydropyrimidin-2-one (zebularine) in alkali: mechanism and products. J. Org. Chem. 1992, 57 (2), 536-41.
41. Myers, A. G.; Goldberg, S. D., Synthesis of the Kedarcidin Core Structure by a Transannular Cyclization Pathway. Angew. Chem. Int. Ed. Engl. 2000, 39 (15), 2732-2735.
42. Filichev, V. V.; Pedersen, E. B., Stable and selective formation of Hoogsteen-type triplexes and duplexes using twisted intercalating nucleic acids (TINA) prepared via postsynthetic Sonogashira solid-phase coupling reactions. J. Am. Chem. Soc. 2005, 127 (42), 14849-14858.
43. Rist, M.; Amann, N.; Wagenknecht, H. A., Preparation of 1-ethynylpyrene-modified DNA via Sonogashira-type solid-phase couplings and characterization of the fluorescence properties for electron-transfer studies. Eur. J. Org. Chem. 2003, (13), 2498-2504.
44. Filichev, V. V.; Astakhova, I. V.; Malakhov, A. D.; Korshun, V. A.; Pedersen, E. B., 1-, 2-, and 4-Ethynylpyrenes in the Structure of Twisted Intercalating Nucleic Acids: Structure, Thermal Stability, and Fluorescence Relationship. Chem.-Eur. J. 2008, 14 (32), 9968-9980.
45. Barzak, F. M.; Harjes, S.; Kvach, M. V.; Kurup, H. M.; Jameson, G. B.; Filichev, V. V.; Harjes, E., Selective inhibition of APOBEC3 enzymes by single-stranded DNAs containing 2′-deoxyzebularine. Org. Biomol. Chem. 2019, 17 (43), 9435-9441.
46. Paar, M.; Schrabmair, W.; Mairold, M.; Oettl, K.; Reibnegger, G., Global Regression Using the Explicit Solution of Michaelis-Menten Kinetics Employing Lambert's W Function: High Robustness of Parameter Estimates. ChemistrySelect 2019, 4 (6), 1903-1908.
47. Shi, K.; Carpenter, M. A.; Banerjee, S.; Shaban, N. M.; Kurahashi, K.; Salamango, D. J.; Mccann, J. L.; Starrett, G. J.; Duffy, J. V.; Demir, O.; Amaro, R. E.; Harki, D. A.; Harris, R. S.; Aihara, H., Structural basis for targeted DNA cytosine deamination and mutagenesis by APOBEC3A and APOBEC3B. Nat. Struct. Mol. Biol. 2017, 24 (2), 131-139.
48. Iocono, J. A.; Gildea, B.; McLaughlin, L. W., Mild acid hydrolysis of 2-pyrimidinone-containing DNA fragments generates apurinic/apyrimidinic sites. Tetrahedron Lett. 1990, 31 (2), 175-8.
49. Connolly, B. A.; Newman, P. C., Synthesis and properties of oligonucleotides containing 4-thiothyniidine, 5-methyl-2-pyrunidinone-1-β-D(2′-dexoyriboside) and 2-thiothvmidine. Nucleic Acids Res. 1989, 17 (13), 4957-4974.
50. Gildea, B.; McLaughlin, L. W., The synthesis of 2-pyrimidinone nucleosides and their incorporation into oligodeoxynucleotides. Nucleic Acids Res. 1989, 17 (6), 2261-81.
51. Rappaport, H. P., The 6-thioguanine/5-methyl-2-pyrimidinone base pair. Nucleic Acids Res. 1988, 16 (15), 7253-7267.
52. Tarkin-Tas, E.; Mathias, L. J., Synthesis and Ring-Opening Polymerization of 5-Azepane-2-one Ethylene Ketal: A New Route to Functional Aliphatic Polyamides. Macromolecules 2010, 43 (2), 968-974.
53. Kotick, M. P.; Szantay, C.; Bardos, T. J., Synthesis of 5-s-substituted 2′-deoxyuridines. Study of the factors influencing the stereoselectivity of the silyl modification of the Hilbert-Johnson reaction. J. Org. Chem. 1969, 34 (12), 3806-3813.
54. Ludek, O. R.; Schroeder, G. K.; Liao, C.; Russ, P. L.; Wolfenden, R.; Marquez, V. E., Synthesis and Conformational Analysis of Locked Carbocyclic Analogues of 1,3-Diazepinone Riboside, a High-Affinity Cytidine Deaminase Inhibitor. J. Org. Chem. 2009, 74 (16), 6212-6223.
55. Marquez, V. E.; Barchi, J. J., Jr.; Kelley, J. A.; Rao, K. V.; Agbaria, R.; Ben-Kasus, T.; Cheng, J. C.; Yoo, C. B.; Jones, P. A., Zebularine: a unique molecule for an epigenetically based strategy in cancer chemotherapy. The magic of its chemistry and biology. Nucleosides Nucleotides Nucl. Acids 2005, 24 (5-7), 305-318.
56. Fu, Y.; Ito, F.; Zhang, G.; Fernandez, B.; Yang, H.; Chen, X. S., DNA cytosine and methylcytosine deamination by APOBEC3B: enhancing methylcytosine deamination by engineering APOBEC3B. Biochem. J. 2015, 471 (1), 25-35.
57. Fieser and Fieser's Reagents for Organic Synthesis, Volumes 1-15 (John Wiley and Sons, 1991).
58. Organic Reactions, Volumes 1-40 (John Wiley and Sons, 1991).
59. March's Advanced Organic Chemistry, (John Wiley and Sons, 4th Edition).
60. Larock's Comprehensive Organic Transformations (VCH Publishers Inc., 1989).
61. T. W. Greene and G. M. Wuts, Protecting Groups in Organic Synthesis, Third Edition, Wiley, New York, 1999)
62. Brown, D. M. A brief history of oligonucleotide synthesis. Methods in Molecular Biology (Totowa, NJ, United States) (1993), 20 (Protocols for Oligonucleotides and Analogs), 1-17.
63. Nucleic acids in chemistry and biology, Ed.: G. M. Blackburn, M. J. Gait, D. Loakes, D. W. Williams, 3^{rd} edition, RSC, 2006.
64. Fantoni, N Z, El-Sagheer, A H, Brown, T. (2021) A Hitchhiker's Guide to Click-Chemistry with Nucleic Acids, Chem. Rev., doi: 10.1021/acs.chemrev.0c00928
65. El-Sagheer, A H, Brown, T. (2010) Click chemistry with DNA, Chem. Soc. Rev. 39, 1388-405

1. A compound of formula I 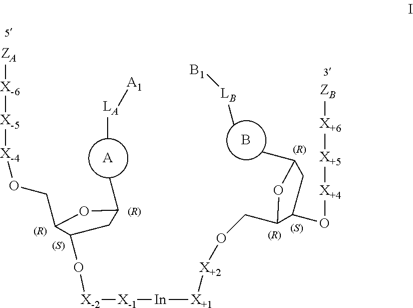
wherein
A₁ and B₁ are selected from —N₃ and —C≡CH wherein A₁ and B₁ are different; L_{A} and L_{B} are independently absent or are independently selected from the group consisting of —(C₁-C₆)alkylene, —(C₁-C₆)alkenylene and —(C₁-C₆)alkynylene, wherein each of —(C₁-C₆)alkylene, —(C₁-C₆)alkenylene and —(C₁-C₆)alkynylene may incorporate one or two heteroatoms and/or an aryl group in the carbon chain;
the A and B rings are independently selected from a substituted or non-substituted 5-14 membered N-containing monocyclic or polycyclic heteroaryl group, wherein each A and B ring is attached to the β-D-2′-deoxyribofuranosyl unit via a N atom present in each of the A and B rings;
each of X₋₆, X₋₅, X₋₄, X₋₂, X₋₁, X₊₁, X₊₂, X₊₄, X₊₅ and X₊₆ is absent or is independently selected from a nucleotide or a nucleotide derivative;
Z_{A} and Z_{B} are each independently absent or selected from an oligonucleotide consisting of 1 to 10 nucleotides and/or nucleotide derivatives; and
In is a D-2-deoxyribose analogue of a nucleoside-like inhibitor of cytidine deaminase.

2. A compound of claim 1 wherein In is selected from the group consisting of 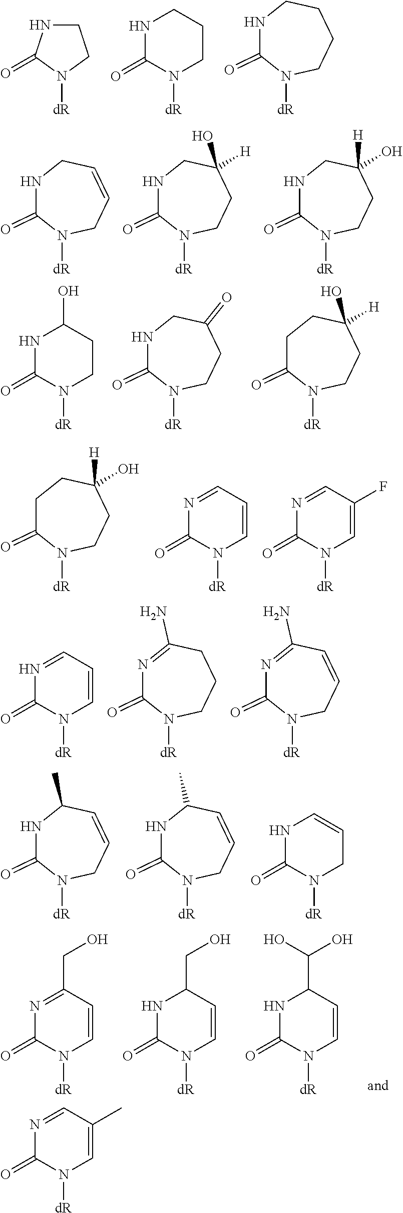
wherein dR is β-D-5-O-phosphato-2-deoxyribofuranosyl as depicted in formula I′ wherein * denotes the point of attachment to X₋₂ or X₋₁, ** denotes the point of attachment to X₊₁ or X₊₂, and *** denotes the point of attachment to the N atom of the In group

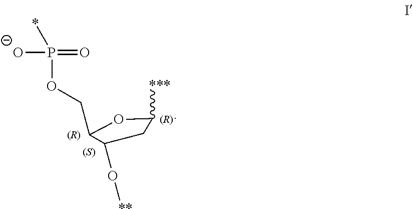

3. A compound of formula IIa or IIb 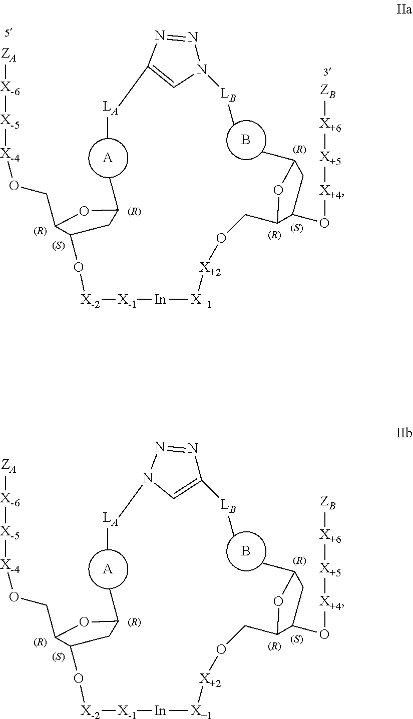
wherein L_{A}, L_{B}, A, B, X₋₆, X₋₅, X₋₄, X₋₂, X₋₁, X₊₁, X₊₂, X₊₄, X₊₅, X₊₆ and In are as defined in claim 1.

4. A compound of claim 3 in which one or both of L_{A} and L_{B} are absent or selected from the group consisting of —(C₁-C₆)alkylene, meta-ethynylbenzen-1-yl and 5-hexynyl-1-yl.

5. A compound of claim 3 in which each of X₋₆, X₋₅, X₋₄, X₋₂, X₋₁, X₊₁, X₊₂, X₊₄, X₊₅ and X₊₆ is a native nucleotide.

6. A compound of claim 3 in which the A and B rings are independently selected from the group consisting of (a) substituted purinyl, (b) substituted 7-deazapurinyl, (c) substituted 8-aza-7-deazapurinyl, (d) substituted pyrimidinyl and (e) substituted tricyclic or dicyclic nitrogen-containing heteroaryl, wherein each of the A and B rings is independently substituted with one or more substituents selected from the group consisting of halo, nitro, cyano, alkyl, alkoxy, amino, substituted amino, —COH, —COOH, and —CONH₂.

7. A compound of claim 3 in which the A and B rings are independently selected from the group consisting of (a) a modified purinyl selected from the group consisting of: 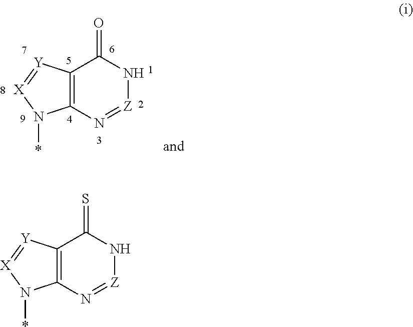
wherein X═C—R¹,Y═N and 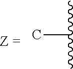
or X═N, Y═C—R¹ and 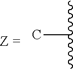
or X═C—R¹, 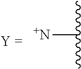 [SMILES: *[NH3+]]
and Z═C—R² or X═N, 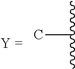
and Z═C—R¹ or 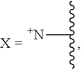 [SMILES: *[NH3+]]
Y═C—R¹ and Z═C—R² or 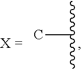
Y═N and Z═C—R¹; 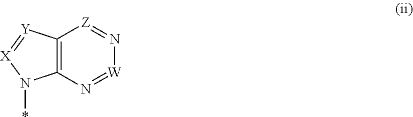
wherein X═C—R¹, Y═N, 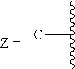
and W═C—R² or X═N, Y═C—R¹, 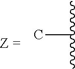
and W═C—R² or X═C—R¹, 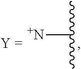 [SMILES: *[NH3+]]
Z═C—R² and W═C—R³ or X═N, 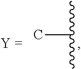
Z═C—R¹ and W═C—R² or 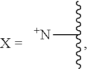 [SMILES: *[NH3+]]
Y═C—R¹, Z═C—R² and W═C—R³ or 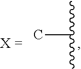
Y═N Z═C—R¹ and W═C—R² or X═C—R¹, Y═N, Z═C—R² and 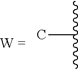
or X═N, Y═C—R¹, Z═C—R² and 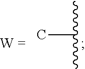
X═C—R¹, Y═N, 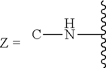 [SMILES: *NC]
and W═C—R² or X═C—R, Y═N, Z═C—R² and 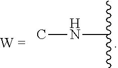 [SMILES: *NC] 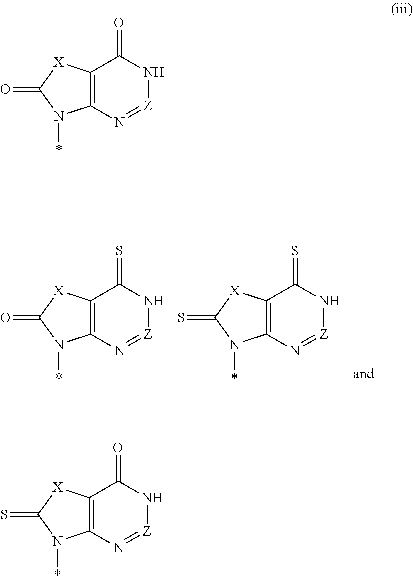
wherein X═C—R¹ and 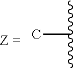
or X═N and 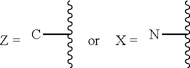
and Z═C—R¹ or 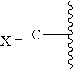
and Z═C—R¹; 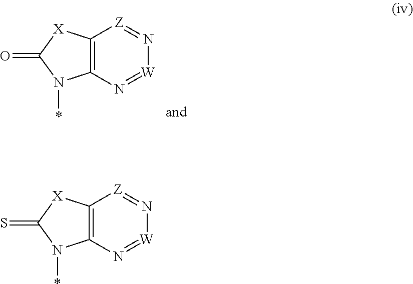
wherein X═C—R¹, 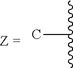
and W═C—R² or X═N, 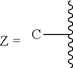
and W═C—R¹ or 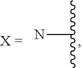 [SMILES: *N]
Z═C—R¹ and W═C—R² or 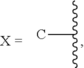
Z═C—R¹ and W═C—R² or X═C—R¹, Z═C—R² and 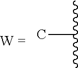
or X═N, Z═C—R¹ and 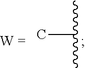 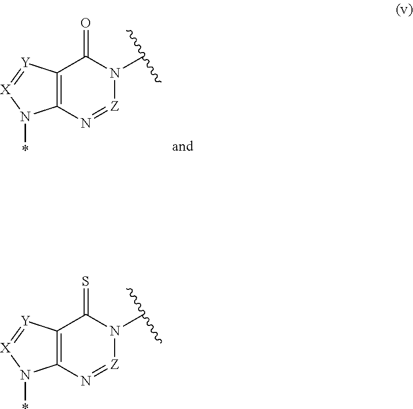
wherein X═N, Y═C—R¹ and Z═C—R² or X═C—R¹, Y═N and Z═C—R²; 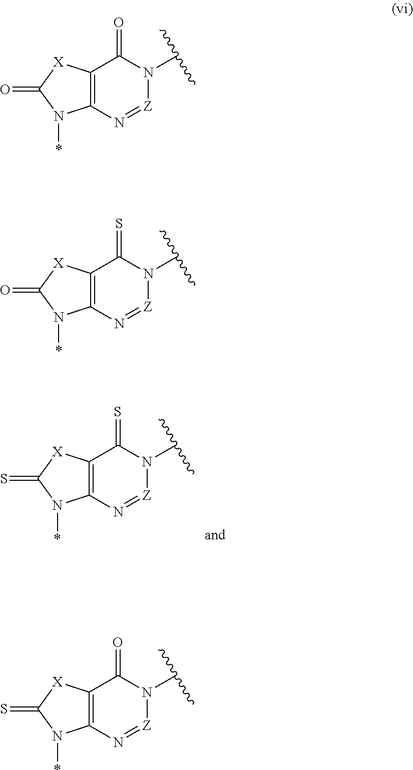
wherein X═C—R¹ and Z═C—R² or X═N and Z═C—R¹;
and wherein R¹, R² and R³ are independently selected from the group consisting of H, halo, nitro, cyano, alkyl, alkoxy, amino, mono or disubstituted amino, —COH, —COOH, and —CONH₂.
(b) a modified pyrimidine selected from the group consisting of: 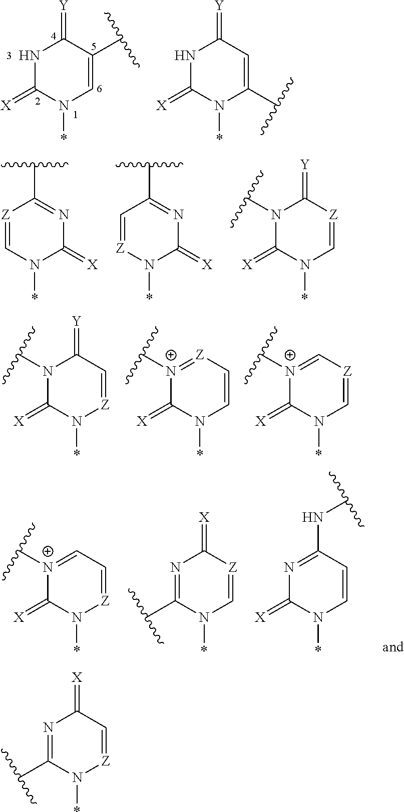
wherein X and Y are independently selected from O or S and Z is independently selected from N or C—R¹ wherein R¹ is independently selected from the group consisting of H, halo, nitro, cyano, alkyl, alkoxy, amino, mono or disubstituted amino, —COH, —COOH, and —CONH₂;
(c) a substituted tricyclic or dicyclic or monocyclic nitrogen-containing heteroaryl selected from the group consisting of: 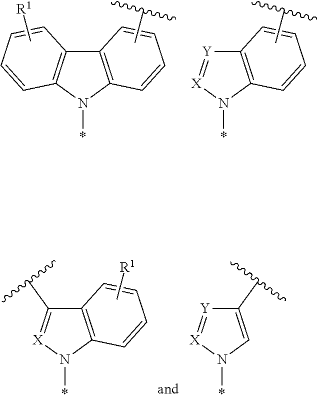
wherein X and Y are independently selected from N, O, S or C—R¹ wherein R¹ is independently selected from the group consisting of H, halo, nitro, cyano, alkyl, alkoxy, amino, mono or disubstituted amino, —COH, —COOH, and —CONH₂;
wherein for each of (a), (b) and (c) 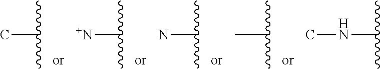
indicates the point of attachment of the ring to L_{A} or L_{B} and * indicates the point of attachment to the β-D-2′-deoxyribofuranosyl unit I′ of formula I or II.

8. A compound of claim 7 in which the A and B rings are independently selected from the group consisting of 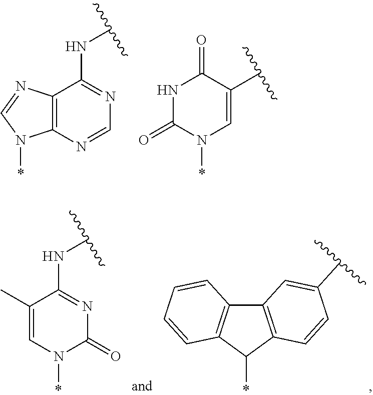
wherein [symbol] indicates the point of attachment of the ring to L_{A} or L_{B} and * indicates the point of attachment to the β-D-2′-deoxyribofuranosyl unit of formula I or II.

9. A compound of claim 3 in which In is 2′-deoxyzebularine (dZ) or 5-fluoro-2′-deoxyzebularine (FdZ).

10. A compound of claim 3 in which one or more of X₋₆, X₋₅, X₋₄, X₋₂, X₋₁, X₊₆, X₊₅, X₊₄, X₊₂ and X₊₁ are absent.

11. A compound of claim 3 in which Z_{A} and/or Z_{B} are absent.

12. A compound of formula IX
5′ Z_{A}—Xₖ—Wₗ—In—Yₙ—Z_{B} 3′ IX
wherein
W is a nucleotide or derivative thereof, or is absent and 1 is selected from 1-5;
Xₖ is a chain of nucleotides or derivatives thereof which are complementary or partially complementary to a chain of nucleotides or derivatives thereof Yₙ, wherein each of k and n is independently selected from 3-10; and
Z_{A}, Z_{B} and In are as defined in claim 1; and
the compound of formula IX forms a unimolecular hairpin in solution in which W—In forms a loop and Xₖ and Yn form a stem.

13. A compound of claim 12 in which In is selected from the group consisting of 2′-deoxyzebularine (dZ), 5-fluoro-2′-deoxyzebularine (FdZ) and 1-[2-deoxy-β-D-erythro-pentofuranosyl]-1,3,4,7-tetrahydro-2H-1,3-diazepin-2-one.

14. A compound of claim 12 in which k=n and is an integer between 3 and 5 and 1 is an integer between 1 and 3.

15. A compound of claim 12 wherein the compound of formula IX is a compound of formulae X-XIII
5′ G-C-G-C-T-T-In-G-C-G-C 3′ X
5′ A-A-G-C-T-T-T-In-A-G-C-T-T 3′ XI
5′ T-G-C-G-C-T-T-In-G-C-G-C-T 3′ XII
5′ C-C-C-A-T-C-A-T-T-In-G-A-T-G-G-G 3′ XIII
wherein In is as defined in claim 1.

16. (canceled)

17. (canceled)

18. (canceled)

19. (canceled)
